(19) United States
(12) Patent Application Publication
(10) Pub. No.: US 2023/0389854 A1
(43) Pub. Date: Dec. 7, 2023

(54) NEUROMELANIN-SENSITIVE MRI AND METHODS OF USE THEREOF

(71) Applicants: Terran Biosciences Inc., New York, NY (US); UNIVERSITY OF OTTAWA INSTITUTE OF MENTAL HEALTH RESEARCH, Ottawa (CA); THE ROYAL INSTITUTION FOR THE ADVANCEMENT OF LEARNING/MCGILL UNIVERSITY, Montreal (CA); The Trustees of Columbia University in the City of New York, New York, NY (US); The Research Foundation for Mental Hygiene, Inc., Menands, NY (US)

(72) Inventors: Samuel CLARK, New York, NY (US); Clifford Mills CASSIDY, Ottawa (CA); Pedro ROSA-NETO, Montreal (CA); Kenneth WENGLER, New York, NY (US); Guillermo HORGA HERNANDEZ, New York, NY (US)

(21) Appl. No.: 18/317,824

(22) Filed: May 15, 2023

Related U.S. Application Data
(63) Continuation of application No. PCT/US2021/059590, filed on Nov. 16, 2021.
(60) Provisional application No. 63/277,490, filed on Nov. 9, 2021.
(60) Provisional application No. 63/120,105, filed on Dec. 1, 2020.
(60) Provisional application No. 63/114,304, filed on Nov. 16, 2020.

(51) Int. Cl. A61B 5/00; A61B 5/055

(52) U.S. Cl. CPC A61B 5/4088; A61B 5/4842; A61B 5/0042; A61B 5/055

(57) ABSTRACT

A neuromelanin sensitive magnetic resonance imaging (“MRI”) technique, method and computer-accessible medium for measuring the extent of, providing a diagnosis of, monitoring the treatment of, assessing novel treatments for, or determining a prognosis related to one or more neurological conditions.

### CROSS REFERENCE TO RELATED APPLICATIONS

This application claims priority to, and the benefit of, U.S. Provisional Application Nos. 63/114,304, filed on Nov. 16, 2020, 63/120,105, filed on Dec. 1, 2020, and 63/277,490, filed on Nov. 9, 2021, the contents of each of which are incorporated by reference herein in their entireties.

### FIELD OF THE DISCLOSURE

The present disclosure relates generally to magnetic resonance imaging (“MRI”), and more specifically, to exemplary embodiments of an exemplary system, method and computer-accessible medium for a neuromelanin-sensitive MRI technique as a non-invasive measure of neurological conditions.

### BACKGROUND

Alzheimer's disease (AD) is one of the common forms of neurodegenerative diseases resulting in dementia, also known as senile dementia of the Alzheimer type and primary degenerative dementia of the Alzheimer's type, Alzheimer disease (AD). The dementia is a huge public health concern, with a new case diagnosed somewhere in the world every 7 seconds. It described by German psychiatrist and neuropathologist Alois Alzheimer in 1906 and named after him. There is no cure for the disease, which worsens as it progresses, and eventually leads to death within 7 years. Less than three percent of individuals live more than fourteen years after diagnosis. People diagnosed as having AD are usually over 65 years of age diagnosed by standard verbal and visual memory tests, decision-making and problem-solving tasks. In 2006, there were 26.6 million sufferers worldwide and 5 million of them in the USA. Alzheimer's disease predicted to affect 1 in 85 people globally by 2050. Early symptoms often erroneously thought to be ‘age-related’ concerns, or manifestations of stress. When AD suspected, the diagnosis usually confirmed with tests that evaluate behavior, memory, cognition, and thinking abilities, followed by brain scan studies.

The neurodegenerative diseases divided into two all-encompassing wide categories of brain afflictions. The diseases are imprecisely divided into two groups: 1. Conditions affecting memory that are ordinarily related to dementia such as Alzheimer's disease and 2. Conditions causing problems with movements such as Parkinson's. The most widely known neurodegenerative diseases include Alzheimer (or Alzheimer's) disease along with its precursor mild cognitive impairment (MCI), Parkinson's disease (including Parkinson's disease dementia), and multiple sclerosis and a host of others. Less well-known neurodegenerative diseases include dozens of names in a comprehensive listing found at the web site of the National Institute of Neurological Disorders and Stroke (NINDS) of the National Institutes of Health (NIH) of the United States. It is understood that such diseases often go by more than one name and that a nosology may oversimplify pathologies that occur in combination or that are not archetypical or standard. Certain other disorders, such as postoperative cognitive dysfunction; described only recently, and they too may involve neurodegeneration after anesthesia and surgery. Other disorders such as epilepsy may not be primarily neurodegenerative, but at a particular point in the progression of the disorder, it might involve nerve degeneration.

Despite the fact that at least some aspect of the pathology of each of the neurodegenerative diseases mentioned above is different, their pathologies and symptoms that they have in common often make it possible to treat them with similar therapeutic agents and methods. Hence, the methods described herein can be used with selected multiple therapeutic agents as described, to treat the majority of these neurodegenerative diseases. Many publications describe features that neurodegenerative diseases have in common (Dale E. Bredesen, Rammohan V. Rao and Patrick Mehlen. Cell death in the nervous system. Nature 443 (2006): 796-802; Christian Haass. Initiation and propagation of neurodegeneration. Nature Medicine 16 (2010): 1201-1204; Michael T. Lin and M. Flint Beal. Mitochondrial dysfunction and oxidative stress in neurodegenerative diseases. Nature 443 (2006) 787-795).

The AD disease symptoms can include confusion, irritability, aggression, mood swings, trouble with language, and long-term memory loss. The sufferer often withdraws from family and society. AD is a degenerative incurable disease that the sufferer relies on others for assistance and care. The caregiver is usually one of the family members, a spouse, or close relatives, placing a great burden on them, and is one of the costliest diseases to the society and family.

The cause and progression of Alzheimer's disease is not well understood. Research shows that the disease is associated with plaques and tangles in the brain. Current treatments only help with the symptoms of the disease. There are no available treatments to stop or reverse the progression of the disease. As of 2008, more than 500 clinical trials have been conducted to find ways to treat the disease, but it is unknown if any of the tested treatments will work. Mental stimulation, exercise, NSAID intake, and a balanced diet suggested as possible ways to delay symptoms in healthy older individuals. However, they are not proven as effective treatments once the symptoms develop.

The course of the disease divided into four stages, with progressive patterns of cognitive and functional impairments. 1. Pre-dementia; 2. Mild early Start of the disease; 3. Moderate progressive deterioration; 4. Severe or advanced—the last stage in which a person is completely dependent and bed ridden.

Alzheimer's disease is characterized by the accumulation of neurofibrillary tangles (tau—τ—protein) and neuritic plaques (amyloid β) in the brain affecting especially the degeneration of neurons in the olfactory bulb and its connected brain structures. They are entorihinal cortex (EC), the hippocampal formation, amygdaloid nuclei, nucleus basalis of Meynert, locus ceruleus, and the brainstem raphe nuclei all of which project to the olfactory bulb (FIG. 14). The degenerative changes result in the loss of memory and cognitive function. There is a major loss of cortical and hippocampal choline acetyltransferase activity and degeneration of the basal forebrain cholinergic neurons. Loss of smell in Alzheimer's is due to necrosis and/or apoptosis of olfactory neurons, olfactory bulbs, olfactory tracts, the pre-pyriform cortex and the entorihinal cortex.

Etiology and Neuro-pathophysiology: The cause for most Alzheimer's cases is unknown. The amyloid hypothesis postulated that amyloid beta (Aβ) deposits are the essential cause of the disease. Also APOE4, the major genetic risk factor for AD, leads to excess amyloid buildup in the brain before AD symptoms arise. Thus, Aβ deposition precedes clinical AD. Interestingly, an experimental vaccine found to clear the amyloid plaques in early human trials, but it did not have any significant effect on dementia. Studies showed that a close relative of the beta-amyloid protein, and not necessarily the beta-amyloid itself, may be a major culprit in the disease. A 2004 study found that deposition of amyloid plaques does not correlate with neuronal loss and memory loss. This observation supports the tau hypothesis; the theory and proposal that tau protein abnormalities initiate the disease cascade. Eventually, they form neurofibrillary tangles inside nerve cell bodies resulting in the microtubules' disintegration, collapsing the neuron's transport system, causing malfunctions in biochemical communication between neurons and later in the death of the cells. Herpes simplex virus type 1 is proposed to play a causative role in people carrying the susceptible versions of the ApoE gene. Another hypothesis asserts demyelination in the aged leads to axonal transport disruptions, leading to loss of neurons. Iron released during myelin breakdown and its vascular complex has been hypothesized, and implicated as a causative factor. I do believe that the disruption of BV with release of iron from the hemoglobin around the myelin and neuropil, resulting in the iron catalyzed hydrogen peroxide called Fenton's reaction leads to generation of reactive oxygen species (ROS) during these demyelization episodes that can have an adverse effect on the neurons resulting in their apoptosis resulting in Alzheimer's.

Interestingly, the AD individuals display 70% loss of locus coeruleus cells that provide norepinephrine. Locus coeruleus cells are located in the pons, projects and innervate spinal cord, the brain stem, cerebellum, hypothalamus, the thalamic relay nuclei, the amygdala, the basal telencephalon, and the cortex. The norepinephrine from the LC has an excitatory effect on most of the brain, mediating arousal and priming the brain's neurons activated by stimuli. The norepinephrine from this nucleus stimulates microglia to suppress Aβ-induced production of cytokines and their phagocytosis of Aβ suggesting degeneration of the locus ceruleus might be responsible for increased Aβ deposition in AD brains initially. This nucleus in the pons (part of the brainstem) is involved with physiological responses to stress and panic, and is the principal site for brain synthesis of norepinephrine (noradrenalin) besides the adrenal glands.

There is no absolute diagnosis for Alzheimer's Disease to date and there is great clinical need for developing a sensitive non-invasive diagnostic. Diagnosis and monitoring of Alzheimer's disease patients is critical for assessing severity of progression to respond with the appropriate preventative care. During the onset of Alzheimer's disease, timely intervention could be life-saving. A comprehensive imaging modality for assessing Alzheimer's disease remains a significant unmet clinical need.

In vivo measurements of dopamine activity are used for understanding how this key neuromodulator contributes to cognition, neurodevelopment, aging, and neuropsychiatric disease in humans. In medicine, such measurements can result in objective biomarkers that predict clinical outcomes, including Alzheimer's disease, ideally by using procedures that capture the underlying pathophysiology while being easy to acquire in clinical settings. The locus coeruleus (LC), the primary site of noradrenaline neurons in the human brain, begins to degenerate during early stages of Alzheimer's disease (AD) and there is evidence that it is the first brain region to accumulate hyperphosphorylated tau proteins at Braak stage 0. Although this structure is heavily studied in the context of AD, much remains unclear regarding the timing of LC changes and their correlation to characteristic aspects of AD pathophysiology and clinical features.

The central noradrenergic system plays a key role in arousal and consolidation of emotional memory. The locus coeruleus (LC), the primary site of noradrenergic neurons in the brain, has a topographic pattern of projections, with the caudal extent of the LC sending descending projections modulating autonomic signalling. Dysregulation of the noradrenergic system has been implicated in theoretical accounts of PTSD, particularly in regard to symptoms of hyperarousal and in major depressive disorder (MDD). Despite a strong theoretical foundation, understanding of noradrenergic dysfunction in PTSD and MDD is incomplete, impeding research into novel treatments targeting this system in PTSD. Recent work has pioneered the use of a specialized neuroimaging technique, Neuromelanin-Sensitive MRI (NM-MRI), a non-invasive method to probe the function of the human noradrenergic system in vivo by examining signal contrast in the LC. NM-MRI signal here has been positively related to emotional memory performance and autonomic function (indexed by salivary alpha amylase or heart rate variability) but it has yet to be investigated in individuals with PTSD. On the other hand, low LC NM-MRI signal has been observed in major depressive disorder. We hypothesized that hyperarousal symptoms of PTSD and MDD would be positively correlated to NM-MRI signal in the caudal LC.

Neuromelanin (“NM”) is a dark pigment synthesized via iron-dependent oxidation of cytosolic dopamine and subsequent relation with proteins and lipids in midbrain dopamine neurons. NM pigment accumulates inside specific autophagic organelles, which contain NM-iron complexes, along with lipids and various proteins. NM-containing organelles accumulate gradually over the lifespan in the soma of dopamine neurons in the substantia nigra (“SN”) a nucleus that owes its name to its dark appearance due to the high concentration of NM, and are only cleared from tissue following cell death through the action of microglia. Given that NM-iron complexes are paramagnetic, they can be imaged using MRI. A family of MRI sequences known as NM-MRI captures groups of neurons with high NM content, such as those in the SN, as hyperintense regions.

Thus, it would be beneficial to provide a system, process, method and computer-accessible medium for neuromelanin-sensitive MRI which can overcome the deficiencies described above. Different neurological and psychiatric diseases have been associated with neuromelanin changes in two main regions, the substantia nigra pars compacta (SNc) and the locus coeruleus (LC). Differentiating between different disorders with similar clinical presentations solely based on presenting symptoms is difficult because the symptoms often overlap between related conditions.

There are currently no FDA cleared software as medical devices for the measurement of NM in the SN or LC.

The unmet medical need addressed here is the ability to differentiate between related disorders such as Parkinson's disease, multiple system atrophy, and progressive supranuclear palsy as well as different dementias such as Alzheimer's disease and dementia with Lewy bodies. The increase in ability to differentiate between related disorders will drive improved patient outcomes.

### SUMMARY

Provided herein are, inter alia, methods for determining the presence of Alzheimer's disease in a subject and determining the change in the concentration of neuromelanin in the subject with time. The concentration of neuromelanin may change as the result of the regular course of Alzheimer's disease or as a result of therapeutic intervention. In a first aspect, there is provided a method of determining whether a change in the concentration of neuromelanin occurs over time in the brain of a subject. In a preferred embodiment, the subject is an Alzheimer's disease patient. The method includes obtaining a first neuromelanin magnetic resonance image of the subject at a first time point. Subsequently a second neuromelanin magnetic resonance image is obtained at a second time point. The first magnetic resonance image is compared to the second magnetic resonance image, thereby determining whether a change in the concentration of neuromelanin occurred between the first time point and the second time point.

The present disclosure describes the combined use of two fully automated algorithms to measure neuromelanin (NM) concentrations and volumes in two different brain regions (the SNc and the LC) to improve the ability to differentiate between related disorders. The voxel-based analysis algorithm (previously described in WO 2020/077098 and WO 2021/034770, the entire contents of each of which are incorporated herein by reference) is used to measure NM in the SNc. However, since the LC is much smaller, and may not be as well suited to voxel-based analysis on a 3T MRI (the most commonly available scanners in the clinic) a new algorithm was invented at University of Ottawa to measure NM in the LC. This LC algorithm is termed the segmented based analysis algorithm. This disclosure describes the combination of the two algorithms together in a software package that can be used to aid in the diagnosis and differentiation of neuropsychiatric disorders that are difficult to differentiate between based on symptoms alone.

In the present disclosure, the software uses two algorithms. The voxel-based analysis algorithm is used to measure NM in the SNc and the segmented based analysis algorithm is used to measure NM changes in the LC. The software reports the NM levels and volumes in both brain regions to the physician. The combination of these two algorithms together increases the ability to differentiate between related neurological conditions. Their inclusion in a fully automated software allows the potential for their widespread use in the clinic.

In one embodiment, the present disclosure is directed to a method of diagnosing, Alzheimer's disease in a subject comprising:
(i) performing a Neuromelanin-Magnetic Resonance Imaging (NM-MRI) scan, measuring a level of neuromelanin,
(ii) comparing the level of neuromelanin to previous scans and/or reference values, and
(iii) providing a diagnosis of Alzheimer's disease.

In one embodiment, the present disclosure is directed to a method of monitoring progression of Alzheimer's disease in a subject comprising:
(i) performing a Neuromelanin-Magnetic Resonance Imaging (NM-MRI) scan, measuring a level of neuromelanin,
(ii) comparing the level of neuromelanin to previous scans and/or reference values, and
(iii) determining the progression of Alzheimer's disease.

In one embodiment, the present disclosure is directed to a method of providing a prognosis of Alzheimer's disease in a subject comprising:
(i) performing a Neuromelanin-Magnetic Resonance Imaging (NM-MRI) scan, measuring a level of neuromelanin,
(ii) comparing the level of neuromelanin to previous scans and/or reference values, and
(iii) optionally providing a prognosis of Alzheimer's disease.

In one embodiment, the present disclosure is directed to a method of monitoring treatment of Alzheimer's disease in a subject comprising:
(i) performing a Neuromelanin-Magnetic Resonance Imaging (NM-MRI) scan, measuring a level of neuromelanin,
(ii) comparing the level of neuromelanin to previous scans and/or reference values, and
(iii) assessing the effect of treatment of Alzheimer's disease.

In one embodiment, the present disclosure is directed to determining a first signal intensity from a first neuromelanin magnetic resonance image and determining a second signal intensity from a second neuromelanin magnetic resonance image, and comparing the first magnetic resonance image to said second magnetic resonance image comprises comparing the first signal intensity to the second signal intensity.

In one embodiment, the control is a level of neuromelanin present at approximately the same levels in a population of subjects, or said standard control is approximately the average level of neuromelanin present in a population of subjects.

In one embodiment, a neuromelanin gradient phantom is used to measure the level, signal and/or concentration of neuromelanin.

In one embodiment, a neuromelanin phantom concentration gradient is scanned about once per patient, about once an hour, about once a day, about once a week, or about once a month.

In one embodiment, the neuromelanin phantom gradient is scanned daily.

In one embodiment, the neuromelanin phantom gradient is scanned with each patient.

In one embodiment, the present disclosure is directed to a method of assessing the neuromelanin in a subject comprising:
performing an Neuromelanin-Magnetic Resonance Imaging (NM-MRI) scan on the subject;
acquiring a neuromelanin dataset from the NM-MRI scan;
optionally encrypting the neuromelanin dataset;
uploading the neuromelanin dataset to a remote server;
optionally decrypting the dataset;
   performing an analysis of the neuromelanin dataset, wherein the analysis comprises one or more of:
(i) comparing the neuromelanin dataset with one or more previously acquired neuromelanin datasets from the said subject
(ii) comparing the neuromelanin dataset with a control dataset;
(iii) comparing the neuromelanin dataset with one or more previously acquired neuromelanin datasets from different subjects;
generating a report comprising the neuromelanin analysis;
optionally encrypting the report;
uploading the report to remote server;
optionally decrypting the report.

In one embodiment, the disclosure is directed to an in vivo method of determining the progression of Alzheimer's disease over time in a subject, said method comprising:
(i) obtaining a first neuromelanin magnetic resonance image at a first time point;
(ii) after step (i) comparing the first neuromelanin magnetic resonance image to an age matched control;
(iii) determining the level, signal and/or concentration of neuromelanin occurred between said first time point and said second time point.

In one embodiment, the disclosure is directed to an in vivo method of diagnosing Alzheimer's disease, said method comprising:
(i) obtaining a first neuromelanin magnetic resonance image at a first time point;
(ii) after step (i), obtaining a second neuromelanin magnetic resonance image at a second time point;
(iii) comparing the first neuromelanin magnetic resonance image to said second neuromelanin magnetic resonance image thereby determining whether a change in one or more of the level, signal or concentration of neuromelanin occurred between said first time point and said second time point.

In one embodiment, the disclosure is directed to a method of providing a treatment regimen to a patient comprising performing the NM-MRI scan, acquiring NM signal from the NM-MRI scan in a region of interest, comparing the NM signal from the NM-MRI scan in a region of interest data to age matched database numbers, if the NM signal is less than a pre-determined value, administering a corresponding treatment regimen.

In one embodiment, the subject displays symptoms of Alzheimer's disease.

In one embodiment the patient suffers from a disorder commonly misdiagnosed as Alzheimer's disease.

In one embodiment, the NM-MRI scan and analysis distinguishes between Alzheimer's disease and Parkinson's disease. In one embodiment, the NM-MRI scan and analysis distinguishes between and can separately identify related disorders (e.g. dementia with Lewy Bodies). In one embodiment, the NM-MRI scan and analysis can monitor the progression of, monitor the treatment of, and provide a prognosis for disorders related to Alzheimer's disease.

In one embodiment, the present disclosure is directed to a method of determining if a subject has or is at risk of developing Alzheimer's disease, the method comprising analyzing one or more Neuromelanin-Magnetic Resonance Imaging (NM-MRI) scans of the subject's brain region of interest, wherein the analyzing comprises:
receiving imaging information of the brain region of interest; and
determining a NM concentration in the brain region of interest using segmented analysis based on the imaging information;
wherein the determining if a subject has or is at risk of developing Alzheimer's disease comprises:
(1) if the one or more NM-MRI scans has a decreased NM signal compared to a one or more control scans without Alzheimer's disease then the subject has or is at risk of developing Alzheimer's disease; or
(2) if the one or more NM-MRI scans has a NM signal comparable to the signal of a one or more control scans without Alzheimer's disease then the subject does not have or is not at risk of developing Alzheimer's disease.

In one embodiment, the present disclosure is directed to a method of treating a subject with Alzheimer's disease, the method comprising analyzing Neuromelanin-Magnetic Resonance Imaging (NM-MRI) scans of the subject's brain region of interest, wherein the analyzing comprises:
receiving imaging information of the brain region of interest at a first time point;
receiving imaging information of the brain region of interest at a second time point;
determining a NM concentration at the first and second time points in the brain region of interest using segmented analysis based on the imaging information; and
comparing the NM concentration at the first time point to the second time point,
wherein the treatment method further comprises:
(1) administering one or more of levodopa and carbidopa if the NM-MRI scan at the second time point has a decreased NM signal compared to the NM signal at the first time point; or
(2) withhold administering one or more of levodopa and carbidopa if the NM-MRI scan at the second time point has an increased NM signal compared to the NM signal at the first time point.

In one embodiment, the subject exhibits one or more symptom of Alzheimer's disease.

In one embodiment, the method provides a diagnosis of Alzheimer's disease before symptoms present clinically.

In one embodiment, the NM-MRI method distinguishes between Alzheimer's disease and Parkinson's disease.

In one embodiment, the NM-MRI method diagnoses the patient as having Alzheimer's disease or as not having Alzheimer's disease; and indicates the diagnosis to a user via a user interface.

In one embodiment, the analysis is a segmented analysis.

In one embodiment, the segmented analysis comprises determining at least one topographical pattern within the brain region of interest.

In one embodiment, the method further comprises a calculation using a value that represents a volume of a neuromelanin voxel or segment.

In one embodiment, the segmented region of interest is the locus coeruleus.

In one embodiment, the disclosure is directed to a diagnostic system for providing diagnostic information for Alzheimer's disease, the diagnostic system comprising:
an MRI system configured to generate and acquire a neuromelanin sensitive MRI scan along with a neuromelanin data series for a voxel or segment located within a region of interest in a subject's brain;
a signal processor configured to process the series of neuromelanin data to produce a processed neuromelanin MRI spectrum; and
a diagnostic processor configured to process the processed neuromelanin MRI spectrum to:
extract a measurement from the region of interest corresponding with neuromelanin at a time point,
compare the measurement to one or more control measurements acquired prior to the time point;
provide a diagnosis of Alzheimer's disease if the measurement is more than about 25% less than the control measurement.

In one aspect, the present disclosure is directed to a method for determining whether brain tissue in a subject contains an abnormal level of neuromelanin. The method includes detecting a level neuromelanin in the tissue. The level of neuromelanin is compared to a standard control. If a lower level of neuromelanin is detected relative to the standard control, this indicates Alzheimer's disease.

In one embodiment, there is provided a method for determining whether a Alzheimer's disease therapy administered to a subject is effective. The method includes a step of detecting a level of endogenous neuromelanin in the tissue at a first time point. In a subsequent step, a therapy is administered to the subject. A level of neuromelanin in the tissue is then determined at a second time point. Thereafter, the level of neuromelanin at the first time point is compared to the level of neuromelanin at the second time point. A higher level or a constant level of neuromelanin at the second time point relative to the first time point indicates that the therapy was effective. Alternatively, a lower level neuromelanin at the second time point relative to the first time point indicates that the therapy administered to the subject was ineffective.

In one embodiment, there is provided a method for treating a patient with Alzheimer's disease. In one embodiment, the method comprises administering to a patient an initial amount of an Alzheimer's disease treatment. In one embodiment, the method comprises monitoring the neuromelanin concentration in a region of interest in the patient's brain and assessing treatment-related adverse events over an initial treatment period. In one embodiment, if, during the initial treatment period, the patient exhibits one or more of
i) decreased neuromelanin concentration in the region of interest in the patient's brain; and
ii) no treatment associated adverse or side effects;
then increasing the dose of the Alzheimer's disease treatment in a subsequent treatment period;
wherein the treatment results in an improvement in Alzheimer's disease symptoms in the patient.

In one embodiment, the treatment method, includes the following step:
repeating steps a)-c) until the patient fails to exhibit one or more of i)-ii) in step c).

In one aspect, the present disclosure is directed to a method of diagnosing, determining the progression over time of, or providing a prognosis of a neurological disorder in a subject, said method comprising:
(i) obtaining a first Neuromelanin Magnetic Resonance Imaging (NM-MRI) scan at a first time point;
(ii) after step (i), obtaining a second NM-MRI scan at a second time point;
(iii) performing a segmented-based algorithm analysis to determine the level, concentration and/or volume of neuromelanin (NM) in the locus coeruleus (LC);
(iv) performing a voxel-based algorithm analysis to determine the level, concentration and/or volume of neuromelanin in the substantia nigra pars compacta (SNc);
(v) comparing the first neuromelanin magnetic resonance image to the second neuromelanin magnetic resonance image thereby determining whether a change in the level, signal and/or concentration of neuromelanin occurred between said first time point and said second time point in both the SNc with the voxel-based algorithm and the LC with the segmented based algorithm.
(vi) providing a diagnosis, progression over time, or prognosis of the neurological disorder based on the difference in the level of NM in the SNc between the first and second scans and the difference in the level of NM in the LC between the first and second scans.

In one aspect, the present disclosure is directed to an in vivo method of selecting a treatment regimen for the prevention or treatment of a neurological disorder in a subject, said method comprising:
(i) obtaining a first Neuromelanin Magnetic Resonance Imaging (NM-MRI) scan at a first time point;
(ii) after step (i), obtaining a second NM-MRI scan at a second time point;
(iii) performing a segmented-based algorithm analysis and determining the level, concentration and/or volume of neuromelanin (NM) in the locus coeruleus (LC);
(iv) performing a voxel-based algorithm analysis and determining the level, concentration and/or volume of neuromelanin in the substantia nigra pars compacta (SNc);
(v) comparing the first neuromelanin magnetic resonance image to the second neuromelanin magnetic resonance image thereby determining whether a change in the level, signal and/or concentration of neuromelanin occurred between said first time point and said second time point in both the SNc with the voxel-based algorithm and the LC with the segmented based algorithm;
(vi) providing a diagnosis, progression over time, or prognosis of the neurological disorder based on the difference in the level of NM in the SNc between the first and second scans and the difference in the level of NM in the LC between the first and second scans.
(vi) administering the treatment regimen corresponding with the determined neurological disorder.

In one aspect, the present disclosure is directed to a method for distinguishing between motor diseases with similarly presenting symptoms comprising:
(i) performing an examination to determine a Unified Parkinson's Disease Rating Scale score;
(ii) obtaining a first Neuromelanin-Magnetic Resonance Imaging (NM-MRI) scan at a first time point;
(iii) after steps (i) and (ii), obtaining a second NM-MRI scan at a second time point;
(iv) performing a voxel-based analysis and determining the concentration and/or volume of NM in the SNc;
(v) performing a segmented based analysis and determining the concentration and/or volume of NM in the LC;
(vi) comparing the first neuromelanin magnetic resonance image to the second neuromelanin magnetic resonance image thereby determining whether a change in the level, signal and/or concentration of neuromelanin occurred between said first time point and said second time point in both the SNc and LC;
(vi) providing a diagnosis, progression over time, or prognosis of the neurological disorder based on the difference in the level of NM in the SNc between the first and second scans and the difference in the level of NM in the LC between the first and second scans.

In one aspect, the present disclosure is directed to a method of diagnosing a patient with a neurological disorder, said method comprising:
(i) measuring a concentration and/or volume of neuromelanin in the SNc using a voxel-based analysis method and measuring a concentration and/or volume of neuromelanin in the LC using a segmented based analysis method;
(ii) comparing the level of neuromelanin in the SNc to a standard control level of neuromelanin in the SNc and comparing the level of neuromelanin in the LC to a standard control level of neuromelanin in the LC,
(iii) providing a diagnosis of the neurological condition if the magnitude or ratio of SNc and LC neuromelanin is lower or higher for each of their respective regions relative to the standard control.

In one aspect, the present disclosure is directed to a method of diagnosing a patient with a neurological disorder, said method comprising:
(i) measuring a concentration and/or volume of neuromelanin in the SNc using a voxel-based analysis method and measuring a concentration and/or volume of neuromelanin in the LC using a segmented based analysis method;
(ii) comparing the level of neuromelanin in the SNc to a standard control level of neuromelanin in the SNc and comparing the level of neuromelanin in the LC to a standard control level of neuromelanin in the LC,
(iii) providing a diagnosis of the neurological condition if the magnitude or ratio of SNc and LC neuromelanin is lower or higher for each of their respective regions relative to the standard control according to pre-determined values.

In one embodiment, the methods described herein are used with a second imaging method, wherein the second imaging method is selected from the group consisting of positron emission tomography (PET), tau-PET, structural MRI, comprises functional MRI (fMRI), blood oxygen level dependent (BOLD) fMRI, iron sensitive MRI, quantitative susceptibility mapping (QSM), diffusion tensor imaging DTI, and single photon emission computed tomography (SPECT), DaTscan and DaTquant.

In one embodiment, the methods described herein are used with a second imaging method, wherein the second imaging method is Positron Emission Tomography (PET).

In one embodiment, the methods described herein are used with a second imaging method, wherein the second imaging method is structural MRI.

In one embodiment, the methods described herein are used with a second imaging method, wherein the second imaging method is functional MRI (fMRI).

In one embodiment, the methods described herein are used with a second imaging method, wherein the second imaging method is blood oxygen level dependent (BOLD) fMRI.

In one embodiment, the methods described herein focuses on the neuromelanin level, concentration, volume, or pattern within symptom-specific and/or disease-specific voxels in the SNc.

In one embodiment, the methods described herein focuses on the neuromelanin level, concentration, volume, or pattern within symptom-specific and/or disease-specific segments in the LC.

In one embodiment, the methods described herein focuses on the neuromelanin level, concentration, volume, or pattern within symptom-specific voxels and/or disease-specific in the SNc and the neuromelanin level, concentration, volume, or pattern within symptom-specific and/or disease-specific segments in the LC.

In one embodiment, the methods described herein focuses on the neuromelanin level, concentration, volume, or pattern within the SNc and the neuromelanin level, concentration, volume, or pattern within symptom-specific and/or disease-specific segments in the LC.

In one embodiment, the methods described herein focuses on the neuromelanin level, concentration, volume, or pattern within symptom specific and/or disease-specific voxels in the SNc and the neuromelanin level, concentration, volume, or pattern within the LC.

In one embodiment, the methods discussed herein are directed to one or more neurological condition.

In one embodiment, the methods discussed herein are directed to one or more neurological condition, wherein the neurological condition is selected from schizophrenia, cocaine use disorder, Parkinson's disease, Alzheimer's disease without neuropsychiatric symptoms, neuropsychiatric symptoms of Alzheimer's disease, major depressive disorder, and/or post-traumatic stress disorder.

### BRIEF DESCRIPTION OF THE FIGURES

The accompanying drawings, which are included to provide a further understanding of the present disclosure, are incorporated in and constitute a part of this specification, illustrate aspects of the present disclosure and, together with the detailed description, serve to explain the principles of the present disclosure. The patent or application file contains at least one drawing executed in color. Copies of this patent or patent application publication with color drawing(s) will be provided by the Office upon request and payment of the necessary fee.

FIG. 1 shows Neuroimaging measures. Top: PET imaging measures of tau load using radiotracer [¹⁸F]MK6420 (left) and β-amyloid load using [¹⁸F]AZD4694 (right) in representative cognitively normal (CN) and Alzheimer's disease (AD) participants. Bottom: Imaging the locus coeruleus (LC). Bottom-left: NM-MRI image obtained in vivo from a CN older adult. Bottom-middle: magnified view of the pons from this participant and from a representative AD patient. This non-invasive procedure clearly delineates the LC as hyperintense voxels (yellow arrows). In AD, LC degeneration begins in the early stages of illness, causing visible reduction in LC NM-MRI signal. Bottom-right: 3D structure of human LC revealed by computer reconstruction showing distribution of noradrenergic LC cells (orange) based on post-mortem cell counts

FIG. 2 shows a prediction of neuropsychiatric symptom severity in n=73 cognitively impaired older adults based on LC NM-MRI (left), tau load (middle), or β-amyloid load (right). NPS severity was adjusted based on covariates age, sex, dementia severity, and PET measures (LC plot) or LC signal (tau and amyloid plots). All measures significantly predicted MBI total score (Pearson r=0.37, 0.44, 0.40 respectively) and MBI impulse dyscontrol subdomain score (plots not shown, r=0.35, 0.30, 0.29, respectively).

FIG. 3 shows NM-MRI images acquired at 7 Tesla and 3 Tesla from representative subjects. Yellow arrows point to the LC. Compared to 3T, ultra-high field strength (7T) allows enhanced in-plane resolution (0.7×0.7 mm at 3T vs 0.4×0.4 mm at 7T; axial view) and thinner slices (1.8 vs 1.0 mm; coronal view); therefore, voxel volume is 5.5 times smaller at 7T. Lower resolution causes noise in the LC NM-MRI signal due to partial volume effects where single voxels combine LC and non-LC tissue. For this reason, ultra-high field NM-MRI is preferred to measure the signal from this small structure

FIG. 4 shows the measurement of the LC NM-MRI signal. A. NM-MRI visualization template created by averaging many NM-MRI images in MNI space. B, C. Magnified views of template with a manually traced over-inclusive mask of the LC overlaid. This mask is divided into 4 rostro-caudal segments (color-coded in B). D. A representative subject's NM-MRI image showing the pons in native space. The overinclusive LC mask is converted from MNI space to native space to create a search space (yellow) wherein to locate the LC. E. The LC (yellow) is identified bilaterally as the brightest cluster of 4 adjacent voxels within the search space. F. The contrast to noise ratio (CNR) is calculated for all voxels relative to the signal in a reference region containing no NM (white circle). Averaging the CNR values from all LC voxels in a rostro-caudal segment yields the LC NM-MRI signal. The mid-rostral segment (yellow) showed the most pronounced degeneration in AD and the signal here is used for all analyses.

FIG. 5 shows the relationship of LC NM-MRI signal to Braak stage and dementia severity. Left: schematic representation of the LC in coronal view showing subregional pattern of NM-MRI signal loss in tau positive individuals. The LC was divided into 5 segments (each 3 mm long) on the left and right sides. Tau status was divided into 3 levels (tau negative, Braak region 1 positive, Braak region 3 positive). The LC segments are color-coded based on the relationship of NM-MRI signal in the segment to tau status (t-statistic derived from a robust linear regression controlling for age and sex). The strongest relationship was seen in the middle LC segment (MNI space z coordinate=−22 to −25; encircled in yellow and matching the yellow LC segment shown in FIG. 1). Bilateral LC NM-signal from this segment was the NM-MRI metric selected for all subsequent analyses. Middle: scatterplot showing LC NM-MRI signal in all study groups. Braak 3 positive cases (dark red) showed reduced signal relative to tau negative cases and Braak 1 positive cases. Error bars represent standard error of the mean. Right: scatterplots showing correlation of LC NM-MRI signal to cognitive impairment (errors on the MMSE, top) and dementia stage (CDR score, bottom). L: left, R: right, CN−: cognitively normal tau negative individuals, CI+: cognitively impaired Braak 1 positive individuals, CI++: cognitively impaired Braak 3 positive individuals, MMSE: Mini Mental State Exam, CDR: Clinical Dementia Rating Scale.

FIG. 6 shows the voxel wise correlation of LC NM-MRI signal to [18F]MK-6240 uptake throughout the brain.

FIG. 7 shows the measurement of LC NM-MRI signal. Left: visualization template in MNI space created by averaging the spatially normalized NM-MRI images from all participants. Middle: magnified views of the visualization template with the over-inclusive LC mask overlaid. This mask was manually traced on the visualization template over the hyperintense region surrounding the LC and divided into 5 rostrocaudal segments (displayed in different colors), each spanning 3 mm in the z axis. Top-right: unprocessed NM-MRI image showing the pons of a representative individual; the central pons reference region is encircled in white. Contrast-to-noise ratio for all voxels was calculated relative to signal from this region. Bottom-right: segmentation of the LC in native space. The over-inclusive LC mask (yellow) was deformed from MNI space to native space to provide a search space wherein the LC was identified on left and right sides as the brightest 4 adjacent voxels. To minimize partial volume effects, only the brightest 1 of the 4 voxels was retained for each side and slice and the LC NM-MRI signal was calculated by averaging CNR values from these voxels for each of the 5 segments.

FIG. 8 shows a positive correlation between caudal LC NM-MRI signal and CAPS-5 hyperarousal symptom severity.

FIG. 9 shows a negative correlation between LC NM-MRI signal and BDI depression severity.

FIG. 10 shows LC signal in PTSD patients and healthy individuals. There was a significant increase in caudal LC signal in the PTSD group controlling for age (t34=2.08, p=0.046, cohen's d=0.71; linear regression controlling for age).

FIG. 11 shows the relationship of clinical and physiological measures of hyperarousal to LC NM-MRI signal. Left: Greater LC NM-MRI signal was significantly related to more severe symptoms of hyperarousal in 24 Canadian Armed Forces veterans with a history of operational deployment (r=0.52, p=0.019). Right: Preliminary data suggests a positive correlation between LC NM-MRI signal and skin conductance response during the fMRI fear conditioning procedure (phasic max, conditioned stimulus-unconditioned stimulus) in 7 young healthy individuals (age- and diagnosis-related increase in NM signal likely explains the lower LC NM-MRI signal values compared to the plot at left).

FIG. 12 shows that LC localization via NM-MRI supports fMRI analysis. Due to its small size, it is not recommended to examine activity of the LC using standard methods of BOLD fMRI preprocessing and analysis. Recent work has demonstrated an improved method that conducts first-level fMRI analysis in native space with no smoothing and leverages the NM-MRI signal to provide a personalized LC localizer [46, 47]. We employed this approach in a single subject with PTSD and examined functional connectivity of the LC. At rest (top), we observed a pattern of functional connectivity very similar to prior report [46], centered around the LC (white) and including structures within the brainstem and cerebellum. After exposure to personalized trauma words in this same individual (bottom), the connectivity of the LC increased with many structures known to project to or from the LC including hypothalamus, hippocampus, and cerebral cortex. In the current proposal the fMRI paradigm consists of fear conditioning, not trauma evocation as shown here; nevertheless, these results demonstrate the feasibility of the approach to investigate functional activity of the LC using BOLD fMRI.

FIG. 13 shows SNc and LC masks. The software automatically applies the custom SN mask to the SNc to select the region for the voxel based algorithm and the custom LC mask to the LC to select the region for the segmented algorithm.

FIG. 14 shows the application of the voxel based and segmented based algorithms to measure NM in patients with Parkinson's disease. The voxel based algorithm shows there are significant changes in the SNc compared to healthy controls (left panel). The segmented based algorithm shows there are no significant changes in the LC compared to healthy controls (right panel).

FIG. 15 shows the application of the voxel based and segmented based algorithms to measure NM in patients with mild cognitive impairment (MCI) and Alzheimer's disease (AD). The voxel based algorithm shows there are no significant changes in the SNc compared to healthy controls (left panel). The segmented based algorithm shows there are significant changes in the LC compared to healthy controls (right panel).

FIG. 16 shows the application of the voxel based and segmented based algorithms to measure the neuropsychiatric symptoms NM in with Alzheimer's disease (AD). The segmented based algorithm shows there are significant increases in NM in the LC compared to healthy controls (left panel). The voxel based algorithm shows there are significant decreases in NM compared to healthy controls in the SNc(right panel).

FIG. 17 shows the application of the voxel based and segmented based algorithms to measure NM in patients with Schizophrenia. The voxel based algorithm shows there are significant changes in the SNc compared to healthy controls (left panel). The segmented based algorithm shows there are no significant changes in the LC compared to healthy controls (right panel).

FIG. 18 shows the application of the voxel based and segmented based algorithms to Post Traumatic Stress Disorder. The voxel based algorithm shows there are no significant association of disease severity with NM levels in the SNc compared to healthy controls (left panel). The segmented based algorithm shows there are significant changes in the LC compared to healthy controls and that the increase NM levels are significantly associated with disease severity (right panel).

FIG. 19 shows the application of the voxel based and segmented based algorithms to patients with depression. The voxel based algorithm shows there is are no significant association of disease severity with NM levels in the SNc compared to healthy controls (left panel). The segmented based algorithm shows there is a trend toward decreasing NM levels with increasing disease severity in the LC compared to healthy controls (right panel).

FIG. 20 shows the application of the voxel based and segmented based algorithms to cocaine use disorder. The voxel based algorithm shows that increased NM in the SNc is significantly associated with cocaine use disorder compared to healthy controls (left panel). The segmented based algorithm shows there is a trend toward decreasing NM in the LC compared to healthy controls (right panel).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### Definitions

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description is taken with the drawings making apparent to those skilled in the art how the forms of the present disclosure may be embodied in practice.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range. Moreover, the terminology at least includes the stated number, e.g., “at least 50” includes 50.

The term “MR” refers to magnetic resonance and is the physical principle upon which a variety of experimental procedures known in the art and/or described herein are based, including MRI (“magnetic resonance imaging”), MRS (“magnetic resonance spectroscopy”) and the like. The term neuromelanin-sensitive MRI or neuromelanin-MRI refer to the use of MRI in the study of neuromelanin in the brain. Herein the general term magnetic resonance image, magnetic resonance imaging or MRI encompasses neuromelanin-sensitive variants.

As used herein, the term “NM-MRI” and similar nomenclature refers to each the MRI scan and corresponding voxel wise analysis independently, both as separate and together.

The terms “T1” and “T2” used herein refer to the conventional meanings well known in the art (i.e., “spin-lattice relaxation time,” and “spin —spin relaxation time,” respectively).

The term “T1-weighted” in the context of MRI images refers to an image made with pulse spin echo or inversion recovery sequence, having appropriately shortened TR and TE, which as known in the art can demonstrate contrast between tissues having different T1 values. The term “TR” in this context refers to the repetition time between excitation pulses. The term “excitation pulse” is understood to refer to a 90-deg radio frequency (RF) excitation pulse. The term “TE” refers to the echo time between the excitation pulse and MR signal sampling.

The term “subject” may be a mammalian subjects such as murine, rattus, equine, bovine, ovine, canine, feline or human. In some embodiments of the methods described herein, the subject is a mouse, while in other embodiments the subject is a human. The term “patient” in this context refers to a human subject.

As used herein, the term “alleviate” is meant to describe a process by which the severity of a sign or symptom of a disorder is decreased. Importantly, a sign or symptom can be alleviated without being eliminated. In a preferred embodiment, the use of treatment methods disclosed herein leads to the elimination of a sign or symptom, however, elimination is not required. Effective dosages guided by the present disclosure are expected to decrease the severity of a sign or symptom.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. An effective amount of a pharmaceutical agent is that which provides an objectively identifiable improvement.

The term “neurological” condition is used interchangeably with “neurological disorder” and “neurological disease” and is intended to encompass the conditions/disorders known in the art, at least several of which have been enumerated herein.

As used herein, “stable” refers to measurements that are reproducible. In one embodiment, “stable neuromelanin levels” refers to serial scans where neuromelain levels remain relatively constant. In some contexts, “stable neuromelanin levels” are maintained for one or more hours, one or more days, one or more weeks, or one or more treatment cycles.

The terms “treat,” “treatment” and the like in the context of disease refer to ameliorating, suppressing, eradicating, and/or delaying the onset of the disease being treated. In some embodiments, the methods described herein are conducted with subjects in need of treatment. The terms “in need of treatment” and the like as used herein refer to a subject at risk for developing a disease, having a condition, which would be understood by those of skill in the medical or veterinary arts as likely leading to a disease, and/or actually having a disease. Alzheimer's disease treatments includes currently approved and investigative treatments. Conventional MRI lacks the spatial and quantitative data needed to predict clinical outcomes. However, the methods as discussed herein detect levels of neuromelanin in the brain that can predict clinical progression, severity, and response in Alzheimer's disease given the variance of neuromelanin in the brain or loss of neuromelanin-containing neurons.

The NM-MRI of the present disclosure can monitor the efficacy of Alzheimer's treatment. The NM-MRI of the present disclosure can determine efficacy of investigative treatments. A non-exhaustive listing of Alzheimer's treatment which may be monitored according to one embodiment of the present disclosure includes one or more of the following:

Alzheimer's disease treatments include disease-modifying therapies. These therapies aim to prevent, slow or halt the overall progression of Alzheimer's disease (PD). They target different proteins and pathways believed to play a role in the disease.

In some embodiments NM-MRI provides a method for dose titration for the treatment of Alzheimer's disease while avoiding and adverse or side effects from currently approved or investigational therapeutics. Specifically, administering a treatment while monitoring NM signals using the voxelwise approach described herein to guide the dosage regimen, it is possible to increase efficacy.

Additionally, administering a therapeutic according to a specific variable dosage regimen guided by NM-MRI, it is possible to reduce side effects which may be associated with administration. For example, administering treatments according to the specific dosage regimen guided by NM-MRI voxel analysis of the present disclosure may significantly reduce, or even completely eliminate treatment associated side effects.

In one embodiment, the region of interest are Alzheimer's disease symptom-associated voxels. The dose variation increases patient compliance, improves therapy and reduces unwanted and/or adverse effects. In certain embodiments, the therapeutic method of the disclosure provides an improved overall therapy relative to the administration of the therapeutic agents by themselves.

In certain embodiments, doses of existing therapeutic agents can be reduced or administered less frequently in using the guided intervention of the present disclosure, thereby increasing patient compliance, improving therapy and reducing unwanted or adverse effects. In one embodiment, monitoring treatment with the NM-MRI of the present disclosure allows patients to experience benefit from treatment for a longer timeframe.

Neuromelanin-sensitive MRI data may be used as a biomarker for Alzheimer's disease, or risk of developing Alzheimer's disease, severity, illness progression, treatment response, and/or clinical outcome. Neuromelanin-sensitive MRI methods meet the need for objective biomarker tracking Alzheimer's disease, severity, or risk for its development. Neuromelanin-sensitive MRI can be used as a safe alternative for invasive/radiating imaging measures (e.g., PET). Neuromelanin-sensitive MRI can also be used for monitoring of progression, which currently cannot be done given the risk of repeated exposure to radiation. Neuromelanin-sensitive MRI is non-invasive, cheaper, safer, and easier to acquire in clinical settings. It has substantially increased (5-10-fold) anatomical resolution, which allows for resolving anatomical detail within relevant brain structures.

In certain embodiments, neuromelanin sensitive magnetic resonance images are obtained periodically, for example, every 1, 2, 3, 4, 5, 6 or 7 days, every 1, 2, 3 or 4 weeks, every 1, 2, 3, 4, 5, 6, 7, 8, 9, 10, 11 or 12 months, or every 1, 2, 3, 4 or 5 years. In certain embodiments, a first magnetic resonance image is obtained prior to the appearance of symptoms. In certain embodiments, a first magnetic resonance image is obtained prior to symptoms associated with Alzheimer's disease. A second magnetic resonance image may be obtained either prior to or subsequent to the appearance of symptoms. In other embodiments, a second magnetic resonance image may be obtained 1 year after the first magnetic resonance image.

In some embodiments, the neuromelanin sensitive magnetic resonance imaging (“NM-MRI”) technique is effective at non-invasively diagnosing, measuring the effect of, and/or providing a prognosis for Alzheimer's disease.

In some embodiments, the NM-MRI technique is used as a tool for diagnosing pre-symptomatic Alzheimer's disease. In some embodiments, the NM-MRI technique is effective for distinguishing Alzheimer's disease from other neurological conditions, including but not limited to Parkinson's disease and/or dementia with lewy bodies. In other embodiments, the NM-MRI technique is effective at selecting and/or monitoring a course of treatment, optionally, such a treatment is effective at treating Alzheimer's disease.

In some embodiments, the NM-MRI technique is used as a tool for monitoring the progress of Alzheimer's disease. In some embodiments, the NM-MRI technique is effective for the longitudinal assessment of Alzheimer's disease progression.

In some embodiments the technique measures neuromelanin directly or indirectly. In other embodiments, the technique measures dopamine function directly or indirectly. In some embodiments, there is a connection between neuromelanin-sensitive MRI (NM-MRI) signal and Alzheimer's disease severity.

In some embodiments, the NM-MRI technique is capable of determining the concentrations of neuromelanin across all sections of brain tissue. In other embodiments, the NM-MRI technique is capable of determining regional concentrations of neuromelanin. In other embodiments, the NM-MRI technique is capable of determining regional levels of neuromelanin. In other embodiments, the NM-MRI technique is capable of determining regional signal intensity of neuromelanin.

In other embodiments, the NM-MRI technique determines the neuromelanin concentration in the locus coeruleus (LC) subregions. In further embodiments, the NM-MRI technique determines dopamine release in the dorsal striatum and resting blood flow within the locus coeruleus either directly or indirectly.

In some embodiments, the NM-MRI signal and Alzheimer's disease severity are directly correlated. In some embodiments, the NM-MRI signal and Alzheimer's disease severity are inversely correlated. In other embodiments, NM-MRI exhibits lower signal in the nigrostriatal pathway of people with Alzheimer's disease. In some embodiments, the NM-MRI captures dopamine dysfunction. In yet other embodiments, the NM-MRI can be used as a biomarker for Alzheimer's disease. In further embodiments, the NM-MRI can be used to determine the severity of Alzheimer's disease. In further embodiments, the NM-MRI can be used to diagnose and/or provide a prognosis for Alzheimer's disease.

In some embodiments, the analysis is performed in comparison to previous NM-MRI. In other embodiments, the analysis is performed in comparison to a reference value and/or range. In some embodiments, the reference value and/or range is generated using a compilation of neuromelanin data from healthy people. In some embodiments, the reference value and/or range is generated using a compilation of neuromelanin data from people who have Alzheimer's disease. In some embodiments, the reference value and/or range is generated using a compilation of neuromelanin data from people who have Alzheimer's disease and people who do not have Alzheimer's disease.

In some embodiments, the NM-MRI signal is taken from the substantia nigra or the locus coeruleus. In some embodiments, the NM-MRI signal is taken from both the SN and the locus coeruleus.

Certain embodiments of the present disclosure can provide an objective test to enhance diagnostic accuracy, advance the recognition of Alzheimer's disease into a presymptomatic stage, and serve as a monitor for therapy. In general, embodiments of the present disclosure can be used to diagnose neuromelanin using a stored template, differentiate between a number of different conditions or diseases, and monitor a subject over a period of time.

In one embodiment, the disclosure is used with a second imaging method, wherein the second imaging method is positron emission tomography (PET). In one embodiment, the disclosure is used with a second imaging method, wherein the second imaging method is structural MRI. In one embodiment, the disclosure is used with a second imaging method, wherein the second imaging method is functional MRI (fMRI). In one embodiment, the disclosure is used with a second imaging method, wherein the second imaging method is blood oxygen level dependent (BOLD) fMRI. In one embodiment, the disclosure is used with a second imaging method, wherein the second imaging method is iron sensitive MRI. In one embodiment, the disclosure is used with a second imaging method, wherein the second imaging method is quantitative susceptibility mapping (QSM). In one embodiment, the disclosure is used with a second imaging method, wherein the second imaging method is diffusion tensor imaging DTI. In one embodiment, the disclosure is used with a second imaging method, wherein the second imaging method is single photon emission computed tomography (SPECT). In one embodiment, the disclosure is used with a second imaging method, wherein the second imaging method is DaTscan. In one embodiment, the disclosure is used with a second imaging method, wherein the second imaging method is DaTquant.

In some embodiments, the neuromelanin concentration and/or level is measured against a control and if the neuromelanin concentration and/or level is about 5%, about 10%, about 15%, about 20%, about 25%, about 30%, about 35%, about 40%, about 50%, about 60%, about 70%, about 80%, about 90% less than the control a diagnosis of Alzheimer's disease is supported. In some embodiments, the change in neuromelanin is assessed as a net concentration or level change per year. In some embodiments, the change in neuromelanin is assessed as a percentage change per year. In some embodiments, the neuromelanin concentration and/or level is measured against a control and the neuromelanin concentration and/or level is about 1%, about 2%, about 3%, about 4%, about 5%, about 6%, about 7%, about 8%, about 9%, about 10%, about 11%, about 12%, about 13%, about 14%, or about 15% less than the control. In some embodiments, the neuromelanin concentration and/or level is measured against a control and the neuromelanin concentration and/or level is about 1%, about 2%, about 3%, about 4%, about 5%, about 6%, about 7%, about 8%, about 9%, about 10%, about 11%, about 12%, about 13%, about 14%, or about 15% decreased per year compared to the control.

In one embodiment, the control is a patient's prior NM-MRI scan. In one embodiment, neuromelanin concentration and/or level is measured against a control and the neuromelanin concentration and/or level is measured yearly, every 2 years, every 3 years, every 4 years, every 5 years, every 6 years, every 7 years, every 8 years, every 9 years, every 10 years, every 20 years. In one embodiment, the second time point is about 3 months, about 6 months, about 9 months, about 12 months, about 2 years, about 3 years, about 4 years, about 5 years, about 6 years, about 7 years, about 8 years, about 9 years, about 10 years, about 15 years, about 20 years, about 25 years, or about 30 years after the first time period. In certain embodiments, when the neuromelanin concentration and/or level is measured to be less than the control, a patient is diagnosed with Alzheimer's disease. In certain embodiments, when the neuromelanin concentration and/or level is measured to be a pre-determined amount less than the control either per year or net overall change, a patient is diagnosed with Alzheimer's disease. In further embodiments, the measured neuromelanin is more than about 20% less than the control. In further embodiments, the measured neuromelanin is more than about 25% less than the control. In further embodiments, the measured neuromelanin is more than about 30% less than the control. In further embodiments, the measured neuromelanin is more than about 35% less than the control. In further embodiments, the measured neuromelanin is more than about 45% less than the control. In further embodiments, the measured neuromelanin is more than about 40% less than the control. In further embodiments, the measured neuromelanin is more than about 50% less than the control. In certain embodiments, the control is optionally a previous neuromelanin MRI scan of the same patient. In other embodiments, the control comprises a reference number optionally determined from a database of neuromelanin MRI scans from at least one other person with the disease.

In one embodiment, if the change in the level, signal and/or concentration of neuromelanin at the second time point is more than about 5% less or more than about 10% less than the level, signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Alzheimer's disease is provided.

In one embodiment, if the change in the level, signal and/or concentration of neuromelanin at the second time point is more than about 35% less, more than about 40% less, more than about 45% less, or more than about 50% less signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Alzheimer's disease is provided.

In one embodiment, the degree of reduction in neuromelanin volume, signal, or concentration in a given patient compared to a control is proportional to the progression and/or severity of Alzheimer's disease.

In one embodiment, the degree of increase in neuromelanin volume, signal, or concentration in a given patient compared to a control is proportional to the improvement and/or efficacy of Alzheimer's disease progression and/or treatment.

In one embodiment, the standard control is a level of neuromelanin present at approximately the same levels in a population of subjects, or the standard control is approximately the average level of neuromelanin present in a population of subjects.

To illustrate extending the use of NM-MRI for such applications, a series of validation studies is shown. A first procedure is provided to show that NM-MRI can be sensitive enough to detect regional variability in tissue concentration of NM, which presumably depends on inter-individual and inter-regional differences in dopamine function (e g., including synthesis and storage capacity), and not just to loss of NM-containing neurons. To test this, MRI measurements were compared to neurochemical measurements of NM concentration in post-mortem tissue without Alzheimer's disease. Because variability in dopamine function may not occur uniformly throughout all SN tiers, the next procedure was to show that NM-MRI, which has high anatomical resolution compared to standard molecular-imaging procedures, has sufficient anatomical specificity. NM-MRI is used to test the ability of a novel voxelwise approach to capture the known topographical pattern of cell loss within the SN in Alzheimer's disease. The next procedure is then to provide direct evidence for a relationship between NM-MRI and Alzheimer's disease using the segmented approach.

As discussed in WO 2020/077098, incorporated herein in its entirety by reference, NM-MRI signal correlates to a well-validated Positron Emission Tomography (“PET”) measure of dopamine release into the striatum — the main projection site of SN neurons—and to a functional MRI measure of regional blood flow in the SN, an indirect measure of activity in SN neurons. Level of neuromelanin increases (SNc concentration, volume of NM in SNc), as measured the methods of the present disclosure, that results in improvement in UPDRS with L-Dopa therapy

In one embodiment, a representative treatment for any Alzheimer's disease is used. In one embodiment, the treatment is gene therapy. In one embodiment, if the neuromelanin concentration remains stable, unchanged or constant, the dosage of treatment remains constant. In one embodiment, if the neuromelanin concentration remains stable, the dosage of treatment is increased. In one embodiment, if the neuromelanin concentration is decreased by more than about 1%, more than about 2%, more than about 3%, more than about 5%, more than about 10%, more than about 15%, more than about 20%, or more than about 25%, then the Alzheimer's disease treatment dose is increased.

In one embodiment, the neuromelanin is monitored. In one embodiment, the set of controls from other patients is age matched. In one embodiment, the set of controls from other patients is gender matched.

In some embodiments, the neuromelanin is measured at least every other day, every week, every 2 weeks, every month, every other month, every 3 months, every 6 months, every year, every 2 years, every 3 years, every 4 years, every 5 years, every 6 years, every 7 years, every 8 years, every 9 years, every 10 years, every 15 years, every 20 years, every 25 years, every 30 years In certain embodiments, the second therapeutic agent dose is administered every week or every 2 weeks. In certain embodiments, the therapeutic is administered, every 1 hour, 2 hours, 3 hours, 4 hours, 5 hours, 6 hours, 8 hours, 10 hours, 12 hours, 14 hours, 16 hours, 18 hours, 20 hours, 24 hours, 1 day, 2 days, 3 days, 4 days, 5 days, 6 days, 7 days, or at least 14 days.

In one embodiment, the treatment period (either initial or subsequent) or monitoring period as discussed herein is every day, every other day, every 28 days, every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks, about every month, about every other month, about every 3 months, about every 6 months or about every year.

In some embodiments, the degree of reduction in neuromelanin volume, signal, or concentration in a given patient compared to a control is proportional to the progression and/or severity of Alzheimer's disease (AD) and neuropsychiatric symptoms (NPS).

In some embodiments, the degree of increase in neuromelanin volume, signal, or concentration in a given patient compared to a control is proportional to the improvement and/or efficacy of AD and/or NPS progression and/or treatment.

In some embodiments, the standard control is a level of neuromelanin present at approximately the same levels in a population of subjects, or the standard control is approximately the average level of neuromelanin present in a population of subjects.

The present disclosure correlates specific LC segments with AD and/or NPS symptoms as measured by Clinician-Administered AD and/or NPS Scale (CAPS); demonstrates that the application of the segmented-based analysis method finds specific LC segments (termed AD-segments and/or NPS-segments) either unique to each patient or consistent across a population of patients with the same disease that correlate with their specific symptoms on CAPS; determines the correlation between the change in neuromelanin measures after initiation of therapy and improvement in CAPS scores; determines the differences in neuromelanin measures (e.g. total NM concentration (microgram neuromelanin per microgram wet tissue) in locus coeruleus (LC), NM concentration in the subregions LC, volume of neuromelanin in the total LC, volume of subregions of the LC) in a patient with AD and/or NPS from the normal range of the control group; determines the difference in neuromelanin levels from a control group that would warrant a diagnosis of AD and/or NPS; correlates the change in neuromelanin measures after initiation of therapy and improvement in CAPS scores; determines the level of neuromelanin increase that results in improvement in CAPS to validate that NM levels can be used to monitor response to treatment; correlates AD and/or NPS segments with AD and/or NPS symptoms measured via CAPS scores; applies the segment based analysis method to find specific segments (termed AD and/or NPS segments) either unique to each patient or consistent across a population of patients with the same disease that correlate with their specific symptoms on CAPS; correlation between NM-MRI scans and both tau-PET or p-tau181 or ptau217 blood testing and CAPS scores.

In one embodiment, a region of interest is determined and segments that cover that region are measured to determine the volume of neuromelanin in that area.

In one embodiment, the region of interest is subdivided and segments that cover the subregions are measured to determine the volume of neuromelanin in that area.

In one embodiment, these segments are compared to a reference dataset and used to compute the concentration of neuromelanin in the region of interest or subregions within the region of interest.

In one embodiment, these segments are compared to a reference dataset and used to compute the total amount of neuromelanin in the region of interest or subregions within the region of interest.

In one embodiment, multiple comparisons are performed between all of the segments identified in the region of interest and specific symptoms or scales of symptom severity, or disease states, or demographic information, or other patient or disease-specific information, and associations are found between a subgroup of individual segments and a specific symptom or level of symptom severity on a disease monitoring scale. These are termed symptom-specific segments.

In one embodiment, multiple comparisons are performed between all of the segments identified in the region of interest and specific disease diagnoses or demographic information, or other patient or disease-specific information, and associations are found between a subgroup of individual segments and the condition of being diagnosed with a specific disease. These are termed disease-specific segments and in one example may comprise AD and/or NPS-disease-specific segments.

In one embodiment, these symptom-specific or disease-specific segments have similarities across multiple patients with the same symptom in the context of the same disease and can be used to make comparisons between multiple patients with the same disease (for example two patients with AD and/or NPS disease who both have the symptom of hyperarousal, sleep disturbances, or nightmares). In this case the similarities between patients may be compared and the symptom specific segments may function as a diagnostic biomarker for the symptom-specific, and disease-specific segments may function as a diagnostic biomarker for the specific disease.

In one embodiment, these symptom-specific or disease-specific segments have differences between patients with the same symptom occurring in the context of different diseases. In this case differences between the symptom specific segments can be used to differentiated between two different disorders sharing the same symptom.

In one embodiment, either symptom-specific segments or disease-specific segments, or neuromelanin concentrations, or neuromelanin volumes of specific regions or subregions can be used as a non-invasive biomarker to determine diagnostic information, to diagnose the presence of a specific disease (in this case AD and/or NPS disease or a related stress disorder such as acute stress disorder ASD).

In one embodiment, this can be accomplished by comparing the baseline measurements in a specific patient of either symptom-specific segments or disease-specific segments, or neuromelanin concentrations, or neuromelanin volumes of specific regions or subregions against future measurements of these values in the same patient.

In one embodiment, this can be accomplished by comparing the measurements in a specific patient of either symptom-specific segments or disease-specific segments, or neuromelanin concentrations, or neuromelanin volumes of specific regions or subregions against a standard control.

In one embodiment, either symptom-specific segments or disease-specific segments or neuromelanin concentrations, or neuromelanin volumes of specific regions or subregions can be used as a non-invasive biomarker to determine diagnostic information, to rule-out the presence of a related disorder or differentiate between related disorders such as AD and/or NPS and ASD.

In one embodiment, this can be accomplished by comparing the baseline measurements in a specific patient of either symptom-specific segments or disease-specific segments, or neuromelanin concentrations, or neuromelanin volumes of specific regions or subregions against future measurements of these values in the same patient.

In one embodiment, this can be accomplished by comparing the measurements in a specific patient of either symptom-specific segments or disease-specific segments, or neuromelanin concentrations, or neuromelanin volumes of specific regions or subregions against a standard control.

In one embodiment, either symptom-specific segments or disease-specific segments or neuromelanin concentrations, or neuromelanin volumes of specific regions or subregions can be used as a non-invasive biomarker to stage or grade a specific disease or symptom and differentiate or classify this information in a patient. For example, this may be used to determine the stage of AD and/or NPS or a stress disorder in a specific patient

In one embodiment, either symptom-specific segments or disease-specific segments or neuromelanin concentrations, or neuromelanin volumes of specific regions or subregions can be used as a non-invasive biomarker to determine the current severity of symptoms in a patient.

In one embodiment, either symptom-specific segments or disease-specific segments or neuromelanin concentrations, or neuromelanin volumes of specific regions or subregions can be used as a non-invasive biomarker to predict the development of new symptoms that the patient has not yet developed.

In one embodiment, either symptom-specific segments or disease-specific segments or neuromelanin concentrations, or neuromelanin volumes of specific regions or subregions can be used as a non-invasive biomarker to predict the severity of current symptoms, predict the future development of a disease course, or predict the response of either a specific symptom or the response of the disease as a whole response to treatment and function as a non-invasive prognostic biomarker.

In one embodiment, either symptom-specific segments or disease-specific segments or neuromelanin concentrations, or neuromelanin volumes of specific regions or subregions can be used as a non-invasive biomarker to monitor response to treatment for either a specific symptom or a disease state as a whole.

In one embodiment, either symptom-specific segments or disease-specific segments or neuromelanin concentrations, or neuromelanin volumes of specific regions or subregions can be used as a non-invasive biomarker to guide the selection of the correct treatment for either a specific symptom or a disease state as a whole.

In one embodiment, either symptom-specific segments or disease-specific segments or neuromelanin concentrations, or neuromelanin volumes of specific regions or subregions can be used as a non-invasive biomarker to determine the status of treatment and determine if an adequate response to treatment has been obtained for either a specific symptom or a disease state as a whole.

In one embodiment, either symptom-specific segments or disease-specific segments or neuromelanin concentrations, or neuromelanin volumes of specific regions or subregions can be used as a non-invasive biomarker to predict the future response to treatment for either a specific symptom or a disease state as a whole.

In any embodiment, comparisons may be made between:

The baseline measurements in a specific patient of either symptom-specific segments or disease-specific segments, or neuromelanin concentrations, or neuromelanin volumes of specific regions or subregions against future measurements of these values in the same patient.

The measurements in a specific patient of either symptom-specific segments or disease-specific segments, or neuromelanin concentrations, or neuromelanin volumes of specific regions or subregions against a standard control.

### Further PTSD and MDD Embodiments

In some embodiments, the degree of reduction in neuromelanin volume, signal, or concentration in a given patient compared to a control is proportional to the progression and/or severity of PTSD.

In some embodiments, the degree of increase in neuromelanin volume, signal, or concentration in a given patient compared to a control is proportional to the improvement and/or efficacy of PTSD and/or MDD progression and/or treatment.

In some embodiments, the standard control is a level of neuromelanin present at approximately the same levels in a population of subjects, or the standard control is approximately the average level of neuromelanin present in a population of subjects.

The present disclosure correlates specific LC segments with PTSD symptoms as measured by Clinician-Administered PTSD Scale (CAPS) and/or MDD using the DSM-5 diagnostic criteria or MINI criteria; demonstrates that the application of the segmented-based analysis method finds specific LC segments (termed either MDD-segments or PTSD-segments) either unique to each patient or consistent across a population of patients with the same disease that correlate with their specific symptoms on CAPS; determines the correlation between the change in neuromelanin measures after initiation of therapy and improvement in CAPS scores; determines the differences in neuromelanin measures (e.g. total NM concentration (microgram neuromelanin per microgram wet tissue) in locus coeruleus (LC), NM concentration in the subregions LC, volume of neuromelanin in the total LC, volume of subregions of the LC) in a patient with PTSD and/or MDD from the normal range of the control group; determines the difference in neuromelanin levels from a control group that would warrant a diagnosis of PTSD; correlates the change in neuromelanin measures after initiation of therapy and improvement in CAPS scores; determines the level of neuromelanin increase that results in improvement in CAPS to validate that NM levels can be used to monitor response to treatment; correlates PTSD and/or MDD segments with PTSD symptoms measured via CAPS scores and/or MDD measured via BDI-II total score or HAMD or MADRS scales; applies the segment based analysis method to find specific segments (termed PTSD and/or MDD segments) either unique to each patient or consistent across a population of patients with the same disease that correlate with their specific symptoms on CAPS.

In one embodiment, a region of interest is determined and segments that cover that region are measured to determine the volume of neuromelanin in that area.

In one embodiment, the region of interest is subdivided and segments that cover the subregions are measured to determine the volume of neuromelanin in that area.

In one embodiment, these segments are compared to a reference dataset and used to compute the concentration of neuromelanin in the region of interest or subregions within the region of interest.

In one embodiment, these segments are compared to a reference dataset and used to compute the total amount of neuromelanin in the region of interest or subregions within the region of interest.

In one embodiment, multiple comparisons are performed between all of the segments identified in the region of interest and specific symptoms or scales of symptom severity including CAPS, or disease states including PTSD and/or MDD, or demographic information, or other patient or disease-specific information, and associations are found between a subgroup of individual segments and a specific symptom or level of symptom severity on a disease monitoring scale. These are termed symptom-specific segments.

In one embodiment, multiple comparisons are performed between all of the segments identified in the region of interest and specific disease diagnoses or demographic information, or other patient or disease-specific information, and associations are found between a subgroup of individual segments and the condition of being diagnosed with a specific disease. These are termed disease-specific segments and in one example may comprise PTSD-disease-specific segments or MDD-disease-specific-segments.

In one embodiment, these symptom-specific or disease-specific segments have similarities across multiple patients with the same symptom in the context of the same disease and can be used to make comparisons between multiple patients with the same disease (for example two patients with PTSD who both have the symptom of hyperarousal, sleep disturbances, or nightmares, and/or MDD who have symptoms of anhedonia). In this case the similarities between patients may be compared and the symptom specific segments may function as a diagnostic biomarker for the symptom-specific, and disease-specific segments may function as a diagnostic biomarker for the specific disease.

In one embodiment, these symptom-specific or disease-specific segments have differences between patients with the same symptom occurring in the context of different diseases. In this case differences between the symptom specific segments can be used to differentiated between two different disorders sharing the same symptom.

In one embodiment, either symptom-specific segments or disease-specific segments, or neuromelanin concentrations, or neuromelanin volumes of specific regions or subregions can be used as a non-invasive biomarker to determine diagnostic information, to diagnose the presence of a specific disease (in this case PTSD or a related stress disorder such as acute stress disorder ASD, or panic disorder and/or MDD or a related depressive disorder including dysthymia, cyclothymia, bipolar disorder types I and II, adjustment disorder, or bereavement).

In one embodiment, this can be accomplished by comparing the baseline measurements in a specific patient of either symptom-specific segments or disease-specific segments, or neuromelanin concentrations, or neuromelanin volumes of specific regions or subregions against future measurements of these values in the same patient.

In one embodiment, this can be accomplished by comparing the measurements in a specific patient of either symptom-specific segments or disease-specific segments, or neuromelanin concentrations, or neuromelanin volumes of specific regions or subregions against a standard control.

In one embodiment, either symptom-specific segments or disease-specific segments or neuromelanin concentrations, or neuromelanin volumes of specific regions or subregions can be used as a non-invasive biomarker to determine diagnostic information, to rule-out the presence of a related disorder or differentiate between related disorders (in this case PTSD or a related stress disorder such as acute stress disorder ASD, or panic disorder and/or MDD or a related depressive disorder including dysthymia, cyclothymia, bipolar disorder types I and II, adjustment disorder, or bereavement).

In one embodiment, this can be accomplished by comparing the baseline measurements in a specific patient of either symptom-specific segments or disease-specific segments, or neuromelanin concentrations, or neuromelanin volumes of specific regions or subregions against future measurements of these values in the same patient.

In one embodiment, this can be accomplished by comparing the measurements in a specific patient of either symptom-specific segments or disease-specific segments, or neuromelanin concentrations, or neuromelanin volumes of specific regions or subregions against a standard control.

In one embodiment, either symptom-specific segments or disease-specific segments or neuromelanin concentrations, or neuromelanin volumes of specific regions or subregions can be used as a non-invasive biomarker to stage or grade a specific disease or symptom and differentiate or classify this information in a patient. For example, this may be used to determine the stage of PTSD, ASD, Panic disorder, or a related stress disorder in a specific patient

In one embodiment, either symptom-specific segments or disease-specific segments or neuromelanin concentrations, or neuromelanin volumes of specific regions or subregions can be used as a non-invasive biomarker to determine the current severity of symptoms in a patient including hyperarousal, sleep disturbances, and nightmares.

In one embodiment, either symptom-specific segments or disease-specific segments or neuromelanin concentrations, or neuromelanin volumes of specific regions or subregions can be used as a non-invasive biomarker to predict the development of new symptoms that the patient has not yet developed.

In one embodiment, either symptom-specific segments or disease-specific segments or neuromelanin concentrations, or neuromelanin volumes of specific regions or subregions can be used as a non-invasive biomarker to predict the severity of current symptoms, predict the future development of a disease course, or predict the response of either a specific symptom or the response of the disease as a whole response to treatment and function as a non-invasive prognostic biomarker. These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

In one embodiment, either symptom-specific segments or disease-specific segments or neuromelanin concentrations, or neuromelanin volumes of specific regions or subregions can be used as a non-invasive biomarker to monitor response to treatment for either a specific symptom or a disease state as a whole. These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

In one embodiment, either symptom-specific segments or disease-specific segments or neuromelanin concentrations, or neuromelanin volumes of specific regions or subregions can be used as a non-invasive biomarker to guide the selection of the correct treatment for either a specific symptom or a disease state as a whole. These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

In one embodiment, either symptom-specific segments or disease-specific segments or neuromelanin concentrations, or neuromelanin volumes of specific regions or subregions can be used as a non-invasive biomarker to determine the status of treatment and determine if an adequate response to treatment has been obtained for either a specific symptom or a disease state as a whole. These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

In one embodiment, either symptom-specific segments or disease-specific segments or neuromelanin concentrations, or neuromelanin volumes of specific regions or subregions can be used as a non-invasive biomarker to predict the future response to treatment for either a specific symptom or a disease state as a whole. These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

In any embodiment, comparisons may be made between:

The baseline measurements in a specific patient of either symptom-specific segments or disease-specific segments, or neuromelanin concentrations, or neuromelanin volumes of specific regions or subregions against future measurements of these values in the same patient.

the measurements in a specific patient of either symptom-specific segments or disease-specific segments, or neuromelanin concentrations, or neuromelanin volumes of specific regions or subregions against a standard control.

The measurements in a specific patient of either symptom-specific segments or disease-specific segments, or neuromelanin concentrations, or neuromelanin volumes of specific regions or subregions may be combined in our algorithm with information from a second imaging test including PET imaging, fMRI, and BOLD to obtain a more accurate diagnosis.

In some embodiments the level of NM in the SNc is measured with the voxel based algorithm and the level of NM in the LC is measured via the segmented based algorithm. These two readings together enable a more precise diagnosis than using either reading alone.

In some embodiments the concentration, volume, signal, and/or level of neuromelanin in the SNc is measured with a voxel based algorithm.

In some embodiments the concentration, volume, signal, and/or level of neuromelanin in the LC is measured via a segmented based algorithm.

In some embodiments, the concentration, volume, signal, and/or level of neuromelanin in the SNc is measured with the voxel based algorithm and the level of NM in the LC is measured via the segmented based algorithm.

In some embodiments, combining the two measurements enables a more precise diagnosis than using either measurement algorithm alone. In some embodiments, combining the two measurements enables distinguishing between diagnoses compared to using either measurement algorithm alone. In some embodiments, combining the two measurements enables distinguishing between similar diagnoses and selecting a more useful treatment regimen compared to using either measurement algorithm alone.

In some embodiments, the absolute difference between the degree of reduction in neuromelanin volume, signal, or concentration in a the SNc and the LC in a given patient compared to a control is proportional to the progression and/or severity of Parkinson's disease.

In some embodiments, the degree of increase in neuromelanin volume, signal, or concentration in the SNc and LC of a given patient compared to a control is proportional to the improvement and/or efficacy of Parkinson's disease progression and/or treatment.

In some embodiments, the standard control is a level of neuromelanin present at approximately the same levels in a population of subjects, or the standard control is approximately the average level of neuromelanin present in a population of subjects.

In some embodiments, the present disclosure correlates Parkinson's voxels with Parkinson's symptoms as measured by UPDRS; demonstrates that the application of the voxel-based analysis method locates specific voxels (termed PD voxels) unique to each patient that correlate with their specific symptoms on UPDRS; determines the correlation between the change in neuromelanin measures after initiation of L-DOPA therapy and improvement in UPDRS scores; determines the differences in neuromelanin measures (e.g. total NM concentration (microgram neuromelanin per microgram wet tissue) in substantia nigra pars compacta (SNc), NM concentration in the subregions SNc, volume of neuromelanin in the total SNc, volume of subregions of the SNc) in a patient with PD from the normal range of the control group; determines the difference in neuromelanin levels from a control group that would warrant a diagnosis of PD; correlates the change in neuromelanin measures after initiation of L-DOPA therapy and improvement in UPDRS scores; determines the level of neuromelanin increase that results in improvement in UPDRS to validate that NM levels can be used to monitor response to treatment; correlates Parkinson's voxels with Parkinson's symptoms measured via UPDRS scores; applies the voxel based analysis method to find specific voxels (termed PD voxels) unique to each patient that correlate with their specific symptoms on UPDRS; correlation between NM-MRI scans and both DaTscan and UPDRS scores.

In the voxels discussed herein, the diagnostic or prognostic value of specific voxels is enhanced when combined with data on NM segments in the LC obtained with the segmented based algorithm.

In one embodiment, a region of interest is determined and voxels that cover that region are measured to determine the volume of neuromelanin in that area.

In one embodiment, the region of interest is subdivided and voxels that cover the subregions are measured to determine the volume of neuromelanin in that area.

In one embodiment, these voxels are compared to a reference dataset and used to compute the concentration of neuromelanin in the region of interest or subregions within the region of interest.

In one embodiment, these voxels are compared to a reference dataset and used to compute the total amount of neuromelanin in the region of interest or subregions within the region of interest.

In one embodiment, multiple comparisons are performed between all of the voxels identified in the region of interest and specific symptoms or scales of symptom severity, or disease states, or demographic information, or other patient or disease-specific information, and associations are found between a subgroup of individual voxels and a specific symptom or level of symptom severity on a disease monitoring scale. These are termed symptom-specific voxels. In some embodiments, the ability is enhanced when combined with information on NM levels in segments in the LC as determined by the segmented based algorithm

In one embodiment, multiple comparisons are performed between all of the voxels identified in the region of interest and specific disease diagnoses or demographic information, or other patient or disease-specific information, and associations are found between a subgroup of individual voxels and the condition of being diagnosed with a specific disease. These are termed disease-specific voxels and in one example may comprise Parkinson's-disease-specific voxels. This ability is enhanced when combined with information on NM levels in segments in the LC as determined by the segmented based algorithm

In one embodiment, these symptom-specific or disease-specific voxels have similarities across multiple patients with the same symptom in the context of the same disease and can be used to make comparisons between multiple patients with the same disease (for example two patients with Parkinson's disease who both have the symptom of psychomotor slowing). In this case the similarities between patients may be compared and the symptom specific voxels may function as a diagnostic biomarker. This ability is enhanced when combined with information on NM levels in segments in the LC as determined by the segmented based algorithm

In one embodiment, these symptom-specific or disease-specific voxels have differences between patients with the same symptom occurring in the context of different diseases. In this case differences between the symptom specific voxels can be used to differentiated between two different disorders sharing the same symptom. This ability is enhanced when combined with information on NM levels in segments in the LC as determined by the segmented based algorithm

In one embodiment, either symptom-specific voxels or disease-specific voxels, or neuromelanin concentrations, or neuromelanin volumes of specific regions or subregions can be used as a non-invasive biomarker to determine diagnostic information, to diagnose the presence of a specific disease (in this case Parkinson's disease or a related disorder such as MSA, PSP, Parkinsonism symptoms, dyskinesia, dystonia, or essential tremor. This ability is enhanced when combined with information on NM levels in segments in the LC as determined by the segmented based algorithm

In one embodiment, this can be accomplished by comparing the baseline measurements in a specific patient of either symptom-specific voxels or disease-specific voxels, or neuromelanin concentrations, or neuromelanin volumes of specific regions or subregions against future measurements of these values in the same patient. This ability is enhanced when combined with information on NM levels in segments in the LC as determined by the segmented based algorithm

In one embodiment, this can be accomplished by comparing the measurements in a specific patient of either symptom-specific voxels or disease-specific voxels, or neuromelanin concentrations, or neuromelanin volumes of specific regions or subregions against a standard control. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to determine diagnostic information, to rule-out the presence of a related disorder or differentiate between related disorders such as Parkinson's disease and MSA, PSP, Parkinsonism symptoms, dyskinesia, dystonia, or essential tremor. This ability is enhanced when combined with information on NM levels in segments in the LC as determined by the segmented based algorithm

In one embodiment, this can be accomplished by comparing the baseline measurements in a specific patient of either symptom-specific voxels or disease-specific voxels, or neuromelanin concentrations, or neuromelanin volumes of specific regions or subregions against future measurements of these values in the same patient. This ability is enhanced when combined with information on NM levels in segments in the LC as determined by the segmented based algorithm

In one embodiment, this can be accomplished by comparing the measurements in a specific patient of either symptom-specific voxels or disease-specific voxels, or neuromelanin concentrations, or neuromelanin volumes of specific regions or subregions against a standard control. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to stage or grade a specific disease or symptom and differentiate or classify this information in a patient. For example, this may be used to determine the stage of PD or a related motor disorder in a specific patient This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to determine the current severity of symptoms in a patient. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to predict the development of new symptoms that the patient has not yet developed. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to predict the severity of current symptoms, predict the future development of a disease course, or predict the response of either a specific symptom or the response of the disease as a whole response to treatment and function as a non-invasive prognostic biomarker. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to monitor response to treatment for either a specific symptom or a disease state as a whole. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to guide the selection of the correct treatment for either a specific symptom or a disease state as a whole. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to determine the status of treatment and determine if an adequate response to treatment has been obtained for either a specific symptom or a disease state as a whole. This ability is enhanced when combined with information on NM levels in segments in the LC as determined by the segmented based algorithm

In one embodiment, either symptom-specific voxels or disease-specific voxels or neuromelanin concentrations, or neuromelanin volumes of specific regions or subregions can be used as a non-invasive biomarker to predict the future response to treatment for either a specific symptom or a disease state as a whole. This ability is enhanced when combined with information on NM levels in segments in the LC as determined by the segmented based algorithm

In any embodiment, comparisons may be made between:

The baseline measurements in a specific patient of either symptom-specific voxels or disease-specific voxels, or neuromelanin concentrations, or neuromelanin volumes of specific regions or subregions against future measurements of these values in the same patient. This ability is enhanced when combined with information on NM levels in segments in the LC as determined by the segmented based algorithm

the measurements in a specific patient of either symptom-specific voxels or disease-specific voxels, or neuromelanin concentrations, or neuromelanin volumes of specific regions or subregions against a standard control. This ability is enhanced when combined with information on NM levels in segments in the LC as determined by the segmented based algorithm.

In some embodiments, any method discussed herein with regard to a single neurological condition can be applied to any other neurological condition.

### LC and SNc Dual Analysis

In one embodiment, the prognosis and/or diagnosis of one or more neurological conditions can be determined using any of the methods discussed herein according to the following table:

| | SNc NM level | LC NM level determined |
|---|---|---|
| Neurological | determined with the | with the segmented based |
| Condition/Indication | voxel based algorithm | algorithm |
| Schizophrenia with | The higher the level | Normal/Unchanged |
| psychotic symptoms | the more severe the | |
| (prognostic) | symptoms | |
| Cocaine use disorder | High, about 14% | Trend to low |
| (diagnostic) | change; range about | |
| | 10% to about 20% | |
| Parkinson's disease | Low (about 23% | Normal/Unchanged |
| (diagnostic) | reduction) change | |
| | (reduction) above | |
| | about 10% | |
| Alzheimer's disease | Normal/Unchanged | Low less than (Range |
| without | | from about 10% to about |
| neuropsychiatric | | 15%l ower) |
| symptoms | | |
| (diagnostic) | | |
| Alzheimer's disease | The lower the level | The higher the level the |
| neuropsychiatric | the more severe the | more severe the symptoms |
| symptoms controlling | symptoms | |
| for the severity of the | | |
| illness (prognostic) | | |
| Major depressive | Normal/Unchanged | The lower the NM level |
| disorder (prognostic) | | the more severe the |
| | | symptoms |
| PTSD (diagnostic and | Normal/Unchanged | High (about 19% higher) |
| prognostic) | | Range greater than (about |
| | | 10%-about 25% increase) |
| | | Also the higher the NM |
| | | level the more severe the |
| | | symptoms |

In some embodiments, determining changes in the NM level, volume, or concentration of both the LC and SNc provide diagnostic or prognostic information, wherein the LC neuromelanin is determined by a segmented approach and the SNc neuromelanin is determined by a voxel wise approach.

In some embodiments, according to any of the methods described herein, the changes detected between NM-MRI scans or against a standard control are used to diagnose a neurological condition according to the Table above.

In some embodiments, according to any of the methods described herein, the changes detected between NM-MRI scans or against a standard control are used to provide a prognosis a neurological condition according to the Table above.

### Computer Based Analysis

Exemplary procedures in accordance with the disclosure described herein can be performed by a cloud-based processing arrangement and/or a computing arrangement (e.g., computer hardware arrangement). Such processing/computing arrangement can be, for example entirely or a part of, or include, but not limited to, a computer/processor that can include, for example one or more microprocessors, and use instructions stored on a computer-accessible medium (e.g., RAM, ROM, hard drive, or other storage device).

For example a computer-accessible medium (e.g., as described herein above, a storage device such as an encrypted cloud file, hard disk, floppy disk, memory stick, CD-ROM, RAM, ROM, etc., or a collection thereof) can be provided (e.g., in communication with the processing arrangement). The computer-accessible medium can contain executable instructions thereon. In addition or alternatively, a storage arrangement can be provided separately from the computer-accessible medium, which can provide the instructions to the processing arrangement so as to configure the processing arrangement to execute certain exemplary procedures, processes, and methods, as described herein above, for example.

Further, the exemplary processing arrangement can be provided with or include an input/output ports, which can include, for example a wired network, a wireless network, the internet, an intranet, a data collection probe, a sensor, etc. The exemplary processing arrangement can be in communication with an exemplary display arrangement, which, according to certain exemplary embodiments of the present disclosure, can be a touch-screen configured for inputting information to the processing arrangement in addition to outputting information from the processing arrangement, for example. Further, the exemplary display arrangement and/or a storage arrangement can be used to display and/or store data in a user-accessible format and/or user-readable format.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort can be had to various other embodiments, modifications, and equivalents thereof which can suggest themselves to those skilled in the art without departing from the spirit of the present disclosure and/or scope of the appended claims.

### Example 1: Correlation of Neuromelanin-Sensitive MRI Signal in the Locus Coeruleus to Cortical Tau Proliferation and Neuropsychiatric Symptoms

Neuropsychiatric symptoms (NPS) are a common and burdensome aspect of Alzheimer's disease (AD). Managing such symptoms, more so than cognitive deficits alone, often requires residential care. While some of these symptoms may not emerge until later illness stages, others can emerge at prodromal or even premorbid stages. Effective NPS treatment at the earliest stages could slow their progression and minimize associated complications. The most common existing treatments, antidepressants or antipsychotics, have variable efficacy perhaps due to the failure to target the neurobiological cause of the symptoms for a given patient.

AD pathophysiology and NPS: The physiological mechanism underlying these symptoms is poorly understood. They may be related to key pathophysiological changes occurring in AD including the accumulation of β-amyloid and phosphorylated tau. Tau and amyloid load in AD patients have been found to correlate to aggression, psychosis, and other NPS. The LC, the primary site of noradrenaline neurons, begins to degenerate early in AD and is the first brain region to accumulate hyperphosphorylated tau proteins at Braak stage 0. Compensatory changes occur in the noradrenergic system to restore balance, possibly even leading to hyperactivity in remaining LC neurons. The noradrenergic system is becoming a major target of interest in treatment of AD, especially regarding NPS. Noradrenergic disturbances correlate to NPS in AD and may have a causal role because symptoms of agitation/aggression and depression respond to treatment with noradrenergic drugs. Regarding the direction of the relationship, depressive symptoms in AD have been linked to LC degeneration and low noradrenergic function, while total, aggressive, and psychotic symptoms in AD have been linked to high or preserved noradrenergic function. Our study examining these characteristic pathophysiological AD features will be strongly positioned to extend existing model of how these insults interact to promote NPS at early illness stages.

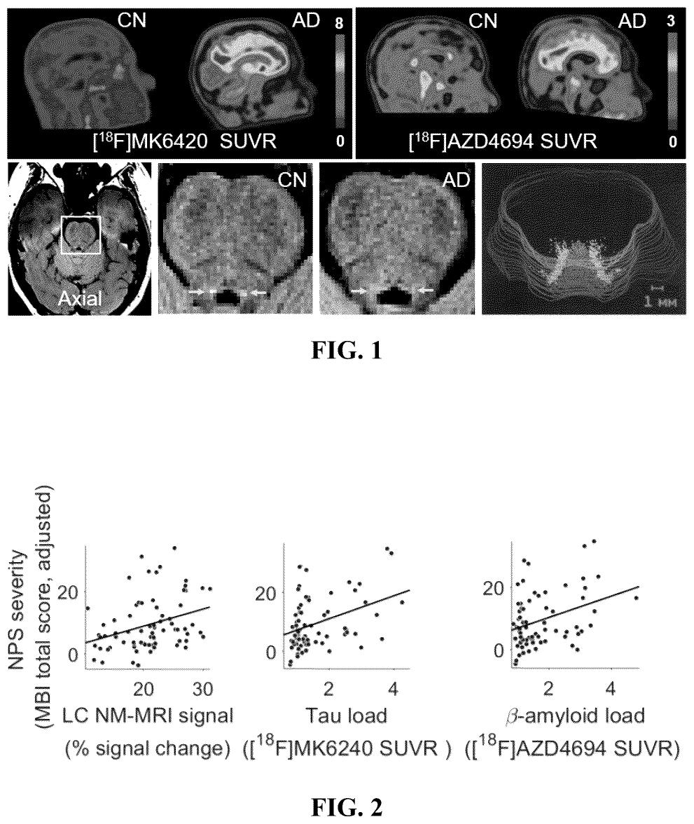

Advanced neuroimaging measures of AD pathophysiology: Advances in MRI and PET imaging allow measurement of LC degeneration and β-amyloid and tau load in anatomical detail within living human brain. Our group has extensive experience with all these tools. More than 300 PET scans have been successfully collected using the tracers [18F]AZD4694 (for amyloid) and [18F]MK6240 (for tau; see FIG. 1). These validated tracers allow in vivo AD diagnosis and Braak Staging. Our group has validated NM-MRI as a measure of the structure and function of catecholamine neurons. As seen in FIG. 1, NM-MRI captures degeneration of the noradrenergic system in AD as a loss of signal (secondary to loss of neuromelanin pigment) in the LC. A caveat in LC NM-MRI is the small size of this structure (cross-sectional diameter of 1-2 mm, FIG. 1) which is at the limit of what can be detected using high field (3 Tesla) MRI. Recent advances have developed ultra-high field (7T) NM-MRI sequences that increase image resolution (5.5× in our case) and thereby reduce measurement noise (FIG. 3 ). While no studies have yet tested the advantages of 7T NM-MRI in AD, at 3T this method not only reveals LC degeneration in AD but in other populations it correlates to symptoms resembling NPS including depression, sleep disturbance, and autonomic regulation.

Combining these advanced neuroimaging methods with assessment of NPS using a highly sensitive instrument designed for use prior to dementia onset (MBI) will allow us to model mechanisms through which AD-associated brain changes lead to the emergence of NPS across illness stages.

### Participants and Clinical Measures

Study participants from the community or outpatients at the McGill University Research Centre for Studies in Aging were enrolled in the Translational Biomarkers of Aging and Dementia (TRIAD) cohort, McGill University, Canada. The cohort participants had a detailed clinical assessment, including the Clinical Dementia Rating (CDR) and Mini-Mental State Examination (MMSE). Cognitively unimpaired participants had no objective cognitive impairment and a CDR score of 0. Mild cognitive impairment (MCI) individuals had subjective and objective cognitive impairments, preserved activities of daily living, and a CDR score of 0.5. Patients with mild-to-moderate sporadic Alzheimer's disease dementia had a CDR score between 0.5 and 2, and met the National Institute on Aging and the Alzheimer's Association criteria for probable Alzheimer's disease determined by a physician (McKhann et al., 2011). Sporadic early-onset Alzheimer's disease dementia were individuals with dementia onset before 65 years (Snowden et al., 2011). Participants were excluded if they had inadequately treated conditions, active substance abuse, recent head trauma, or major surgery, or if they had MRI/PET safety contraindication. Alzheimer's disease patients did not discontinue medications for this study.

NPS severity was assessed using the Mild Behavioral Impairment Checklist (MBI-C, http://www.MBItest.org). The MBI-C was completed by the participant's primary informant, most frequently their spouse. The MBI-C is composed of 34 questions and subdivided into five domains: (1) decreased drive and motivation (apathy), (2) affective dysregulation (mood and anxiety symptoms), (3) impulse dyscontrol (agitation, impulsivity, and abnormal reward salience), (4) social inappropriateness (impaired social cognition), and (5) abnormal perception and thought content (psychotic symptoms). Each question is answered with “Yes” or “No,” and a severity rating is accorded to each question answered “Yes” of either 1=mild, 2=moderate, or 3=severe. To be given a “Yes” rating, symptoms must have persisted for at least 6 months. This study was approved by the Douglas Mental Health University Institute Research Ethics Board and Montreal Neurological Instituted PET working committee, and written informed consent was obtained from all participants.

TABLE 1
Clinical and demographic measures

| | | | | P Value | | |
|---|---|---|---|---|---|---|
| | CN | MCI | AD | CN vs. | CN vs. | MCI vs. |
| Characteristic | (n = 112) | (n = 51) | (n = 28) | AD | MCI | AD |
| Age, mean | 72.0 (5.9) | 72.5 (7.2) | 67.1 (8.9) | <.001 | .63 | .004 |
| (SD), y | | | | | | |
| Male, No. (%) | 36 (32.1) | 22 (43.1) | 12 (42.9) | .29 | .17 | .98 |
| Education, | 15.6 (3.6) | 14.3 (3.5) | 15.0 (3.7) | .45 | .03 | .38 |
| mean (SD), y | | | | | | |
| CDR score, | 0.1 (0.2) | 0.2 (0.3) | 0.5 (0.6) | <.001 | .009 | .01 |
| mean (SD) | | | | | | |
| MMSE score, | 29.3 (0.9) | 28.0 (1.8) | 20.9 (5.9) | <.001 | <.001 | <.001 |
| mean (SD) | | | | | | |
| MBI total | 2.4 (5.4) | 7.5 (8.4) | 13.0 (9.8) | <.001 | <.001 | .01 |
| score, mean | | | | | | |
| (SD) | | | | | | |
| Beta-amyloid | 28 (25.5) | 31 (60.8) | 28 (100.0) | <.001 | <.001 | <.001 |
| positive, No. | | | | | | |
| (%) | | | | | | |
| Tau-positive, | 24 (21.8) | 19 (37.3) | 26 (92.9) | <.001 | .04 | <.001 |
| No. (%) | | | | | | |

### MRI Acquisition

All neuroimaging data were acquired at the Montreal Neurological Institute. Magnetic resonance (MR) images were acquired on a 3T Prisma scanner. NM-MRI images were collected via a turbo spin echo (TSE) sequence with the following parameters: repetition time (TR)=600 ms; echo time (TE)=10 ms; flip angle=120°; in-plane resolution=0.7×0.7 mm²; partial brain coverage with field of view (FoV)=165×220; number of slices=20; slice thickness=1.8 mm; number of averages=7; acquisition time=8.45 min. The slice-prescription protocol consisted of orienting the image stack along the anterior-commissure-posterior-commissure line and placing the top slice 3 mm above the floor of the third ventricle, viewed on a sagittal plane in the middle of the brain. Whole-brain, high-resolution T1-weighted MRI images were also acquired for preprocessing of the NM-MRI and PET data using an MPRAGE sequence (inversion time=1050 ms, TR=2500, TE=1.69 ms, flip angle=7°, FoV=192×192, matrix=193×256, number of slices=256 slices, isotropic voxel size=1 mm, acquisition time=5.47 min). Quality of MRI images was visually inspected for artifacts immediately upon acquisition, and scans were repeated when necessary, time permitting.

### Preprocessing of NM-MRI Images

Initial preprocessing steps were performed as in our prior work examining NM-MRI signal from the substantia nigra (5) using SPM12. Although final analyses of LC signal were performed on native-space NM-MRI images, it was necessary, nonetheless, to spatially normalize the NM-MRI images to register a universal LC search space from MNI space to native space for each participant. NM-MRI scans were first coregistered to participants' T1-weighted scans. Tissue segmentation was then performed using the T1-weighted images. NM-MRI scans were normalized to MNI space using DARTEL routines with a gray- and white-matter template generated from all study participants. The resampled voxel size of these normalized NM-MRI scans was 1 mm, isotropic. All images were visually inspected after each of these steps. A visualization template was created by averaging the spatially normalized NM-MRI images from all participants.

Subsequent steps were developed using custom Matlab scripts to specifically examine the LC signal. An over-inclusive LC mask was drawn over the visualization template to cover the hyperintense voxels at the anterior-lateral edge of the 4^{th} ventricle spanning 15 mm in the rostrocaudal axis (from MNI space coordinates z=−16 to −31, see FIG. 1). The rostrocaudal limits were set by cross referencing distance to anatomical landmarks (inferior collicus on rostral end and posterior recess of 4 th ventricle on caudal end) in brain atlases. A divided version of the mask was made by division into 5 rostrocaudal segments of equal length. The over-inclusive whole LC mask and the divided masks were then warped to native space using the inverse of the flow fields generated in the spatial normalization step and resampled to NM-MRI image space. The warped over-inclusive whole LC mask could then be used to define a search space within which to find the LC for each participant. A cluster-forming algorithm was used to segment the LC within this space, defined as the 4 adjacent voxels (1.96 mm²) with the highest mean signal. This operation was repeated for the right and left LC. Contrast-to-noise ratio (CNR) for each voxel v in a given axial slice was calculated as the relative difference in NM-MRI signal intensity I from a reference region RR in the same slice as: CNRᵥ=(Iᵥ−mode(I_{RR}))/mode(I_{RR}). A reference was used from region known to have low NM concentration, the central pons, defined by a circle of radius 11.6 mm, and centered 32.6 mm from the axis connecting right and left LC. Every axial slice in native space was identified as belonging to one of 5 rostrocaudal LC segments based on which of the 5 divided LC masks was present on the slice (if 2 of these masks were present on the same slice, the LC segment was defined by the mask covering the most LC voxels). LC signal was calculated for each of the five segments by averaging NM-MRI CNR values from the brightest voxel on every side and every slice that was determined to fall within that segment. The brightest voxel per slice was selected rather than the average of all LC voxels to minimize partial volume effects.

### PET Acquisition and Analysis

All individuals had ¹⁸F-AZD4694 and ¹⁸F-MK-6240 PET scans acquired with a brain-dedicated Siemens High Resolution Research Tomograph. See previous studies for more detailed PET methods. Tau tangle ¹⁸F-MK-6240 images were acquired at 90-110 min after the intravenous bolus injection of the tracer and were reconstructed using an OSEM algorithm on a 4D volume with four frames (4 300 s) (Pascoal et al., 2018b). Amyloid-b ¹⁸F-AZD4694 images were acquired at 40-70 min after the intravenous bolus injection of the tracer, and scans were reconstructed with the same OSEM algorithm on a 4D volume with three frames (3 600 s) (Cselenyi et al., 2012). At the end of each PET acquisition, a 6-min transmission scan was conducted with a rotating 137Cs point source for attenuation correction. The images were additionally corrected for motion, dead time, decay, and random and scattered coincidences. Briefly, T1-weighted MRIs were non-uniformity and field distortions corrected. PET images were then automatically registered to T1-weighted image space, and the T1-weighted images were linearly and non-linearly registered to the MNI reference space (Mazziotta et al., 1995). PET images were meninges and skull stripped and non-linearly registered to the MNI space using the transformations from the T1-weighted image to MNI space and from the PET image to T1-weighted image space. ¹⁸F-MK-6240 standardized uptake value ratio (SUVR) and ¹⁸F-AZD4694 SUVR used the inferior cerebellum and whole cerebellum grey matter as the reference region, respectively (Cselenyi et al., 2012; Pascoal et al., 2018b). PET images were spatially smoothed to achieve a final 8-mm full-width at half-maximum resolution Global ¹⁸F-AZD4694 SUVR values were estimated for the whole cortex (Pascoal et al., 2018a). ¹⁸F-MK-6240 SUVR values were estimated for Braak stage regions proposed by Braak (Braak and Braak, 1991, 1997; Braak et al., 2006; Braak et al., 2011; Braak I (transentorhinal), Braak II (entorhinal and hippocampus), Braak III (amygdala, parahippocampal gyms, fusiform gyms, lingual gyrus), Braak IV (insula, inferior temporal, lateral temporal, posterior cingulate, and inferior parietal), Braak V (orbitofrontal, superior temporal, inferior frontal, cuneus, anterior cingulate, supramarginal gyrus, lateral occipital, precuneus, superior parietal, superior frontal, rostro medial frontal), and Braak VI (paracentral, postcentral, precentral, and pericalcarine)). Subjects were divided into 3 groups based on Braak stage: tau negative (¹⁸F-MK-6240 SUVR in Braak region 1<1.2), Braak stage 1 positive (¹⁸F-MK-6240 SUVR in Braak stage 1 regions>1.2; these cases would be at Braak stage 1 or 2), and Braak stage 3 positive (¹⁸F-MK-6240 SUVR in Braak stage 3 regions>1.5; these cases would be at Braak stage 3 or higher). There were no discordant cases where tau was above threshold in stage 3 regions but not stage 1 regions.

### Statistical Analysis

Statistical tests relating final imaging measures to each other and to clinical measures were performed on Matlab software. These included ANCOVAs with Tukey's post-hoc tests, linear regression analyses, and partial Spearman correlations. See results for details of the specific models used. Voxel-wise statistics on ¹⁸F-MK-6240 SUVR maps were performed using Matlab software version 9.2 (http://www.mathworks.com) with VoxelStats package (Mathotaarachchi et al., 2016).

### Results

### LC NM-MRI Signal and Braak Stage

First, the fact LC NM-MRI signal was reduced in AD was confirmed. The LC NM-MRI signal was examined, averaged throughout the whole LC, in 191 older individuals who were divided based on cognitive status (with 2 levels: cognitively normal and cognitively impaired (AD and MCI individuals)) and tau-status (with 3 levels: tau negative, tau positive in Braak region 1 (threshold SUV=1.2 in region 1 ROI), and tau positive in Braak stage 3 region (threshold SUV=1.5 in region 3 ROI; see FIG. 3 for definition of ROIs)). A 2-way ANOVA on whole LC NM-MRI signal controlling for age and sex showed a significant effect of tau status (F_{2,184}=4.53, p=0.012) and no significant effect of cognitive status (F_{1,184}=0.34, p=0.56; model controlling for age and sex; a more complex model showed no significant tau status X cognitive status interaction, p=0.48). Post-hoc testing found a significant difference between tau negative individuals and Braak 3 positive individuals (p=0.012) but no difference between Braak 1 (only) positive individuals and either tau negative (p=0.17) or Braak 3 positive individuals (p=0.27, Tukey's HSD).

Next, the structure of tau-status-related signal loss was examined within subdivisions of the LC. Aside from the rostral and caudal ends of the LC, all segments significantly differed between tau groups, and the middle segments showed the strongest effect (FIG. 1). Examining the middle LC segment (average of the NM-MRI signal from this segment on left and right sides) a very strong relationship to tau status (F_{2,184}=15.3 p<0.00001) was found with a reduction in signal in Braak 3 positive individuals relative to both tau negative individuals (p<0.00001) and Braak 1 positive individuals (p=0.0045) but no reduction in signal in Braak 1 positive individuals relative to tau negative individuals (p=0.075, Tukey's HSD; FIG. 1. shows LC middle-segment signal for all study groups). Given these strong effects present in the middle LC segment, this was retained as the LC NM-MRI measure to be used for all subsequent analyses.

These results suggested that LC signal loss is minimal at Braak stage 1 and pronounced from Braak stage 3 onwards. To determine if there was evidence of progressive loss of LC signal from Braak stage 3, the cognitive impairment and dementia stage was correlated to LC signal in Braak 3 positive individuals. Both measures were significantly correlated to loss of LC signal (MMSE errors: t₂₃=−2.95, p=0.0072, robust linear regression; Clinical Dementia Rating Scale: Spearman ρ=−0.52, p=0.012, n=25; partial correlation; both analyses controlled for age, and sex; see FIG. 1).

### LC NM-MRI Signal and AD Pathophysiology

To more fully investigate the relationship of LC NM-MRI signal to tau proliferation, a voxelwise analysis relating LC signal to [¹⁸F]MK-6240 uptake was performed throughout the brain. Significant clusters were found in regions (see FIG. 6). To investigate how direct the link may be between LC signal and tau load, we performed a subsequent voxelwise analysis also controlling for cortical beta amyloid load and cortical gray matter volume.

To examine which aspects of AD pathophysiology independently predicted loss of LC NM-MRI signal, a linear regression analysis was performed in the cognitively impaired individuals (n=75); due to very high collinearity (r=0.91), summary measures of tau and amyloid were not included together in the same model. LC NM-MRI signal was significantly predicted by tau load in Braak region 3 (t₇₀=−2.36, p=0.021, linear regression controlling for CDR score, age, and sex) and by cortical amyloid load (t₇₀=−2.50, p=0.015, linear regression with the same covariates). However, when including cortical gray matter volume into either of these models it became a significant predictor of LC NM-MRI signal (for the tau model: t₆₈=2.13, p=0.037, linear regression controlling for tau load in Braak region 3, cortical gray matter volume, total intracranial volume, CDR score, age, and sex)and neither tau nor amyloid remained as significant predictors.

LC NM-MRI Signal and Clinical Presentation

Finally, the clinical correlates of LC NM-MRI signal loss was investigated. Specifically, both cognitive impairment and neuropsychiatric symptoms relating each to LC signal was examined while controlling for key pathophysiological measures. In cognitively impaired individuals (n=72), LC signal did not significantly correlate to the extent of cognitive impairment (MMSE score: t₆₄=0.83; linear regression controlling for tau load in Braak region 3, cortical gray matter volume, total intracranial volume, CDR score, age, and sex).

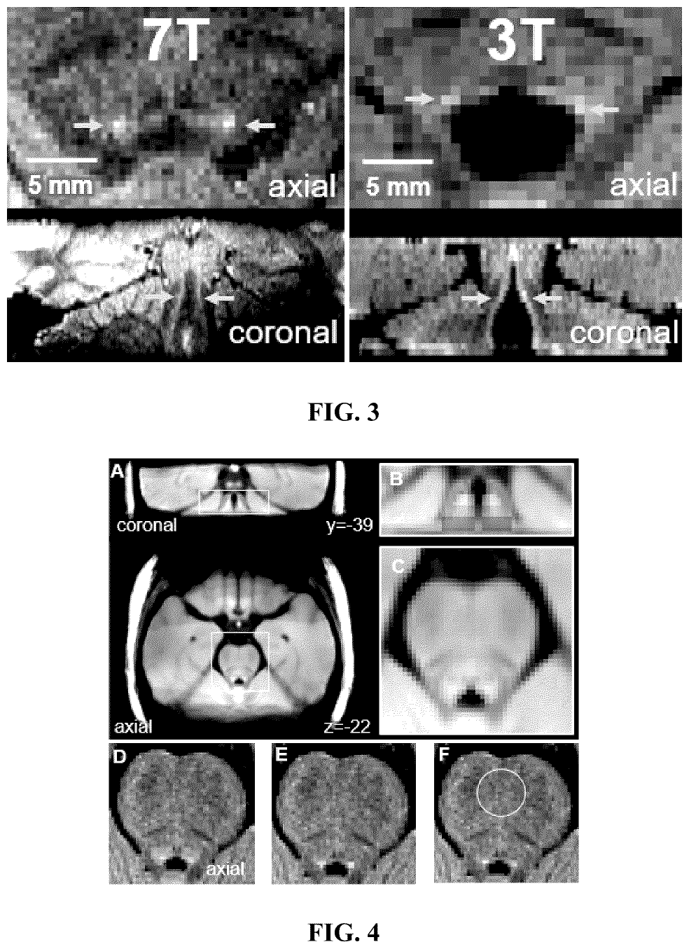

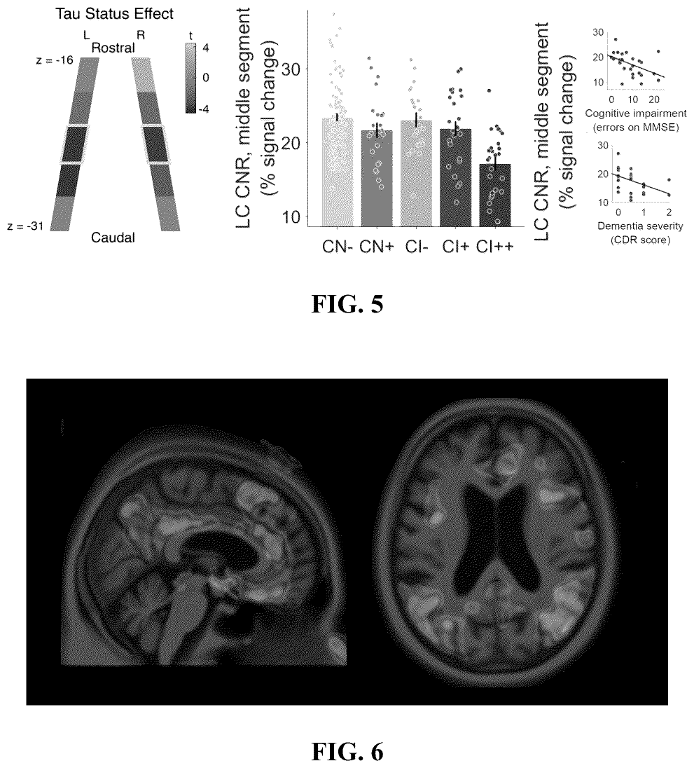

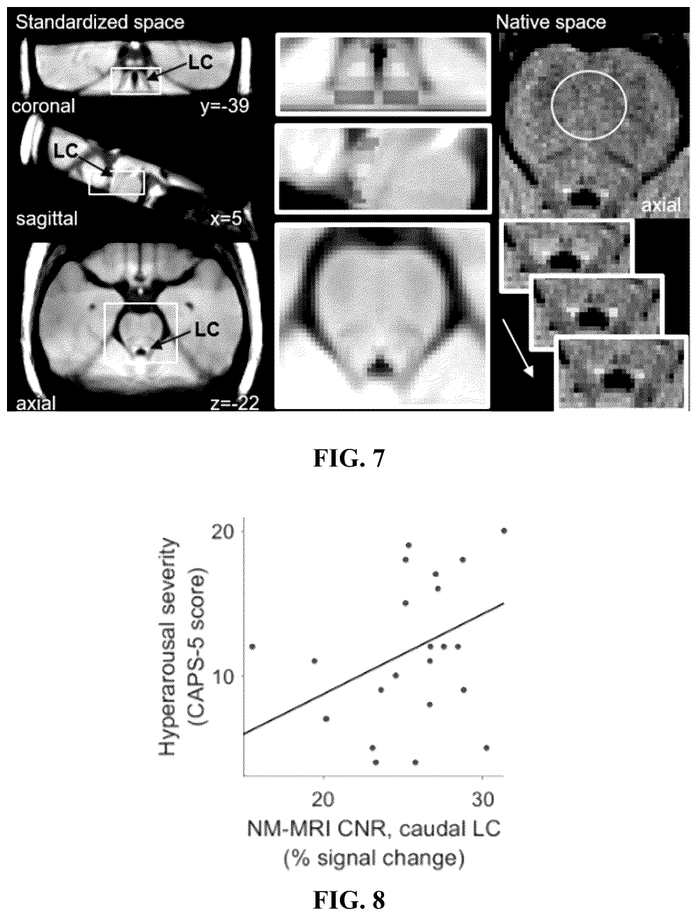

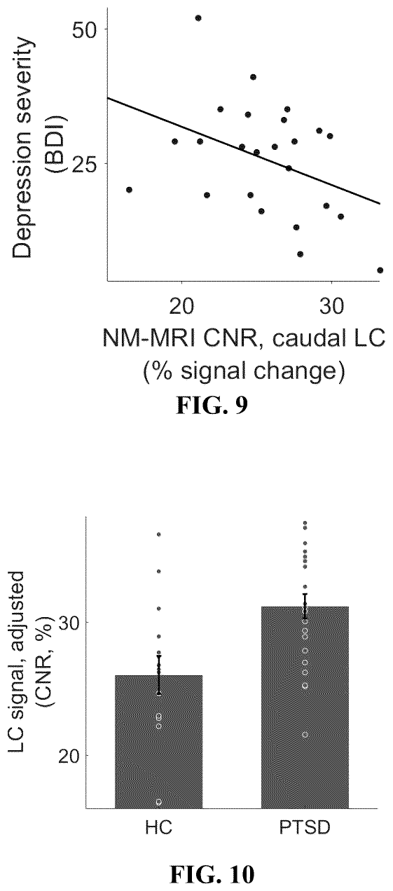

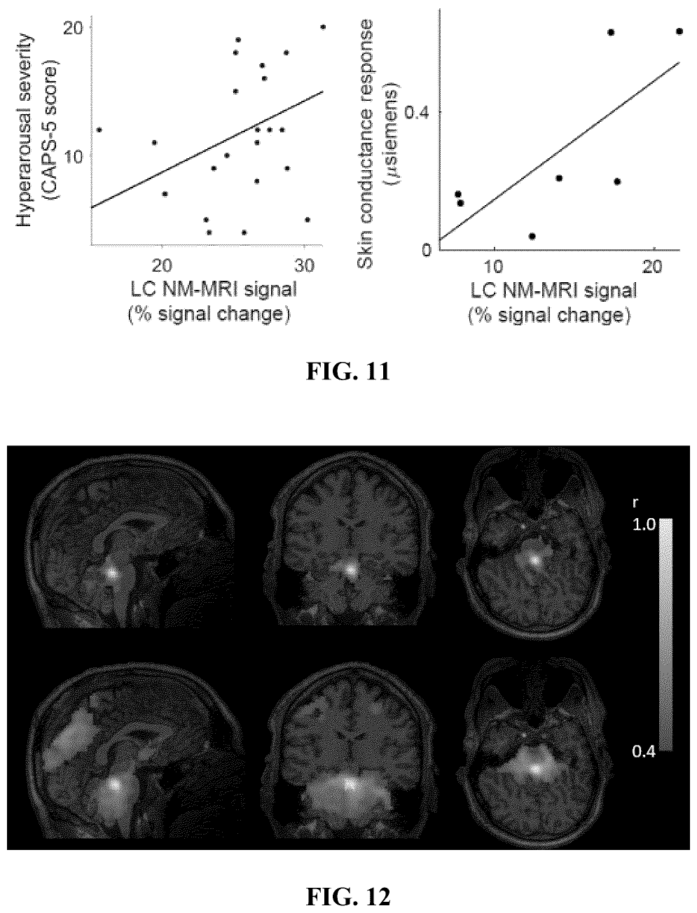

Next, neuropsychiatric symptoms were considered as measured with the total score on the Mild Behavioral Impairment Checklist (MBI). This measure was significantly related to LC NM-MRI signal in cognitively impaired individuals (n=73) irrespective of which pathophysiological measures were included as covariates (Table xx). In a preferred model, LC NM-MRI signal and tau load in Braak region 3 both predicted MBI total score (t₆₅=3.48, p=0.0009 and t₆₅=2.48, p=0.016, respectively; linear regression also controlling for cortical gray matter volume, total intracranial volume, CDR score, age, and sex; FIG. 4). The correlation of LC NM-MRI signal to MBI total score was confirmed in non-parametric testing with the same covariates as the linear regression (Table xx, for the preferred model Spearman ρ=0.40). This positive relationship suggests that preservation of the LC is associated with worse NPS and it is notable that this effect becomes stronger when any measure of cortical pathology is included in the model. Post-hoc analyses examined the MBI subdomains and found the domain where the relationship to the LC NM-MRI signal was strongest was Impulse Dyscontrol (Spearman ρ=0.36, p=0.003, partial correlation controlling for tau load, cortical gray matter volume, total intracranial volume, CDR score, age, and sex).

When pedicting MBI total score in cognitively normal older adults, LC NM-MRI signal was not a significant predictor (t₉₂=−0.19, p=0.85) nor was tau load in Braak region 1 (t₉₂=1.91, p=0.059; linear regression controlling for cortical gray matter volume, total intracranial volume, CDR score, age, and sex).

TABLE 2
Prediction of neuropsychiatric symptom severity (MBI total score) in
cognitively impaired individuals

| | | t-statistic for regression coefficient | | | | Spearman |
|---|---|---|---|---|---|---|
| | | to predict MBI total score | | | | partial |
| | | | | | Cortical | correlation, p |
| predictors | | LC NM- | Tau in | | gray | LC signal |
| in model; | Adj | MRI | Braak | Cortical | matter | and MBI |
| df | R2 | signal | region 3 | amyloid | volume | total score |
| 1; 4.68 | 0.06 | 2.01* | — | — | — | 0.24* |
| 2; 5.67 | 0.24 | 3.27** | 4.06*** | — | — | 0.38** |
| 2; 5.67 | 0.20 | 3.18** | — | 3.54*** | — | 0.36** |
| 2; 6.66 | 0.17 | 3.23** | — | — | −3.30** | 0.37** |
| 3; 7.65 | 0.24 | 3.48*** | 2.48* | — | −0.96 | 0.40*** |

Analyses included age, gender, and CDR score as covariates (analyses including cortical gray matter volume also included total intracranial volume as a covariate).
*p < 0.05,
**p < 0.01,
***p < 0.001

### Example 2: A Longitudinal Multimodal Neuroimaging Study to Determine LC, Amyloid, and Tau Signatures of Upcoming Progression of Neuropsychiatric Symptoms in Patients with MCI and AD as Well as CN Older Adults

LC NM-MRI signal, together with amyloid and tau load in key brain regions, predict progression of NPS, 18 months later. This is examined separately in cognitively impaired (AD and MCI) and unimpaired older adults to determine predictions even at the earliest illness stages.

Data obtained demonstrate that NPS are linked to tau accumulation, β-amyloid accumulation and LC integrity as measured in vivo with PET [18F]MK6240, [18F]AZD4694 and neuromelanin-sensitive MRI (NM-MRI), respectively.

Without being bound to any theory, NPS reflects an imbalance in the key pathophysiological changes occurring in AD: integrity of the LC on one hand and amyloid and tau accumulation in the cortex on the other hand. The combined effects of these processes may lead to an imbalance in cortical and subcortical regulation of behavior, triggering emergence of NPS. Our preliminary data reveal a pattern that is associated with NPS in the early course of illness: cortical tau accumulation, combined with preservation of the LC, perhaps reflecting dysregulation of cortical control of an intact or even hyperactive LC and leading to expression of NPS including impulse dyscontrol and emotional dysregulation. This is also consistent with reports of elevated noradrenergic activity and tau pathology (measured in CSF) both correlating to worse NPS and noradrenergic blockade treating NPS. A linear regression model is being used to predict NPS including all neuroimaging measures, specifically that LC NM-MRI signal, tau load, and amyloid load will all be positively related to progression/emergence of NPS, and the combined prediction in models including all measures will be superior to prediction using any one measure alone. Identifying which neuropathological process is most implicated in a given patient is a critical step before targeted NPS treatment can become a reality.

NPS severity (MBI total score, n=73) from LC NM-MRI signal, tau load (SUVR of [¹⁸F]MK6240 in Braak stage 3 ROI), and β-amyloid load (SUVR of [¹⁸F]AZD4694 in cortical ROI). Multivariate prediction is superior to univariate for all measures.

| | | t stat for LC | |
|---|---|---|---|
| Predictors in linear | Adjusted | signal regression | t stat for tau/amyloid |
| regression model | R² | coefficient | regression coefficient |
| LC NM-MRI | 0.06 | 2.02* | — |
| Tau load | 0.13 | — | 3.08** |
| Amyloid load | 0.09 | — | 2.52* |
| LC and tau load | 0.24 | 3.29** | 4.09*** |
| LC and amyloid load | 0.20 | 3.21** | 3.57*** |
| LC, tau, and amyloid | 0.23 | 3.22** | 1.81, −0.01 |

All analyses controlled for age, gender, and dementia severity (Clinical Dementia Rating Scale score).
*p < 0.05,
**p < 0.01,
***p < 0.001

The heterogeneity of NPS is an important consideration. In our sample, MBI total score is most highly correlated to the Impulse Dyscontrol subdomain (r=0.85), a symptom type with a strong theoretical link to the noradrenergic system. Depressive symptoms, on the other hand, relate to low noradrenergic function (reduced LC NM-MRI signal); thus, analyses will consider depressive symptoms separately

### Study Design and Timeline

The study consists of baseline clinical and neuropsychological assessments, amyloid and tau PET scans, and MRI scans in n=70 CN older adults, n=35 with MCI, and n=35 with AD. Participants return 18 months later for another clinical and neuropsychological assessment. Participants will be already involved in a longitudinal protocol, the Translational Biomarkers in Aging and dementia (TRIAD), facilitating follow-up contact. Timeline: months 1-6 ethical approvals, staff training, and optimization of the 7T NM-MRI sequence. Months 7-30, recruitment of participants. To achieve our sample of n=140 (after attrition) we recruited 77 individuals per year. Similar to the TRIAD cohort where 100-130 participants complete MRI and PET imaging procedures annually. Quality control and preprocessing of neuroimaging data is ongoing as data is collected; preliminary analysis is done when half of the sample is collected. Months 24-48, follow-up clinical and cognitive assessments.

### Recruitment and Consent

All participants are recruited from a longitudinal PET biomarker cohort: the Translational Biomarkers in Aging and dementia (TRIAD) at the McGill Centre for Studies in Aging (MCSA). The MCSA has a clinical database of 4000 patients from which the TRIAD cohort is primarily drawn which has already recruited over 1000 people willing to participate in studies.

### Inclusion Criteria

Participants who are 55 to 90 years old and have a history of academic achievement to exclude intellectual disability are enrolled. Participants are excluded if they are unable to provide informed consent, or if they become unable to provide consent during the study. Their ability to provide consent will be determined using the screening battery, which includes MMSE and MoCA. Cognitively normal older individuals (CN) are defined by a clinical dementia rating (CDR) of 0. MCI are defined by a CDR of 0.5, subjective and objective memory loss, and having normal activities of daily living. CN and MCI groups have absence of dementia, based on the Petersen and National Institute of Ageing—Alzheimer' s Association Criteria. AD cases will be of mild severity, defined as a CDR of 0.5-1.0 and diagnosed using the National Institute of Ageing—Alzheimer's Association criteria.

### Exclusion Criteria

(i) Illiterate (not due to cognitive decline), (ii) use of recreational drugs, (iii) major structural abnormality or major vascular pathology on MRI exam, (iv) participated in intervention trial within prior 4 weeks or were exposed to ionizing radiation (via research study or radiological exam) within prior 12 months, (v) contra-indications to MRI/PET scans, (vi) chronic and recurrent mental health conditions; i.e. a past history of psychiatric disorder (e.g. schizophrenia, major depression, PTSD). Those with new onset psychiatric symptomatology will be included, unless the severity precludes participation (e.g. violence or aggression).

### Neuropsychiatric Symptoms and Other Clinical and Cognitive Measures

Measures of NPS provide comprehensive assessment of a wide range of types of NPS and are sensitive to detect their expression at different illness phases. These include standard questionnaires used in AD: the Neuropsychiatric Inventory (NPI), the Apathy Inventory, and the Epworth sleep questionnaire. In addition, a specialized questionnaire is employed designed to be sensitive for assessment of NPS in prodromal AD, the Mild Behavioral Impairment Checklist (developed by Dr. Ismail). Standard protocols used in the TRIAD cohort are followed for assessment of clinical profiles and cognition. Cognitive measures include the Rey Auditory Verbal Learning Test (RAVLT), digit span and digit symbol from the WAIS-III, and IQ (WASI-II; matrix reasoning, vocabulary; part of a 3-hour battery administered by a neuropsychologist). These measures are recorded every 24 months in the TRIAD protocol and will not be repeated for participants tested within 60 days of baseline or follow-up assessment. Our primary measure of interest is change in the MBI over 18 months. Preliminary follow-up data show that this measure is able to capture change over the course of 1 or 2 years.

### NM-MRI Acquisition at 7T

Participants are scanned on a Siemens Terra 7T scanner with an 8-channel transmit and 32-channel receive head coil (Nova Medical). A magnetization transfer-prepared turbo-flash sequence (MTw-TFL), developed by the lab of Christine Tardif, is used to image the LC. The MT preparation consists of a train of 15 pulses of 1 ms duration (2 ms gap) at an offset frequency of 10 kHz, with a B1 root-mean-squared value of 9 μT. The polarity of the frequency offset alternates between pulses. Each MT preparation block is followed by a center-out TFL readout (TE/TR=4.3/505 ms; turbo factor=29; flip angle=8°; GRAPPA=2), scan time=4:31 mins at a resolution of 0.4×0.4×1.0 mm3. The sequence is repeated twice and averaged to increase SNR. The further optimization of this sequence to maximize SNR and reliability of the LC signal to improve on this state-of-the art sequence is occurring. A high-resolution (0.65 mm isotropic) T1-weighted anatomical scan using the MP2RAGE sequence: TI1/T12=1000/32000 ms, TR=43000 ms, TE=2.46, alpha=4°, echo spacing=7.5 ms, slice partial Fourier 6/8, GRAPPA=3, scan time=11 mins is performed. For subjects willing to tolerate a longer session, resting-state blood oxygen level dependent (BOLD) functional MRI data is also collected for exploratory analysis of functional connectivity changes correlated to NPS. For this purpose, a 2D gradient echo EPI sequence at 1.8 mm isotropic: TE=25 ms, TR=2010 ms, alpha=70°, GRAPPA=2, phase partial Fourier= 6/8, and scan time=10.5 mins is sued. Total scanning time will be ˜40 minutes if all scans are completed.

### NM-MRI Preprocessing and Analysis

The LC NM-MRI signal is measured on unprocessed NM-MRI images in native space using a custom script and SPM12 tools to segment the LC, similar to approaches used previously. Analyzing the signal from this small structure in native space is advantageous compared to the conventional approach of working in standardized space for MRI analysis. NM-MRI preprocessing pipeline developed by Dr. Cassidy.

### PET Acquisition

PET scanning takes place on a Siemens HRRT. Radiotracers are produced by the Centre's radiochemistry laboratory and cyclotron. A PET scan and the MRI scan are be conducted on the same day. In a subsequent day, another PET scan is conducted. [18F]AZD4694 or [18F]MK6240 PET scans are acquired following administration of 185 MBq of the tracer. The scan using [18F]MK6240 is 20 mins long, beginning 90-110 minutes post-injection. The scan using [18F]AZD4694 is 30 mins, beginning 40-70 minutes post-injection. Subjects wear specialized glasses for correction of head movement. Dynamic images are acquired using list mode file. Transmission images are acquired with a Ge-68 source. Tissue radioactivity images will be re-binned using 4 frames and reconstructed using a OSM3 method, with scatter and attenuation correction. Movement correction is then be applied.

### PET Analysis

Quantification of [18F]AZD4694 or [18F]MK6240 PET will be performed for both anatomical regions of interest (ROI) and individual voxel maps. In both instances, as a first step, the MRI volume (T1-weighted image) will be segmented to obtain gray matter maps and then co-registered to the PET images through rigid transformation using MINC tools. Respective [18F]AZD4694 SUVR50-70 or [18F]MK6240 SUVR90-110 will be analyzed using the cerebellum cortex as a reference region. MRI scans will be transformed into standard MNI space using non-linear registration. In the case of ROI analysis, the inverse transformation of the registration parameters will be used to map a probabilistic anatomical atlas back onto the PET image. Regional time-activity curves (TACs) will be calculated through a mask obtained by the convolution of the segmented gray-matter image of the subject and the atlas. Both Region of Interest (ROI) and voxel TAC will then be entered in the appropriate quantification procedure to obtain SUVRs for the set of anatomical ROI or BP parametric maps for the voxel-by-voxel analysis. Partial volume corrections (PVC) will be conducted to all images. Voxel-based Analysis will be performed by first warping the parametric maps into MNI space using the non-linear registration procedure described above. Parametric maps will then be smoothed (6 mm) to reduce noise. Voxel level univariate testing using the general linear model (GLM) will then be applied with post-hoc multiple comparison correction derived from random-fields theory as implemented in voxel-stat, a suite to perform voxel-based generalized linear models developed at McGill University.

Statistical analyses: The primary analyses for hypothesis testing are linear regressions to predict change in NPS severity 18 months after baseline, based on baseline LC NM-MRI signal, amyloid load, and tau load in ROIs where these substances appear in early disease stages (Braak stage 3 regions for tau and the entire cerebral cortex for amyloid). The effects of interest are the independent effect of LC NM-MRI signal and the increase in explained variance with the additional inclusion of amyloid and/or tau load into the model. Analysis in CN cases use total score on the MBI as the outcome measure (a highly sensitive instrument suitable for cases with minimal symptom burden). In CI individuals (MCI and AD), analyses predict total score on both the MBI and the NPI. Post-hoc testing includes voxelwise analyses using the same predictors and outcomes (within masks of areas implicated in early tau/amyloid accumulation to minimize the penalty for multiple comparisons) and also ROI analyses using more specific types of NPS as the outcome measure (e.g. impulse dyscontrol, sleep problems, aggressive behavior). For all analyses, possible interactions between LC NM-MRI signal and amyloid/tau in the prediction of NPS severity are examined. Analyses include covariates age, sex, dementia severity (Clinical Dementia Rating Scale), and severity of depression (NPI depression item). Controlling for depression severity is necessary because noradrenergic function may have an opposing relationship to depression as compared to symptoms such as aggression and impulsivity (the latter correlating very highly to our primary measure of interest, MBI total score). Conversely, analyses predicting depressive symptoms (NPI depression) control for severity of other NPS.

Sex and gender-based analysis: Sex effects are observed in late-life depression; for instance, regarding its link to cognitive impairment, functional impact, and brain structure. Furthermore, animal studies report sex dimorphism on the relationship between LC damage and tau phosphorylation, consistent with a female predisposition for norepinephrine-related disorders. Thus, sex may be a factor in these relationships. An equal number of males and females (previous recruitment from the TRIAD cohort was 63% female) is recruited. Primary analysis models are run separately in men and women and determine if the strength of the effects significantly differs by sex.

The results have a lasting impact for dementia patients and those at risk. Around 75% of AD patients and 50% of individuals with mild cognitive impairment (MCI) suffer from NPS compared to 25% of individuals showing normal cognitive aging. The presence of these symptoms is associated with faster cognitive and functional decline, lower quality of life, earlier admission to a nursing home, and greater caregiver burden. As existing treatments for neuropsychiatric symptoms in AD have limited efficacy for many patients with a substantial risk of harm, it is imperative to understand their neurobiology to find and monitor improved treatments. The biomarkers described herein help guide development of new NPS treatment and optimization of existing treatments ultimately supporting precision medicine approaches to identify patients most likely to respond to certain NPS treatments. One NPS treatment target is the noradrenaline system, the integrity of which is measured via the LC NM-MRI signal described herein. Indeed, since NM-MRI is a practical and non-invasive assay of neurochemical changes underlying AD pathology, this could prove to be a useful tool, for instance as a potential moderator of NPS treatment response or as a marker of response to LC neuroprotective drugs. There is promising evidence for both these applications: aggressive behavior in AD patients is more likely to respond to noradrenergic drug treatment in patients exhibiting noradrenergic dysfunction and to guide an LC neuroprotective drug that lowers NPS-like behaviors in animal models of AD.

### Example 3: NM MRI for Assessing Post Traumatic Stress Disorder (PTSD) and Major Depressive Disorder

Introduction

Post-traumatic stress disorder (PTSD), is a heterogenous condition that diminishes the quality of life of military veterans and confers an important risk of suicide. Given the complex expression of the illness, treatment targeting specific neurobiological disruptions in a patient-specific manner may be needed. However, the search for biomarkers to support targeted treatment in PTSD has been challenging. Recent research has suggested that dysregulation of the neuromodulator norepinephrine (NE) may contribute to PTSD symptomatology. The locus coeruleus (LC) is the central nucleus for NE release in the human brain and the LC-NE system plays an important role with respect to regulation of the stress response, autonomic function, emotional memory, sleep, and arousal. These LC-regulated behaviors are particularly relevant to the hyperarousal symptom domain of PTSD, defined by the DSM-5 as exaggerated startle response, hypervigilance, and sleep disturbances. For example, individuals with PTSD have been observed to show higher LC BOLD fMRI activation to stimulus in comparison to control. It has also been shown that there is a relationship between autonomic system dysregulation and the severity of hyperarousal symptoms. For example, in a study by Blechert et al., in 2007, individuals with PTSD showed “attenuated parasympathetic and elevated sympathetic control, as evidenced by low respiratory sinus arrhythmia (a measure of cardiac vagal control) and high electrodermal activity”. Given the evidence of the role of the NE system in PTSD, efforts have increased to deploy pharmacological treatments targeting this system. Several drugs acting on this system have shown benefits, including venlafaxine, a mixed NE/serotonin reuptake inhibitor that is a common PTSD treatment and has been shown to outperform specific serotonin reuptake inhibitors, and beta-blockers, when used in conjunction with trauma re-experiencing psychotherapy. Furthermore, the NE alpha-1 receptor antagonist prazosin, has shown inconsistent evidence of efficacy in PTSD, underscoring the potential advantages of a biomarker tracking noradrenergic imbalance that could pre-select likely responders to a noradrenergic drug like prazosin, and thereby also support trials of experimental noradrenergic treatments.

Neuromelanin-sensitive magnetic resonance imaging (NM-MRI) is a novel and non-invasive neuroimaging method that can be used to image the NM-containing structures in the human brain: the LC and the dopaminergic substantia nigra due to the paramagnetic properties of NM. We have previously shown that the NM signal in the substantia nigra, can provide a proxy measure for PET imaging metrics of dopamine function with the practical advantages of being less expensive, non-invasive, and obtainable at high resolution. We propose here that the LC NM signal can provide similar insight into function of the NE system. Although the LC NM-MRI signal has yet to be investigated in PTSD, there is evidence it tracks measures of NE or autonomic function: it has been correlated to heart rate variability, alpha amylase secretion, and anxious-arousal symptoms in anxiety disorder. We are particularly interested in the caudal LC here since this region sends descending projections to the autonomic nervous system, has enhanced activity in PTSD and correlates to autonomic measures.

Here we examined the relationship of the LC NM-MRI signal to hyperarousal symptoms, a dimensional measure of PTSD psychopathology, in a sample of 24 help-seeking Canadian Armed Forces (CAF) veterans with a history of operational deployment. We hypothesized that NM-MRI signal in the caudal LC will positively correlate to hyperarousal symptom severity measured with the Clinician Administered PTSD scale for DSM-5 (CAPS-5).

### Methods

### Participants

Twenty-three CAF veterans with a history of operational deployment were recruited from the Operational Stress Injury Clinic at The Royal Mental Health Center in Ottawa, Ontario. 18 of these individuals met DSM-5 criteria for PTSD as determined by the Clinician Administered PTSD scale for DSM-5 (CAPS-5). CAPS interviews were carried out by trained raters. See Table 1 for all clinical and demographic measures. Severity of depressive symptoms was assessed with the Beck Depression Inventory-II (21 item version). Other clinical assessments included, the Life Events Checklist for the DSM-5, the Pittsburgh Sleep Quality Index, and the Columbia Suicide Severity Rating Scale. Inclusion criteria included: aged between 18 and 65 years, CAF veteran with a history of operational deployment after the year 2000. Exclusion criteria included history of manic/hypomanic or psychotic disorder, diagnosis of a substance use disorder (SUD) in the last 6 month, having a major medical illness, neurological condition, traumatic brain injury (or head trauma with a loss of consciousness of at least 5 minutes), the inability to abstain from alcohol, nicotine, cannabis or caffeine for a 24 hour period, and current use of stimulant medication (due to possible impact on the NM-MRI signal). This study was approved by the ethics review board at The Royal Mental Health Centre in Ottawa, Ontario and participants provided written informed consent.

### MRI Acquisition

Magnetic resonance (MR) images were acquired for all study participants on a Siemens 3T PET BIOGRAPH mMR scanner using a 12-channel head coil. NM-MRI images were collected via a 2D gradient response echo sequence with magnetization transfer contrast (2D GRE- MT) with the following parameters: repetition time (TR)=337 ms; echo time (TE)=3.97 ms; flip angle=50°; in-plane resolution=0.43×0.43 mm²; partial brain coverage with field of view (FoV)=165×220; matrix=384×512; number of slices=10; slice thickness=3 mm; slice gap=0 mm; magnetization transfer frequency offset=1200 Hz; number of excitations (NEX)=6; acquisition time=7.24 min. The slice-prescription protocol consisted of orienting the image stack along the anterior-commissure-posterior-commissure line and placing the top slice 3 mm above the floor of the third ventricle, viewed on a sagittal plane in the middle of the brain.

Whole-brain, high-resolution T1-weighted MRI images were also acquired for preprocessing of the NM-MRI data using an MEMPRAGE sequence (inversion time =1050 ms, TR=2500, TE=1.69 ms, flip angle=7°, FoV=192×192, matrix=192×256, number of slices=256 slices, isotropic voxel size=1 mm, acquisition time=5.47 min. Quality of NM-MRI images was visually inspected for artifacts immediately upon acquisition, and scans were repeated when necessary, time permitting.

### Preprocessing of NM-MRI Images

Initial preprocessing steps were performed as in our prior work examining NM-MRI signal from the substantia nigra (5) using SPM12. Although final analyses of LC signal were performed on native-space NM-MRI images, it was necessary, nonetheless, to spatially normalize the NM-MRI images to register a universal LC search space from MNI space to native space for each participant. NM-MRI scans were first coregistered to participants' T1-weighted scans. Tissue segmentation was then performed using the T1-weighted images. NM-MRI scans were normalized to MNI space using DARTEL routines with a gray- and white-matter template generated from all study participants. The resampled voxel size of these normalized NM-MRI scans was 1 mm, isotropic. All images were visually inspected after each of these steps. A visualization template was created by averaging the spatially normalized NM-MRI images from all participants.

Subsequent steps were developed using custom Matlab scripts to specifically examine the LC signal. An over-inclusive LC mask was drawn over the visualization template to cover the hyperintense voxels at the anterior-lateral edge of the 4^{th} ventricle spanning from z=xx-xy in the rostrocaudal axis (see FIG. 1). The rostrocaudal limits were set by cross referencing distance to anatomical landmarks (inferior collicus on rostral end and posterior recess of 4^{th} ventricle on caudal end) in brain atlases. A divided version of the mask was made by division into 3 rostrocaudal segments of equal length. The over-inclusive whole LC mask and the divided masks were then warped to native space using the inverse of the flow fields generated in the spatial normalization step and resampled to NM-MRI image space. The warped over-inclusive whole LC mask could then be used to define a search space within which to find the LC for each participant. A cluster-forming algorithm was used to segment the LC within this space, defined as the 6 adjacent voxels (2.58 mm²) with the highest mean signal. This operation was repeated for the right and left LC. Contrast-to-noise ratio (CNR) for each voxel v in a given axial slice was calculated as the relative difference in NM-MRI signal intensity I from a reference region RR in the same slice as: CNRᵥ=(Iᵥ−mode(I_{RR}))/mode(I_{RR}). We used a reference region known to have low NM concentration, the central pons, defined by a circle of radius xx mm, and centered x mm from the axis connecting right and left LC. Every axial slice in native space was identified as belonging to LC segment 1 (rostral), 2 (middle), or 3 (caudal) based on which of the 3 divided LC masks was present on the slice (if 2 of these masks were present on the same slice, the LC segment was defined to match the mask covering brighter LC voxels). LC signal was calculated for each of the three segments by averaging NM-MRI CNR values from all voxels determined to fall within that segment (e.g. if a segment covered 2 axial slices in native space on both the right and left sides, this would be the mean of CNR from 24 voxels—6 voxels per LC*2 sides*2 slices).

### Statistical Analysis

Final statistical analyses were calculated using Matlab. Partial correlations examined the relationship of LC NM-MRI signal to clinical measures including covariates age, sex, and PTSD diagnosis. Key measures (LC NM-MRI signal, hyperarousal severity, and depression severity) were found to be normally distributed based on Lilliefors test, supporting our use of parametric statistics.

### Results

This sample of veterans with a history of operational deployment showed relatively high levels of hyperarousal and depressive symptoms (present both in those with and without PTSD diagnosis, see Table 3). As hypothesized, NM-MRI signal in the caudal LC was significantly positively correlated with severity of CAPS-5 hyperarousal symptoms (r=0.52, p=0.019, partial correlation controlling for depression severity, PTSD diagnosis, age, and sex; see FIG. 2). Consistent with studies in other populations, we observed a significant, negative correlation between caudal LC NM signal and depression severity (BDI-II total severity score, r=−0.48, p=0.033,partial correlation controlling for hyperarousal severity, age, sex, and PTSD diagnosis. Finally, although our sample of participants not meeting PTSD criteria was very small (n=5), we tested whether there was evidence of an effect of PTSD diagnosis on the NM-MRI signal (FIG. 4). We found a trend level effect that trended towards significance in this analysis (t₂₀=−1.0, p=0.32, linear regression controlling for hyperarousal severity, depression severity, age, and sex). This final analysis was severely underpowered and we hypothesize our trend level effect will reach significance in a larger future study.

Together, the above results demonstrate altered LC activation in psychiatric conditions such as PTSD and depression. Specifically, the significant, positive correlation between LC NM-MRI signal and hyperarousal symptomology in individuals with PTSD. Our current research also links increased LC-NE activity and its correlation to hyperarousal symptoms

Furthermore, in studies looking at pharmacological interventions for PTSD, numerous adrenergic drugs have been shown to be somewhat effective at treating symptoms associated with the hyperarousal symptom cluster. Specifically, there is moderate evidence to support the use of prazosin, an alpha-1 adrenergic receptor antagonist to treat nightmares amongst individuals with PTSD. Particularly, it has shown to be of aid to veterans who suffer hyperarousal symptoms associated with their PTSD. Consequently however, prazosin has also been shown to be ineffective in other individuals PTSD, further demonstrating the complexities associated with this condition. For example, in a meta-analysis conducted amongst military members, clinical studies involving numerous medications were analyzed, specifically focusing on the efficacy of each medication. Here, amongst 106 trials only 6% of individuals had a full, successful response to prazosin. 51% demonstrated no response at all to the drug. It is well known that PTSD is a heterogeneous condition and as such more research is required to further identify biomarkers associated with this illness.

In regard to, the negative correlation between LC NM-MRI signal and depression severity, altered LC-NE system activity has also been hypothesized for major depression and our results support the use of NM-MRI as a biomarker for MDD. In pharmacological studies focusing on targets of innervation for major depression, the locus coeruleus' NE receptors have been investigated. Here, when selective NE reuptake inhibitor, reboxetine was administered to individuals with depression, the drug s=had a very similar efficacy as tricyclic antidepressants. Other drugs that target both the serotine system and norepinephrine system (serotonin and norepinephrine reuptake inhibitors), have also showed to be effective at treating symptoms associated with depression. Furthermore, in a post-mortem study conducted amongst individuals with depression, marked alterations in NE neuron density and decreased NE transporter binding have been observed in the locus coeruleus in individuals with depression. All in all, our results align with the current literature suggesting reduced NE and thus altered LC-NE activity in individuals with depression.

With respect to our methods used in this study, we previously demonstrated the method's utility and validity in capturing alterations in the dopamine system. In this study we were also able to further validate our semi-automated methodology for extracting the LC NM-MRI signal in the LC. Here, our method provides a unique approach to NM image analysis of the LC. With this value and our observed success in capturing and analyzing NM data, we are confident in our methods and their ability to capture changes in the LC-NE system, specifically in a clinical setting

Furthermore, by utilizing a novel neuroimaging method within the field of psychiatry, past challenges faced by researchers studying both the dopamine and norepinephrine neurotransmitter systems, can be overcome using this method. Specifically, using this method we are able to achieve increase in-plane resolution of the LC and as such are better able to capture changes related to LC activity. NM-MRI is also a novel imaging technique that has not been previously used in PTSD research, thus adding a unique aspect to our current research.

Limitations of our current study include the small sample size, lack of a defined, healthy control group which resulted in our finding a trend toward significance in LC NM-MRI signal and overall PTSD diagnosis. To address these limitations in the future, increased recruitment for both our PTSD group as well as a defined control group should be conducted. The lack of a large healthy control group can be difficult to come by in PTSD-related research since many individuals who have experienced a traumatic event, but did not go on to develop PTSD have other underlying mental health concerns, hence why we compared our PTSD group to individuals who did not have PTSD but did have depression. Furthermore, with respect to the correlation between LC NM-MRI signal and PTSD diagnosis, an increased sample size may should this correlation more evident and we hypothesize our trend toward significance observed here will reach significance if true healthy controls are incorporated.

To conclude, the results of the present study indicate that NM is a biomarker for PTSD and depression and support the use of our segmented based algorithm to measure NM in patients with these diseases. The correlations between LC NM-MRI signal and PTSD and depression provide clinical evidence to support altered NE activity and as such provides further evidence to support the role of the NE system in both conditions. This research may also provide insight into future noradrenergic targets for the treatment of both conditions.

TABLE 3
Participant demographical and clinical data

| | PTSD | Controls (No PTSD) |
|---|---|---|
| Sample size (n=) | 18 | 5 |
| Age (average, in years) | 46.72 | 51.2 |
| Sex (males: females) | 11:7 (39% female) | 5:0 (0% female) |
| PTSD Diagnosis (n=)_ | 18 (100%) | 0 (0%) |
| MDD Diagnosis (n=) | 7 (39%) | 2 (40%) |
| (according to MINI) | | |
| Antidepressant use (n=) | 8 (44%) | 3 (60%) |
| Cannabis Use (n=) | 5 (28%) | 2 (40%) |
| BDI-II (average total | 48.65 | 46 |
| severity score) | | |
| CAPS-5 (average total | 40.11 | 28.2 |
| severity score) | | |
| CAPS-5 (average | 12.2 | 9 |
| hyperarousal severity | | |
| score) | | |
| Multiscale Dissociation | 56.53 | 56.4 |
| Inventory (average total | | |
| severity score) | | |

Example 4. NM as a Biomarker for PTSD Using the Segmented Based Approach

### Summary

The current study proposes neuromelanin-sensitive MRI (NM-MRD as a novel biomarker allowing directed treatment attacking the hyperarousal symptom cluster in PTSD. Such symptoms can lead to significant functional impairment and suicidality and no specific, neuroscience-based treatment currently exists. NM- MRI, a brief and non-invasive MRI scan, could provide a practical and reliable marker of norepinephrine (NE) excess in PTSD and thereby allow neurobiologically-informed treatment decisions. We propose to test this approach by linking hyperarousal symptoms to the NM-MRI signal in the current proposal and by using NM-MRI to predict treatment response in subsequent clinical trials.

Neuromelanin is a pigment that lends the bluish color to NE neurons in the locus coeruleus (LC). It is formed from the metabolism of NE and slowly accumulates over the lifespan. Validation work from our group has established that, unlike most neurochemicals, NM content can be measured at high resolution with a specialized MRI sequence, NM-MRI. This method is practical for widespread clinical use: it is non- invasive and runs on any MRI scanner in under 10 minutes. Work by our group and others suggests that NM-MRI may provide a neurochemical foundation for a key PTSD endophenotype, exaggerated sympathetic and hyperarousal responses. Thus this technique may provide a stable measure of NE imbalance in PTSD, yet the method remains untested in PTSD and is very innovative and novel.

Excess NE function may be a key component of PTSD; however, this may be the case only for certain individuals such as those displaying hyperarousal symptoms. A major function of the central NE system is in promoting arousal and clinical and preclinical work has linked hyperarousal to excess NE activity. Thus NM-MRI could guide treatment decisions, consistent with future clinical practices where treatments are selected based on objective neurobiological measures rather than subjective clinical measures that are removed from the neurobiology underlying pathology.

We propose to recruit 60 individuals with trauma history (largely from an Operational Stress Injury Clinic), 30 of whom will meet CAPS-5 criteria for PTSD. This trauma-exposed group will be a representative sample spanning the full PTSD phenotype and thereby promoting an RDoC-inspired investigation of the neurobiological correlates of specific symptoms. All participants will undergo an MRI scan and clinical assessment. The MRI session will consist of an NM-MRI scan and a functional MRI scan during a fear conditioning procedure. LC NM-MRI signal will be measured via an automated LC segmentation pipeline.

For functional MRI scans, fear-related activation of the LC will be measured in native-space using the segmented LC as a localizer. Analyses will test if, as seen in our pilot data, clinical and physiological measures of arousal correlate to NM signal in the LC and to LC activation during fear conditioning.

Exploratory analyses will investigate the relationship of the LC NM signal to activation of structures within the fear circuit such as amygdala and prefrontal cortex. This study will provide the foundation to subsequently test pharmacological and non-pharmacological interventions that address hyperarousal symptoms and their neurobiological correlates. It will thus provide a novel and practical disease and treatment biomarker that we propose to introduce into the clinical care in PTSD.

### Background

PTSD is a burdensome and prevalent mental health issue for Armed Forces veterans. Amongst Regular Force Veterans having seen operational deployment between 1998-2015, 16.4% reported having PTSD. Hyperarousal is one of the syndromes underlying post-traumatic stress disorder (PTSD). This symptom cluster is characterized by hypervigilance, exaggerated startle, irritable or reckless behaviour, and sleep disturbance. Hyperarousal is common in PTSD and can be very harmful, leading to disability, physical health problems, and suicide. Earlier, more effective treatment of military PTSD will help to mitigate these deleterious downstream effects. While subtyping of PTSD currently relies on clinical assessment, a burgeoning understanding of neurobiological mechanisms underlying the etiology of PTSD opens the door to a future where biological measurements will be the preferred method to discriminate distinct pathologies within PTSD. While peripheral physiological assessments of hyperarousal currently exist, this symptom cluster may depend on dysregulation within the central nervous system and biomarkers of hyperarousal that track it here at the source may be best but are more challenging to find.

Furthermore, to be useful for treatment, biomarkers must be practical to implement broadly in clinical settings and must help indicate optimal treatment strategies.

In the current proposal we will test the utility of a novel putative biomarker, neuromelanin-sensitive MRI (NM-MRI), that is practical, reliable, and could link to pharmacological treatment strategies. NM-MRI is an imaging method that provides a practical and specific assay of the central norepinephrine (NE) system that could identify a subset of PTSD patients with NE imbalance and thereby allow targeted treatment attacking this imbalance using existing or experimental drugs (targeting the NE system) or using specific psychotherapy strategies.

The NE system of the brain is recognized as a key site of dysregulation in PTSD related to its role in stress response, arousal, and consolidation of fearful memories. A very recent and influential study has shown compelling evidence that hyperarousal in individuals with PTSD is linked to activity of the locus coeruleus (LC), the location of NE neurons in the brain. This confirms a decades-old theory linking the NE system to hyperarousal based on preclinical work and human PET imaging and genetics studies. The NE system is also an important target of common PTSD treatments including NE reuptake inhibitors (SNRIs and SNDRIs) and the α1-adrenoceptor antagonist drug prazosin which can effectively treat hyperarousal in some individuals. Furthermore, drugs in development for PTSD have action at NE receptors: a currently active clinical trial of brexpiprazole is testing target engagement of LC NE neurons (as indexed by pupillary diameter), iloperidone is a drug of interest due to high affinity for NE receptors, and recent findings show promise for treatment with propranolol prior to traumatic memory reactivation. Thus, a specific biomarker of NE imbalance in PTSD would be a highly useful tool in characterizing the illness, guiding treatment, and assessing efficacy of these new experimental treatments for targeted patients. A novel tool such as this may be provided by neuromelanin-sensitive MRI (NM-MRI; see FIG. 3).

Neuromelanin (NM) is a dark pigment that is formed from the breakdown of the catecholamine neurotransmitters NE and dopamine and is only present in the brain in catecholamine neurons (NE neurons in the LC and dopamine neurons in the substantia nigra). This pigment has unique properties that make it one of the only neurochemicals that can be quantified at high spatial resolution using MR imaging thereby allowing interrogation of the function of the NE system without the invasiveness and cost of PET imaging. It has the similar advantage of assaying brain chemistry to inform pharmacotherapy, but it is more practical for large-scale applications since it is inexpensive, brief (<10 minutes), non-invasive, and obtainable on any 3T MRI scanner. Because NM accumulates gradually over the lifespan and does not break down, NM-MRI has the added advantage of being a very stable measure, with high test-retest reliability. In the LC, this signal may provide a surrogate measure of sustained NE imbalance. This beneficial property ensures the signal will not change based on transient fluctuations in mental state or symptom severity (unlike some putative biomarkers).

We have gained extensive expertise in the acquisition and analysis of this imaging method. Our validation and development work has confirmed that NM-MRI is indeed sensitive to NM and is a marker of catecholamine neuron function and correlates to hyperarousal symptoms in PTSD, consistent with correlation of the signal to measures of anxiety and autonomic function in other populations. Despite this evidence in favor of its relevance to PTSD, no PTSD NM-MRI studies have yet been published.

While the stability over time of the NM-MRI signal allows for its use as an assay of long-term NE system function, its utility might be enhanced when combined with information from state-dependent measures of NE system function that can assay recent NE function. One such measure is the activity of the LC measured with BOLD fMRI during a fear conditioning paradigm. This paradigm will provide complementary information to the LC NM-MRI signal because LC activity promotes fear conditioning and generalization and enhanced fear conditioning is one model of the pathophysiology of PTSD. A further complimentary measure here is pupillometry, Assessment of pupillary dilation is a sensitive measure of reflexive, autonomic responding mediated by neural activity generated within the locus coeruleus.

NM-MRI signal in the LC is a biomarker of NE imbalance in the central nervous system. We evaluated this in Canadian Armed Forces (CAF) veterans with PTSD given the link between hyperarousal symptoms in PTSD and NE function. This supports the effort to move from clinical subtyping towards neurobiological subtyping of PTSD to promote targeted treatment and to accelerate new treatment discovery by preselecting likely-responders to experimental treatments. NM-MRI signal in the LC correlates to clinical and physiological measures of NE function in individuals with trauma exposure.

Evaluating the utility of the NM-MRI signal in the LC as a biomarker of long-term NE function in individuals exposed to trauma (and a possible moderating role of sex in this relationship).

LC NM-MRI signal positively correlates to severity of CAPS-5 hyperarousal symptoms, to skin conductance response during fear conditioning, and to velocity of pupil dilation in both sexes.

Evaluating the utility of BOLD fMRI activation of the LC during fear conditioning as an alternative biomarker of in-the-moment NE function which may complement the NM-MRI signal in relating to hyperarousal. Exploratory Aim 1b: To assess the correlation of LC measures (NM-MRI and BOLD) to fear-related BOLD activation of brain structures in the classical fear circuit [40]. Exploratory Aim 1c: To compare LC NM-MRI signal from trauma-exposed individuals meeting CAPS-5 criteria for PTSD to trauma-exposed individuals not meeting PTSD criteria.

### Preliminary Data

### Validation of NM-MRI as a Measure of the Function of Catecholamine Systems

NM-MRI is in fact sensitive to neuromelanin: NM-MRI signal corresponded to regional tissue concentration of NM in human post-mortem midbrain (β=0.87, t114=5.05, p=10-6, mixed-effects model, 116 measurements,7 specimens). Our in vivo PET imaging study confirmed the link between NM-MRI and function of catecholamine neurotransmitters (partial rho=0.69, p=0.004, n=18; in this study the catecholamine probed was not NE but dopamine, which generates a dopamine-related NM-MRI signal in the substantia nigra as opposed to the NE-related signal in the LC). NE and its precursor, dopamine, feed into the same metabolic pathway leading to NM formation thus our validation findings from the substantia nigra NM- MRI signal of the dopamine system should translate well to the LC NM-MRI signal of the NE system.

### Correlation of NM-MRI in the Locus Coeruleus to Hyperarousal Symptoms in PTSD

We examined LC NM-MRI signal in our dataset of 24 individuals treated at the Operational Stress Injury (OSI) Clinic at the Royal Ottawa Mental Health Centre (19 of which met CAPS criteria for PTSD). The LC NM-MRI signal was measured using established methods: the percent signal change in the LC is calculated relative to a reference region that does not contain neuromelanin.

Psychopathology was measured in this sample using the CAPS-5. From this scale we examined the hyperarousal symptom cluster. Consistent with our hypothesis, LC NM-MRI signal was positively correlated to severity of the CAPS-5 hyperarousal symptom cluster (r=0.52, p=0.019, partial correlation controlling for age, sex, PTSD diagnosis, depression severity [BDI total score], see FIG. 1). Similar to previous reports, LC NM-MRI signal was negatively correlated to depression severity (r=−0.48, p=0.033, partial correlation controlling for age, sex, PTSD diagnosis, and hyperarousal severity).

### Power Calculation

According to our data, we expect an effect size for the relationship between LC NM-MRI signal and hyperarousal symptoms to be close to r=0.52. Even assuming a somewhat weaker effect (r=0.4), to be conservative, we will have 90% power to detect a significant correlation with a sample size of n=60. Therefore, we propose to recruit 60 participants with trauma history. Based on our preliminary data in trauma-exposed veterans from the OSI clinic (FIG. 1), and internal data from the clinic we expect to observe a wide range of severity of hyperarousal symptoms in this population which will support our analysis focusing on this symptom dimension, an approach conforming with the RDoC approach [9].

### Study Participants

Study participants are be male and female Canadian Armed Forces veterans with a history of operational deployment (a proxy of trauma exposure), aged between 18-55 years. Individuals above 55 years of age are not be recruited to minimize confounds due to incipient LC degeneration in some individuals with advanced age. Consistent with the aims of the RDoC initiative [9], our study examines a single group of trauma-exposed individuals representing the full spectrum of trauma related symptomology. Given the heterogeneity of PTSD, this approach maximizes our ability to identify neurobiological correlates by enrolling individuals who have similarly experienced trauma but vary in the symptom domain of interest (hyperarousal). We maximize variability in this measure of interest but minimize variability in other clinical measures (e.g. trauma exposure, comorbidities, lifestyle factors that are important to match in PTSD control groups). It will be necessary to recruit n=66 participants to meet our target of 60 participants with usable data. Individuals with comorbid psychiatric disorders will be eligible to participate. Exclusion criteria include: active suicidal intent, major unstable medical illness, treatment with stimulant medications (>1 month lifetime), pregnancy, neurological disorder, and presence of any contraindication for MRI scanning. There will be no exclusion due to substance use or medication history (aside from stimulants, which may influence NM-MRI signal). Inclusive criteria such as these are consistent with many PTSD studies seeking to capture a representative sample in light of the prevalence of substance use disorders in this population and the heterogeneity of pharmacological agents prescribed (see Response to Previous Reviews for further discussion of these issues)

Recruitment of operationally deployed veterans in the community will occur in parallel via classified ads and word of mouth (e.g. from participants recruited at OSI clinic). To facilitate secondary analysis comparing trauma-exposed individuals with and without PTSD, we will ensure to enroll n=20 (from the total sample of 60 trauma-exposed veterans) who do not meet CAPS-5 criteria for PTSD. This breakdown of participants perfectly matches the proportion of individuals at the OSI clinic with a diagnosis of PTSD (66% in 2017). To properly assay both sexes and support analysis of sex effects we will ensure at minimum 40% of the sample is female (our current neuroimaging data collected from this clinic includes 7/24 females, 29%).

### Clinical Measures

Following screening and consent, all study participants undergo a 3-4 hour testing session conducted at The Royal Ottawa Mental Health Centre consisting of an MRI scan, physiological measurement, and clinical interview. The following clinical measures will be collected in all participants by interview or self-report: Interview measures: Clinician Administered PTSD Scale (CAPS-5, our primary clinical measure of interest), Structured Clinical Interview for DSM 5 (SCID-5); self-report measures: PTSD Checklist-5 (PCL-5), Pittsburgh Sleep Quality Index, Life Events Checklist, Beck Depression Inventory (BDI-II), Beck Anxiety Inventory (BAI), Dissociative Experiences Scale, Difficulty in Emotion Regulation Scale, the Chemical Use Abuse and Dependence Scale (CUAD), and Columbia Suicidality Severity Rating Scale.

### Magnetic resonance imaging (MRI) and physiological measures

All subjects will undergo MRI scanning using the 3T MR-PET Siemens Biograph scanner at the Royal Ottawa Mental Health Centre. This will include structural scans (T1 and T2-weighted scans), an NM-MRI scan, and a BOLD functional MRI scan during fear conditioning. Total scanning time for each participant will be around 50 minutes. A 32-channel headcoil is used for all scans. The NM-MRI scan is a 2D-GRE scan with magnetization transfer contrast and the following parameters: TR=260 ms, TE=2.68 ms, flip angle=40°, in-plane resolution=0.39×0.39 mm, FoV=162×200, matrix=416×512, number of slices=10, slice thickness=3.0 mm, magnetization transfer frequency offset=1200 Hz, number of excitations=8, acquisition time=8.04 minutes. BOLD-functional MRI images are acquired at high temporal and anatomical resolution with the following sequence parameters: 66 slices; TR=864 ms; TE=34.8 ms; Flip angle=52°; Matrix=88×90; FoV=208×97.8 mm2; Voxel size=2.3 mm isotropic; multiband acceleration factor=6. Also a spin echo sequence and a BO field map are collected to assist with correction of distortions and field inhomogeneities in BOLD images.

BOLD imaging takes place during a fear condition paradigm consisting of 3 different aversive conditioning tasks, each lasting for 7 minutes. During each task, participants are presented with two computer generated neutral faces (created using FaceGen; www.facegen.com). Each task has different faces. Within each task, one face (conditioned stimulus, CS+) is followed by a mild electrical shock (unconditioned stimulus) on the shin in 33% of the trials. The other face (control stimulus, CS−) is never followed by the shock. Skin conductance response (SCR) is calculated using Ledalab in Matlab, with a method called Continuous Decomposition Analysis (CDA). CDA performs a decomposition of SCR data into continuous signals of phasic (peaks i.e. after a CS+ event) and tonic activity (“baseline”). In practice, the Phasic Max (Maximal peak after a single event) is averaged for each event type (CS+, CS−). The final value that is compared is the CS+/− contrast. Positive contrast values indicate successful conditioning to CS+.

Pupil response measures are acquired using a Neuroptics PLR-3000 handheld pupilometer, a validated instrument producing highly reproducible measures [48]. The pupillometer's soft cup is placed against the eye to minimize outside light. The subject holds the untested eye open, and fixates on a spot 10 feet away on the wall. The protocol for use of this device was adapted from another study in PTSD.

Measurement is completed after 5-6 seconds during which pupil diameter at rest and in response to light pulse stimulation is measured. This procedure is repeated in 3 conditions of ambient light (light, dim, and dark: 350, 5, and 0 lux respectively) with a 4 minute interval between to adjust the light level. The characteristics of the light pulse are as follows: positive pulse stimulus, pulse intensity=50 uW, background intensity=0 uW, measurement duration=6.02 s, pulse duration=0.30 s, pulse onset=0.70 s (for ‘light’ condition) or positive pulse stimulus, pulse intensity =10uW, background intensity=0 uW, measurement duration =12.03 s, pulse duration=0.17 s, pulse onset=2.04 s (for ‘dim’ and ‘dark’ condition). Blood pressure at rest will also be measured just prior to the time of clinical assessment.

### Statistical Analysis

NM-MRI signal in the LC is measured directly from NM-MRI images using a custom automated method (FIG. 3). Primary analyses are linear regressions to predict either CAPS hyperarousal score, phasic skin conductance response to fear-conditioned stimuli, or pupillary dilation velocity based on the LC NM-MRI signal and including as covariates age, sex, and BDI severity. A secondary linear regression analysis test a model predicting hyperarousal symptom severity based on LC NM-MRI signal and also including the physiological measures (skin conductance, blood pressure, and pupillary diameter) as covariates to determine whether the LC NM-MRI signal makes an independent contribution to predict symptom severity beyond that of the more convenient peripheral measures. Secondary analysis will compare veterans meeting CAPS-5 criteria for PTSD (n=40) to the trauma-exposed veterans without PTSD (n=20) using a linear regression analysis controlling for age, sex, and depression severity. Additional secondary analyses will consider sex effects.

Analysis of functional MRI data will leverage the NM-MRI images to provide a segmentation of the LC as a subject-specific LC localizer and thus allow examination of LC activity during fear- conditioning. This method provides improved estimation of BOLD fMRI activity in the LC compared standard fMRI approaches (FIG. 2.). A final linear regression analysis will include LC NM-MRI signal and LC BOLD activation (contrast of conditioned stimulus minus unconditioned stimulus) to determine if they are complementary measures of long- and short-term NE system tone and independently predict hyperarousal symptoms and physiological measures. We will also exploit this rich dataset to explore correlation of our LC measures to fear-related activation of brain structures in the classical fear circuit such as amygdala, hypothalamus, and prefrontal cortex (FIG. 2) to develop an integrative model of brain mechanisms linking NE dysfunction to clinical symptoms.

### Sex- and Gender-Based Analysis

There are important sex differences in fear systems and sex-specific risk factors for PTSD and limited research into autonomic dysfunction in women with PTSD [51]. Thus, we will examine whether sex is a moderating factors in the relationship between the LC NM-MRI signal and hyperarousal We will do this by including a sex*LC signal interaction term in linear regression models predicting hyperarousal measures. We will recruit at minimum 40% females in our sample to support this analysis. We will also perform our primary analysis exclusively in men and in women to ensure the effect size is similar in both groups, supporting its use in both sexes.

### Example 5. Validation of the Algorithms Across Indications

Different neurological and psychiatric diseases have been associated with neuromelanin changes in two main regions, the substantia nigra pars compacta (SNc) and the locus coeruleus (LC). Differentiating between different disorders with similar clinical presentations solely based on presenting symptoms is difficult because the symptoms often overlap between related conditions.

The present disclosure describes the combined use of two fully automated algorithms to measure neuromelanin (NM) concentrations and volumes in two different brain regions (the SNc and the LC) to improve the ability to differentiate between related disorders. In this disclosure, the voxel based analysis algorithm (previously invented and patented at Columbia University) is used to measure NM in the SNc. However, since the LC is much smaller, and may not be as well suited to voxel based analysis on a 3T MRI (the most commonly available scanners in the clinic) a new algorithm was invented at University of Ottawa to measure NM in the LC. This LC algorithm is termed the segmented based analysis algorithm. This disclosure describes the combination of the two algorithms together in a software package that can be used to aid in the diagnosis and differentiation of neuropsychiatric disorders that are difficult to differentiate between based on symptoms alone.

In this disclosure, the software the voxel based analysis algorithm is used to measure NM in the SNc and the segmented based analysis algorithm is used to measure NM changes in the LC. The software reports the NM levels and volumes in both brain regions to the physician. The combination of these two algorithms together may increase the ability to differentiate between related neurological disorders. Their inclusion in a fully automated software allows the potential for their widespread use in the clinic.

The unmet medical need addressed here is the ability to differentiate between related disorders such as Parkinson's disease, multiple system atrophy, and progressive supranuclear palsy as well as different dementias such as Alzheimer's disease and dementia with Lewy bodies. The increase in ability to differentiate between related disorders, should increase the utility of the software in the clinic and should ultimately drive a more widespread use of the NM software as a medical device than would otherwise be possible with either algorithm alone.

### Results and Supporting Data by Indication

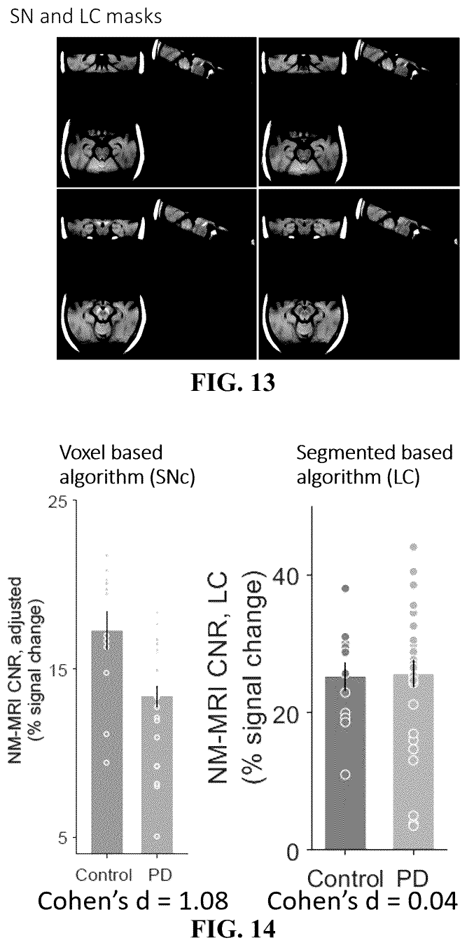

FIG. 13. The software automatically applies a mask to select the brain region for the SNc voxel based algorithm and a second mask to select the brain region for the LC segmented based algorithm.

### Parkinson's Disease

The two algorithms were validated by analyzing NM in the SNc and LC in patients with Parkinson's disease compared to healthy controls. The voxel based analysis algorithm found significant differences in NM contrast to noise ratio (CNR) in the SNc in patients with Parkinson's disease while the segmented based algorithm did not find any differences in the LC in this same patient population when compared to healthy controls (FIG. 14). This is consistent with the prior literature showing that the main changes in NM in Parkinson's disease are in the SNc, however this is the first time that this has been completed in a single brain scan utilizing dual fully automated algorithms.

### Alzheimer's Disease Diagnosis

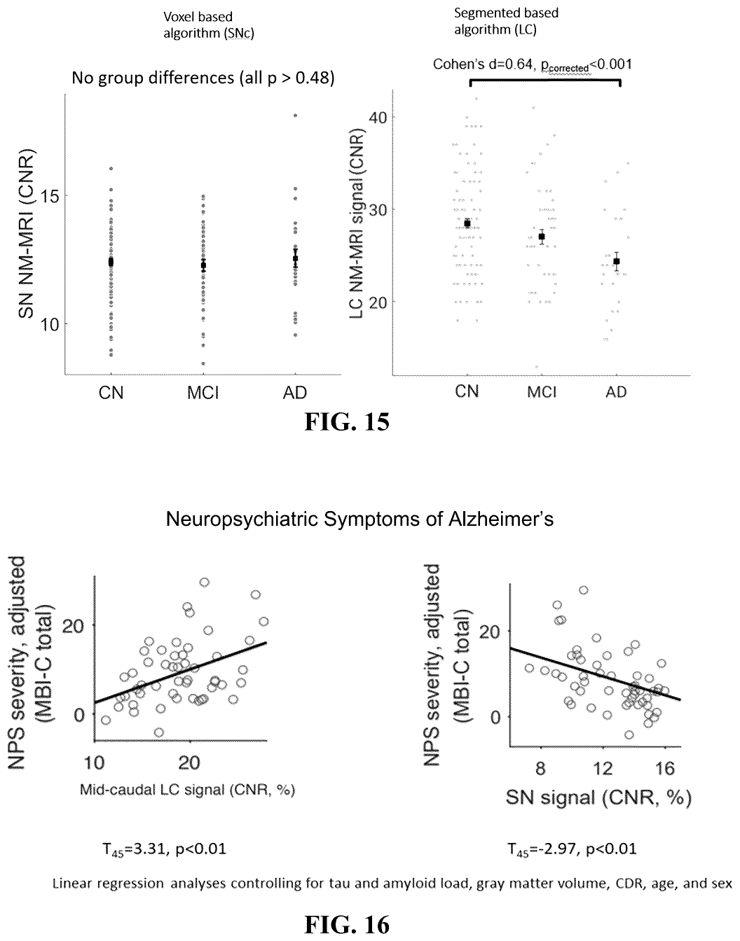

The algorithms were validated in patients with Alzheimer's disease compared to healthy controls. The segmented based analysis algorithm was applied analyze the LC while the voxel based algorithm was applied to analyze the SNc. In contrast to the patients with Parkinson's disease, the segmented based analysis algorithm found significant differences in NM in the LC in patients with Alzheimer's disease while the voxel based algorithm did not find any significant differences in the SNc in this same patient population when compared to healthy controls (FIG. 15). This is also consistent with the prior literature showing that the main NM changes in AD occur in the LC. The combination of these two datasets shows that when used together, the voxel based and segmented based algorithms are able to detect significant changes in NM in different brain regions simultaneously.

### Prediction of Neuropsychiatric Symptoms of Alzheimer's Disease

The combination of the two algorithms was used to help determine the presence of neuropsychiatric symptoms in patients with Alzheimer's disease. The segmented based analysis algorithm was applied analyze the LC while the volex based algorithm was applied to analyze the SNc (FIG. 16). The segmented analysis of the LC found that there are significant increases in NM in the LC compared to healthy controls. The voxel based algorithm shows there are significant decreases in NM in the SNc compared to healthy controls. This is the first time that NM levels in the SNc have been shown to significantly predict the presence of neuropsychiatric symptoms.

### Schizophrenia

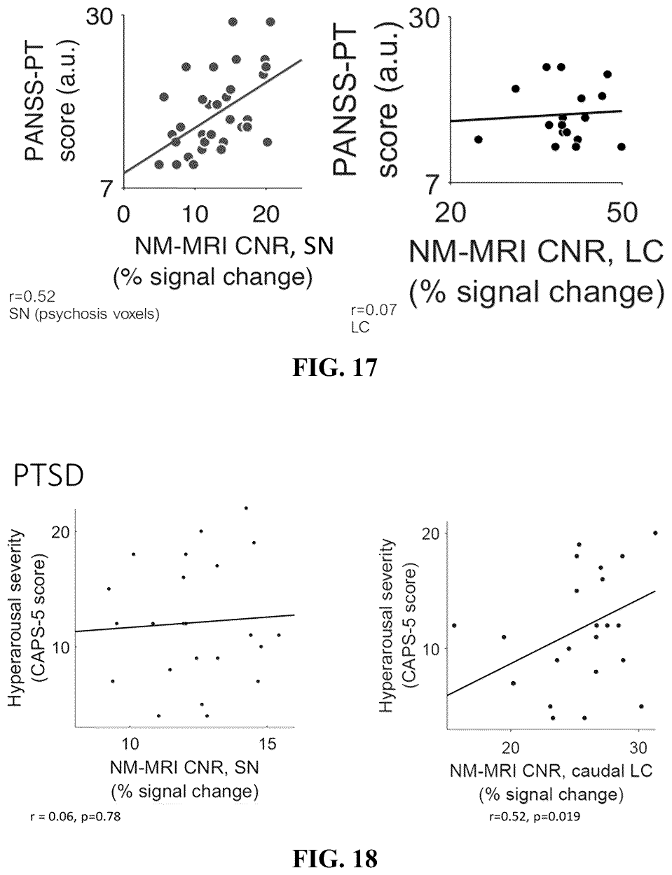

The algorithms were validated in patients with Schizophrenia. The segmented based analysis algorithm was applied analyze the LC while the voxel based algorithm was applied to analyze the SNc (FIG. 17). It was previously published that patients with schizophrenia have changes in NM levels in the SNc, however it is not known whether NM levels change in the LC. Compared to healthy controls, we found significant changes in NM levels in the SNc and that higher levels were associated with increasing psychosis severity as measured by the PANSS scale. Importantly, we did not find any significant change in NM levels in the LC. This is important because patients with Alzheimer's experience psychiatric symptoms that overlap with symptoms with schizophrenia (hallucinations and delusions). Importantly, this is the first data suggesting that NM levels measured in two brain regions may aid in the diagnosis of these illnesses. Importantly, psychotic symptoms in schizophrenia were associated with an increase in NM in the SNc only while the neuropsychiatric symptoms of Alzheimer's were associated with a decrease in NM in the SNc and an increase in the LC.

### PTSD

The combination of the algorithms were validated in patients with post traumatic stress disorder (PTSD).

The voxel based algorithm shows there are no significant association of disease severity with NM levels in the SNc compared to healthy controls (FIG. 18). The segmented based algorithm shows there are significant changes in the LC compared to healthy controls and that the increase NM levels are significantly associated with disease severity (right panel).

### Major Depressive Disorder

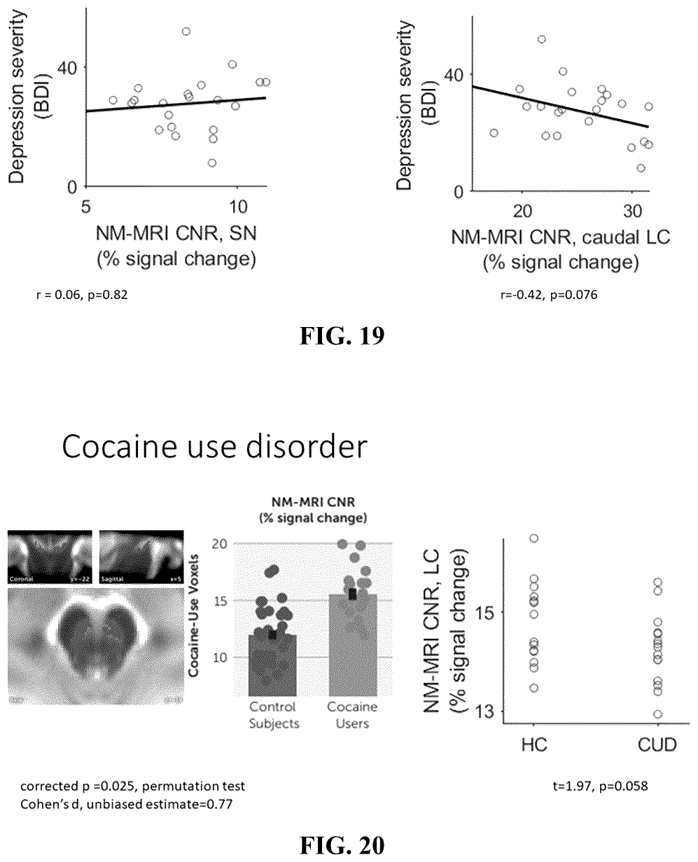

The algorithms were validated in patients with major depressive disorder compared to healthy controls. The segmented based analysis algorithm was applied analyze the LC while the voxel based algorithm was applied to analyze the SNc (FIG. 19). The voxel based algorithm shows there is a no significant difference in NM levels in the SNc compared to healthy controls (left panel). The segmented based algorithm shows there is a trend toward decreasing NM levels with increasing disease severity in the LC compared to healthy controls (right panel).

### Cocaine Use Disorder

The algorithms were validated in patients with cocaine use disorder compared to healthy controls. The segmented based analysis algorithm was applied analyze the LC while the voxel based algorithm was applied to analyze the SNc (FIG. 20). Application of the voxel based and segmented based algorithms to cocaine use disorder. The voxel based algorithm shows that increased NM in the SNc is significantly associated with cocaine use disorder compared to healthy controls (left panel). The segmented based algorithm shows there is a trend toward decreasing NM in the LC compared to healthy controls (right panel).

In one embodiment, the following summary table indicates various NM levels in the SN and LC and how this may guide the diagnosis of a specific disease in the patient or symptom severity.

| | SNc NM level | |
|---|---|---|
| | determined with | LC NM level determined |
| | the voxel based | with the segmented based |
| Indication | algorithm | algorithm |
| Schizophrenia with | The higher the level | Normal/Unchanged |
| psychotic symptoms | the more severe the | |
| (prognostic) | symptoms | |
| Cocaine use disorder | High, 14% change | Trend to low |
| (diagnostic) | Range 10-20% | |
| Parkinson's disease | Low (23% | Normal/Unchanged |
| (diagnostic) | reduction) change | |
| | (reduction) above | |
| | 10% | |
| Alzheimer's disease | Normal/Unchanged | Low less than (Range |
| without | | 10%-15% lower) |
| neuropsychiatric | | |
| symptoms | | |
| (diagnostic) | | |
| Alzheimer's disease | The lower the level | The higher the level the |
| neuropsychiatric | the more severe the | more severe the |
| symptoms | symptoms | symptoms |
| controlling for the | | |
| severity of the | | |
| illness (prognostic) | | |
| Major depressive | Normal/Unchanged | The lower the level the |
| disorder (prognostic) | | more severe the |
| | | symptoms |
| PTSD (diagnostic | Normal/Unchanged | High (19% higher) |
| and prognostic) | | Range greater than (10- |
| | | 25% increase) |
| | | Also the higher the level |
| | | the more severe the |
| | | symptoms |

### Example 6. Clinical Trial Validation Across Disease Indications

One of the difficulties facing the development of new therapeutics is the need to enrich enrollment for the patients who are most likely to benefit from the treatment and exclude patients who are not likely to respond. Accidental enrollment of the wrong patients is a contributing factor in clinical trial failures of new therapeutics and may result in developmental delays for otherwise effective treatments. This is especially necessary in PD as there is well-known clinical difficulty in differentiating Parkinson's disease from disorders in which Parkinsonism can be a presenting symptom including multiple system atrophy parkinsonian type (MSA-P) and progressive supranuclear palsy (PSP) or look-alikes such as some cases of essential tremor (ET) and idiopathic normal pressure hydrocephalus (iNPH). A retrospective analysis has shown that diagnostic accuracy of general neurologists for PD is 75% and for atypical parkinsonism including PSP and MSA the accuracy is only 61% for general neurologists and 71% for movement disorder specialists. The authors concluded that the high misdiagnosis rate increases the noise in clinical trials for PD. One study on ET showed that 25% of patients who were initially diagnosed with PD were later found to have ET. Similarly, a review of iNPH has reported that when gait dysfunction is present, iNPH may be difficult to differentiate from PD.

Imaging modalities that have shown promise in the differential diagnosis of PD have a number of limitations that reduce their utility as biomarkers in therapeutic clinical trials. These include positron emission tomography of tau protein (Tau-PET) which may aid in the diagnosis of PD vs PSP and the DaTscan which may aid in the differential diagnosis of PD vs ET and PD vs iNPH [16]. Both of these methods are expensive, require IV placement, expose the patient to a radioactive radiotracer, require lengthy prep and scan times, and require access to a PET scanner and SPECT scanner respectively. Altogether, these limitations make widespread deployment into large clinical trials unfeasible.

This study assesses NM-MRI as a biomarker to aid in the differential diagnosis of PD from patients with PSP, MSA, ET, and iNPH. Fifty subjects total (10 per group) with established diagnoses of PD (prior to treatment), PSP, MSA-P, ET, and iNPH (prior to shunting intervention), and 10 healthy controls will undergo NM-MRI scan in addition to clinical assessments including medical history, neurological examination including the Movement Disorder Society's Unified Parkinson's Disease Rating Scale (MDS-UPDRS). The absolute concentration and volume of neuromelanin in the SNc and LC of each brain hemisphere will be determined by Terrans NM-SAMD. Additionally, Terrans unique voxel- based analysis will be applied to determine voxel-based patterns for each disorder. The primary outcome will be the differences in absolute NM concentration and volume in the SNc and LC. The secondary outcome will be the regional specific voxel-based pattern of NM in the SNc and LC unique for each disorder. Recruitment will occur over a 12-month period. Specifically, PD, MSA, PSP, and ET patients occurs recruitment of patients with iNPH occurs prior to shunting intervention.

The study validates the biomarker NM to differentiate between Parkinson's spectrum disorders. NM-MRI could improve both the design and execution of future clinical trials by aiding in the differentiation of PD, PSP, MSA, ET, and iNPH. This is an inexpensive, fast, easily available, non-invasive biomarker with broad application for clinical trials by increasing the chance of clinical trials success for future therapeutics targeting PD or related disorders by reducing enrollment of patients with an incorrect diagnosis who would otherwise dilute the effects of a potentially valuable therapeutic. Finally, an enriched patient population may reduce the cost of future clinical trials by reducing the number of patients needed to reach statistical significance.

### REFERENCES

### Alzheimer's Disease References

Betts, M. J., Kirilina, E., Otaduy, M. C. G., Ivanov, D., Acosta-Cabronero, J., Callaghan, M. F., Lambert, C., Cardenas-Blanco, A., Pine, K., Passamonti, L., et al. (2019). Locus coeruleus imaging as a biomarker for noradrenergic dysfunction in neurodegenerative diseases. Brain 142, 2558-2571.

Lyketsos, C. G., Carrillo, M. C., Ryan, J.M., Khachaturian, A. S., Trzepacz, P., Amatniek, J., Cedarbaum, J., Brashear, R., and Miller, D. S. (2011). Neuropsychiatric symptoms in Alzheimer's disease. Alzheimers Dement 7, 532-539.

Lyketsos, C. G., Lopez, O., Jones, B., Fitzpatrick, A. L., Breitner, J., and DeKosky, S. (2002). Prevalence of neuropsychiatric symptoms in dementia and mild cognitive impairment: results from the cardiovascular health study. JAMA 288, 1475-1483.

German, D. C., Walker, B.S., Manaye, K., Smith, W. K., Woodward, D. J., and North, A. J. (1988). The human locus coeruleus: computer reconstruction of cellular distribution. J Neurosci 8, 1776-1788.

Geda, Y. E., Roberts, R. O., Knopman, D. S., Petersen, R. C., Christianson, T. J., Pankratz, V. S., Smith, G. E., Boeve, B. F., Ivnik, R. J., Tangalos, E. G., et al. (2008). Prevalence of neuropsychiatric symptoms in mild cognitive impairment and normal cognitive aging: population-based study. Arch Gen Psychiatry 65, 1193-1198.

Hwang, T. J., Masterman, D. L., Ortiz, F., Fairbanks, L. A., and Cummings, J. L. (2004). Mild cognitive impairment is associated with characteristic neuropsychiatric symptoms. Alzheimer Dis Assoc Disord 18, 17-21.

Ehrenberg, A. J., Suemoto, C. K., Franca Resende, E. P., Petersen, C., Leite, R. E. P., Rodriguez, R D., Ferretti-Rebustini, R. E. L., You, M., Oh, J., Nitrini, R., et al. (2018). Neuropathologic Correlates of Psychiatric Symptoms in Alzheimer's Disease. J Alzheimers Dis 66, 115-126.

Herrmann, N., Lanctot, K. L., and Khan, L. R. (2004). The role of norepinephrine in the behavioral and psychological symptoms of dementia. J Neuropsychiatry Clin Neurosci 16, 261-276.

Krell-Roesch, J., Vassilaki, M., Mielke, M. M., Kremers, W.K., Lowe, V. J., Vemuri, P., Machulda, M .M., Christianson, T. J., Syrjanen, J. A., Stokin, G. B., et al. (2019). Cortical beta-amyloid burden, neuropsychiatric symptoms, and cognitive status: the Mayo Clinic Study of Aging. Transl Psychiatry 9, 123.

Lussier, F. Z., Pascoal, T. A., Chamoun, M., Therriault, J., Tissot, C., Savard, M., Kang, M. S., Mathotaarachchi, S., Benedet, A. L., Parsons, M., et al. (2020). Mild behavioral impairment is associated with beta-amyloid but not tau or neurodegeneration in cognitively intact elderly individuals. Alzheimers Dement 16, 192-199.

Gatchel, J. R., Donovan, N. J., Locascio, J. J., Schultz, A. P., Becker, J. A., Chhatwal, J., Papp, K. V., Amariglio, R. E., Rentz, D. M., Blacker, D., et al. (2017). Depressive Symptoms and Tau Accumulation in the Inferior Temporal Lobe and Entorhinal Cortex in Cognitively Normal Older Adults: A Pilot Study. J Alzheimers Dis 59, 975-985.

Van Dam, D., Vermeiren, Y., Dekker, A. D., Naude, P. J., and Deyn, P. P. (2016). Neuropsychiatric Disturbances in Alzheimer's Disease: What Have We Learned from Neuropathological Studies? Curr Alzheimer Res 13, 1145-1164.

Allegri, R. F., Sarasola, D., Serrano, C. M., Taragano, F. E., Arizaga, R. L., Butman, J., and Lon, L. (2006). Neuropsychiatric symptoms as a predictor of caregiver burden in Alzheimer's disease. Neuropsychiatr Dis Treat 2, 105-110.

Bliwise, D. L. (2004). Sleep disorders in Alzheimer's disease and other dementias. Clin Cornerstone 6 Suppl 1A, S16-28.

Seignourel, P. J., Kunik, M. E., Snow, L., Wilson, N., and Stanley, M. (2008). Anxiety in dementia: a critical review. Clin Psychol Rev 28, 1071-1082.

Nelson, J. C., Delucchi, K., and Schneider, L. S. (2008). Efficacy of second generation antidepressants in late-life depression: a meta-analysis of the evidence. Am J Geriatr Psychiatry 16, 558-567.

Schneider, L. S., Dagerman, K., and Insel, P. S. (2006). Efficacy and adverse effects of atypical antipsychotics for dementia: meta-analysis of randomized, placebo-controlled trials. Am J Geriatr Psychiatry 14, 191-210.

Weintraub, D., Rosenberg, P. B., Drye, L. T., Martin, B. K., Frangakis, C., Mintzer, J. E., Porsteinsson, A. P., Schneider, L. S., Rabins, P. V., Munro, C. A., et al. (2010). Sertraline for the treatment of depression in Alzheimer disease: week-24 outcomes. Am J Geriatr Psychiatry 18, 332-340.

Showraki, A., Murari, G., Ismail, Z., Bar - fat, J. J., Fornazzari, L., Munoz, D. G., Schweizer, T. A., and Fischer, C. E. (2019). Cerebrospinal Fluid Correlates of Neuropsychiatric Symptoms in Patients with Alzheimer's Disease/Mild Cognitive Impairment: A Systematic Review. J Alzheimers Dis 71, 477-501.

Jellinger, K. A., and Bancher, C. (1998). Neuropathology of Alzheimer's disease: a critical update. J Neural Transm Suppl 54, 77-95.

Braak, H., and Braak, E. (1991). Neuropathological stageing of Alzheimer-related changes. Acta Neuropathol 82, 239-259.

Koppel, J., Acker, C., Davies, P., Lopez, O. L., Jimenez, H., Azose, M., Greenwald, B. S., Murray, P. S., Kirkwood, C. M., Kofler, J., et al. (2014). Psychotic Alzheimer's disease is associated with gender-specific tau phosphorylation abnormalities. Neurobiol Aging 35, 2021-2028.

Jacobs, H. I. L., Riphagen, J. M., Ramakers, I., and Verhey, F. R. J. (2019). Alzheimer's disease pathology: pathways between central norepinephrine activity, memory, and neuropsychiatric symptoms. Mol Psychiatry.

Gannon, M., Che, P., Chen, Y., Jiao, K., Roberson, E. D., and Wang, Q. (2015). Noradrenergic dysfunction in Alzheimer's disease. Front Neurosci 9, 220.

Satoh, A., and Iijima, K. M. (2019). Roles of tau pathology in the locus coeruleus (LC) in age-associated pathophysiology and Alzheimer's disease pathogenesis: Potential strategies to protect the LC against aging. Brain Res 1702, 17-28.

Vermeiren, Y., Van Dam, D., Aerts, T., Engelborghs, S., and De Deyn, P. P. (2014). Brain region-specific monoaminergic correlates of neuropsychiatric symptoms in Alzheimer's disease. J Alzheimers Dis 41, 819-833.

Matthews, K. L., Chen, C. P., Esiri, M. M., Keene, J., Minger, S. L., and Francis, P. T. (2002). Noradrenergic changes, aggressive behavior, and cognition in patients with dementia. Biol Psychiatry 51, 407-416.

Herrmann, N., Lanctot, K. L., Eryavec, G., and Khan, L. R. (2004). Noradrenergic activity is associated with response to pindolol in aggressive Alzheimer's disease patients. J Psychopharmacol 18, 215-220.

Peskind, E. R., Tsuang, D. W., Bonner, L. T., Pascualy, M., Riekse, R.G., Snowden, M. B., Thomas, R., and Raskind, M. A. (2005). Propranolol for disruptive behaviors in nursing home residents with probable or possible Alzheimer disease: a placebo-controlled study. Alzheimer Dis Assoc Disord 19, 23-28.

Teri, L., Reifler, B. V., Veith, R. C., Barnes, R., White, E., McLean, P., and Raskind, M. (1991). Imipramine in the treatment of depressed Alzheimer's patients: impact on cognition. J Gerontol 46, P372-377.

Forstl, H., Burns, A., Luthert, P., Cairns, N., Lantos, P., and Levy, R. (1992). Clinical and neuropathological correlates of depression in Alzheimer's disease. Psychol Med 22, 877-884.

Zubenko, G. S., and Moossy, J. (1988). Major depression in primary dementia. Clinical and neuropathologic correlates. Arch Neurol 45, 1182-1186.

Vermeiren, Y., Van Dam, D., Aerts, T., Engelborghs, S., and De Deyn, P.P. (2014). Monoaminergic neurotransmitter alterations in postmortem brain regions of depressed and aggressive patients with Alzheimer's disease. Neurobiol Aging 35, 2691-2700.

Zubenko, G. S., Moossy, J., Martinez, A. J., Rao, G., Claassen, D., Rosen, J., and Kopp, U. (1991). Neuropathologic and neurochemical correlates of psychosis in primary dementia. Arch Neurol 48, 619-624.

Aguero, C., Dhaynaut, M., Normandin, M. D., Amaral, A. C., Guehl, N. J., Neelamegam, R., Marquie, M., Johnson, K. A., El Fakhri, G., Frosch, M. P., et al. (2019). Autoradiography validation of novel tau PET tracer [F-18]-MK-6240 on human postmortem brain tissue. Acta Neuropathol Commun 7, 37.

Rowe, C. C., Pejoska, S., Mulligan, R. S., Jones, G., Chan, J. G., Svensson, S., Cselenyi, Z., Masters, C. L., and Villemagne, V. L. (2013). Head-to-head comparison of 11C-PiB and 18F-AZD4694 (NAV4694) for beta-amyloid imaging in aging and dementia. J Nucl Med 54, 880-886.

Cassidy, C. M., Zucca, F. A., Girgis, R. R., Baker, S. C., Weinstein, J. J., Sharp, M. E., Bellei, C., Valmadre, A., Vanegas, N., Kegeles, L. S., et al. (2019). Neuromelanin-sensitive MRI as a noninvasive proxy measure of dopamine function in the human brain. Proc Natl Acad Sci U S A 116, 5108-5117.

Sulzer, D., Cassidy, C., Horga, G., Kang, U. J., Fahn, S., Casella, L., Pezzoli, G., Langley, J., Hu, X. P., Zucca, F. A., et al. (2018). Neuromelanin detection by magnetic resonance imaging (MRI) and its promise as a biomarker for Parkinson's disease. NPJ Parkinsons Dis 4, 11.

Priovoulos, N., Jacobs, H. I. L., Ivanov, D., Uludag, K., Verhey, F. R. J., and Poser, B. A. (2018). High-resolution in vivo imaging of human locus coeruleus by magnetization transfer MRI at 3T and 7T. Neuroimage 168, 427-436.

Olivieri, P., Lagarde, J., Lehericy, S., Valabregue, R., Michel, A., Mace, P., Caille, F., Gervais, P., Bottlaender, M., and Sarazin, M. (2019). Early alteration of the locus coeruleus in phenotypic variants of Alzheimer's disease. Ann Clin Transl Neurol 6, 1345-1351.

Dordevic, M., Muller-Fotti, A., Muller, P., Schmicker, M., Kaufmann, J., and Muller, N. G. (2017). Optimal Cut-Off Value for Locus Coeruleus-to-Pons Intensity Ratio as Clinical Biomarker for Alzheimer's Disease: A Pilot Study. J Alzheimers Dis Rep 1, 159-167.

Takahashi, J., Shibata, T., Sasaki, M., Kudo, M., Yanezawa, H., Obara, S., Kudo, K., Ito, K., Yamashita, F., and Terayama, Y. (2015). Detection of changes in the locus coeruleus in patients with mild cognitive impairment and Alzheimer's disease: high-resolution fast spin-echo Tl-weighted imaging. Geriatr Gerontol Int 15, 334-340.

Sasaki, M., Shibata, E., Ohtsuka, K., Endoh, J., Kudo, K., Narumi, S., and Sakai, A. (2010). Visual discrimination among patients with depression and schizophrenia and healthy individuals using semiquantitative color-coded fast spin-echo Tl-weighted magnetic resonance imaging. Neuroradiology 52, 83-89.

Garcia-Lorenzo, D., Longo-Dos Santos, C., Ewenczyk, C., Leu-Semenescu, S., Gallea, C., Quattrocchi, G., Pita Lobo, P., Poupon, C., Benali, H., Arnulf, I., et al. (2013). The coeruleus/subcoeruleus complex in rapid eye movement sleep behaviour disorders in Parkinson's disease. Brain 136, 2120-2129.

Mather, M., Joo Yoo, H., Clewett, D. V., Lee, T. H., Greening, S. G., Ponzio, A., Min, J., and Thayer, J. F. (2017). Higher locus coeruleus MRI contrast is associated with lower parasympathetic influence over heart rate variability. Neuroimage 150, 329-335.

Ismail, Z., Aguera-Ortiz, L., Brodaty, H., Cieslak, A., Cummings, J., Fischer, C. E., Gauthier, S., Geda, Y. E., Herrmann, N., Kanji, J., et al. (2017). The Mild Behavioral Impairment Checklist (MBI-C): A Rating Scale for Neuropsychiatric Symptoms in Pre-Dementia Populations. J Alzheimers Dis 56, 929-938.

Cummings, J. L., Mega, M., Gray, K., Rosenberg-Thompson, S., Carusi, D. A., and Gornbein, J. (1994). The Neuropsychiatric Inventory: comprehensive assessment of psychopathology in dementia. Neurology 44, 2308-2314.

Kelly, S. C., He, B., Perez, S.E., Ginsberg, S. D., Mufson, E. J., and Counts, S.E. (2017). Locus coeruleus cellular and molecular pathology during the progression of Alzheimer's disease. Acta Neuropathol Commun 5, 8.

Betts, M. J., Cardenas-Blanco, A., Kanowski, M., Jessen, F., and Duzel, E. (2017). In vivo MRI assessment of the human locus coeruleus along its rostrocaudal extent in young and older adults. Neuroimage 163, 150-159.

Liu, K. Y., Acosta-Cabronero, J., Cardenas-Blanco, A., Loane, C., Berry, A. J., Betts, M. J., Kievit, R. A., Henson, R. N., Duzel, E., Cam, C. A. N., et al. (2019). In vivo visualization of age-related differences in the locus coeruleus. Neurobiol Aging 74, 101-111.

Liebe, T., Kaufmann, J., Li, M., Skalej, M., Wagner, G., and Walter, M. (2020). In vivo anatomical mapping of human locus coeruleus functional connectivity at 3 T MRI. Hum Brain Mapp.

DuBois, J. M., Rousset, O. G., Rowley, J., Porras-Betancourt, M., Reader, A. J., Labbe, A., Massarweh, G., Soucy, J. P., Rosa-Neto, P., and Kobayashi, E. (2016). Characterization of age/sex and the regional distribution of mGluR5 availability in the healthy human brain measured by high-resolution [(11)C]ABP688 PET. Eur J Nucl Med Mol Imaging 43, 152-162.

Mathotaarachchi, S., Pascoal, T. A., Shin, M., Benedet, A. L., Kang, M. S., Beaudry, T., Fonov, V. S., Gauthier, S., Rosa-Neto, P., and Alzheimer's Disease Neuroimaging, I. (2017). Identifying incipient dementia individuals using machine learning and amyloid imaging. Neurobiol Aging 59, 80-90.

Mathotaarachchi, S., Wang, S., Shin, M., Pascoal, T. A., Benedet, A. L., Kang, M. S., Beaudry, T., Fonov, V. S., Gauthier, S., Labbe, A., et al. (2016). VoxelStats: A MATLAB Package for Multi-Modal Voxel-Wise Brain Image Analysis. Front Neuroinform 10, 20.

Grothe, M. J., Barthel, H., Sepulcre, J., Dyrba, M., Sabri, 0., Teipel, S. J., and Alzheimer's Disease Neuroimaging, I. (2017). In vivo staging of regional amyloid deposition. Neurology 89, 2031-2038.

Cassidy, C. M., Balsam, P. D., Weinstein, J. J., Rosengard, R. J., Slifstein, M., Daw, N. D., Abi-Dargham, A., and Horga, G. (2018). A Perceptual Inference Mechanism for Hallucinations Linked to Striatal Dopamine. Curr Biol 28, 503-514 e504.

Sundermann, E. E., Katz, M. J., and Lipton, R. B. (2017). Sex Differences in the Relationship between Depressive Symptoms and Risk of Amnestic Mild Cognitive Impairment. Am J Geriatr Psychiatry 25, 13-22.

Forlani, C., Morri, M., Ferrari, B., Dalmonte, E., Menchetti, M., De Ronchi, D., and Atti, A. R. (2014). Prevalence and gender differences in late-life depression: a population-based study. Am J Geriatr Psychiatry 22, 370-380.

Lavretsky, H., Kurbanyan, K., Ballmaier, M., Mintz, J., Toga, A., and Kumar, A. (2004). Sex differences in brain structure in geriatric depression. Am J Geriatr Psychiatry 12, 653-657.

Oikawa, N., Ogino, K., Masumoto, T., Yamaguchi, H., and Yanagisawa, K. (2010). Gender effect on the accumulation of hyperphosphorylated tau in the brain of locus-ceruleus-injured APP-transgenic mouse. Neurosci Lett 468, 243-247.

Bangasser, D. A., Wiersielis, K. R., and Khantsis, S. (2016). Sex differences in the locus coeruleus-norepinephrine system and its regulation by stress. Brain Res 1641, 177-188.

Braun, D., and Feinstein, D. L. (2019). The locus coeruleus neuroprotective drug vindebumol normalizes behavior in the 5×FAD transgenic mouse model of Alzheimer's disease. Brain Res 1702, 29-37.

Cassidy, C. M., Norman, R., Manchanda, R., Schmitz, N., and Malla, A. (2010). Testing definitions of symptom remission in first-episode psychosis for prediction of functional outcome at 2 years. Schizophr Bull 36, 1001-1008.

Cassidy, C. M., Van Snellenberg, J. X., Benavides, C., Slifstein, M., Wang, Z., Moore, H., Abi-Dargham, A., and Horga, G. (2016). Dynamic Connectivity between Brain Networks Supports Working Memory: Relationships to Dopamine Release and Schizophrenia. J Neurosci 36, 4377-4388.

Rowley, J., Fonov, V., Wu, O., Eskildsen, S. F., Schoemaker, D., Wu, L., Mohades, S., Shin, M., Sziklas, V., Cheewakriengkrai, L., et al. (2013). White matter abnormalities and structural hippocampal disconnections in amnestic mild cognitive impairment and Alzheimer's disease. PLoS One 8, e74776.

Wu, L., Rowley, J., Mohades, S., Leuzy, A., Dauar, M. T., Shin, M., Fonov, V., Jia, J., Gauthier, S., Rosa-Neto, P., et al. (2012). Dissociation between brain amyloid deposition and metabolism in early mild cognitive impairment. PLoS One 7, e47905.

Waehnert, M. D., Dinse, J., Schafer, A., Geyer, S., Bazin, P. L., Turner, R., and Tardif, C.L. (2016). A subject-specific framework for in vivo myeloarchitectonic analysis using high resolution quantitative MRI. Neuroimage 125, 94-107.

Gauthier, S., Leuzy, A., and Rosa-Neto, P. (2014). How can we improve transfer of outcomes from randomized clinical trials to clinical practice with disease-modifying drugs in Alzheimer's disease? Neurodegener Dis 13, 197-199.

Ismail, Z., Agtiera-Ortiz, L., Brodaty H., Cieslak, A., Cummings, J., Fischer, C E., Gauthier, S., Geda, Y E, Herrmann, N, Kanji, J., et al. (2017). The Mild Behavioral Impairment Checklist (MBI-C): a rating scale for neuropsychiatric symptoms in pre-dementia populations. Journal of Alzheimer's disease 56.3, 929-938.

Creese, B., Brooker, H., Ismail, Z., Wesnes, K. A., Hampshire, A., Khan, Z., Megalogeni, M., Corbett, A., Aarsland, D., Ballard, C. (2019). Mild behavioral impairment as a marker of cognitive decline in cognitively normal older adults. The American Journal of Geriatric Psychiatry 27.8, 823-834.

Maust, D. T., Myra Kim, H., Seyfried L. S., Chiang, C., Kavanagh, J., Schneider, L. S., Kales, H. C. (2015). Antipsychotics, other psychotropics, and the risk of death in patients with dementia: number needed to harm. JAMA psychiatry 72.5, 438-445.

Ismail, Z. Smith, E. E., Geda, Y., Sultzer, D., Brodaty, H., Smith, G., Agtiera-Ortiz, L., Sweet, R., Miller, D., Lyketsos, C. G., et al. (2016). Neuropsychiatric symptoms as early manifestations of emergent dementia: provisional diagnostic criteria for mild behavioral impairment. Alzheimer's & Dementia 12.2, 195-202.

Porsteinsson, A. P., Drye, L. T., Pollock, B. G., Devanand, D. P., Frangakis, C., Ismail, Z., Marano, C., Meinert, C. L., Mintzer, J. E., Munro, C. E., et al. (2014). Effect of citalopram on agitation in Alzheimer disease: the CitAD randomized clinical trial. JAMA 311.7, 682-691.

### PTSD and MDD References

Hendrickson R C, Raskind M A (2016): Noradrenergic dysregulation in the pathophysiology of PTSD. Exp Neurol 284: 181-195.

Hendrickson R C, Raskind M A, Millard S P, Sikkema C, Terry G E, Pagulayan K F, et al. (2018): Evidence for altered brain reactivity to norepinephrine in Veterans with a history of traumatic stress. Neurobiol Stress 8: 103-111.

Naegeli C, Zeffiro T, Piccirelli M, Jaillard A, Weilenmann A, Hassanpour K, et al. (2018): Locus Coeruleus Activity Mediates Hyperresponsiveness in Posttraumatic Stress Disorder. Biol Psychiatry 83: 254-262.

Kang H K, Bullman T A, Smolenski D J, Skopp N A, Gahm G A, Reger M A (2015): Suicide risk among 1.3 million veterans who were on active duty during the Iraq and Afghanistan wars. Ann Epidemiol.

Jakupcak M, Cook J, Imel Z, Fontana A, Rosenheck R, McFall M (2009): Posttraumatic stress disorder as a risk factor for suicidal ideation in Iraq and Afghanistan war veterans. J Trauma Stress.

Pompili M, Sher L, Serafini G, Forte A, Innamorati M, Dominici G, et al. (2013): Posttraumatic stress disorder and suicide risk among veterans: A literature review. Journal of Nervous and Mental Disease.

Michopoulos V, Norrholm S D, Jovanovic T (2015): Diagnostic Biomarkers for Posttraumatic Stress Disorder: Promising Horizons from Translational Neuroscience Research. Biological Psychiatry.

Foa E B, Gillihan S J, Bryant R A (2013): Challenges and successes in dissemination of evidence-based treatments for posttraumatic stress: Lessons learned from prolonged exposure therapy for PTSD. Psychological Science in the Public Interest, Supplement.

Naegeli C, Zeffiro T, Piccirelli M, Jaillard A, Weilenmann A, Hassanpour K, et al. (2018): Locus Coeruleus Activity Mediates Hyperresponsiveness in Posttraumatic Stress Disorder. Biol Psychiatry 83: 254-262.

Hendrickson R C, Raskind M A (2016): Noradrenergic dysregulation in the pathophysiology of PTSD. Experimental Neurology.

Berridge C, Waterhouse B (2003): The locus coeruleus-noradrenergic system: modulation of behavioral state and state-dependent cognitive processes. Brain Res Rev 42: 33-84.

Samuels E, Szabadi E (2008): Functional Neuroanatomy of the Noradrenergic Locus Coeruleus: Its Roles in the Regulation of Arousal and Autonomic Function Part I: Principles of Functional Organisation. Curr Neuropharmacol 6: 235-253.

van Stegeren A H (2008): The role of the noradrenergic system in emotional memory. Acta Psychol (Amst).

Tully K, Bolshakov V Y (2010): Emotional enhancement of memory: How norepinephrine enables synaptic plasticity. Molecular Brain.

Berridge C W, Schmeichel B E, Espana R A (2012): Noradrenergic modulation of wakefulness/arousal. Sleep Medicine Reviews.

Aston-Jones G, Gonzalez M, Doran S (2007): Role of the locus coeruleus-norepinephrine system in arousal and circadian regulation of the sleep—wake cycle. Brain Norepinephrine Neurobiol Ther 157-195.

Chandler D J, Jensen P, McCall J G, Pickering A E, Schwarz L A, Totah N K (2019): Redefining Noradrenergic Neuromodulation of Behavior: Impacts of a Modular Locus Coeruleus Architecture. J Neurosci 39: 8239-8249.

Southwick S M, Bremner J D, Rasmusson A, Morgan C A, Arnsten A, Charney D S (1999): Role of norepinephrine in the pathophysiology and treatment of posttraumatic stress disorder. Biological Psychiatry.

American Psychiatric Association (2013): Diagnostic and Statisitical Manual of Mental Disorders, 5th Edition (DSM-5).

Blechert J, Michael T, Grossman P, Lajtman M, Wilhelm F H (2007): Autonomic and respiratory characteristics of posttraumatic stress disorder and panic disorder. Psychosomatic Medicine.

Shiner B, Leonard C E, Gui J, Cornelius S L, Schnurr PP, Hoyt J E, et al. (2020): Comparing Medications for DSM-5 PTSD in Routine V A Practice. J Clin Psychiatry.

Pitman R K, Brunet A, Bolshakov V, Gamache K, Nader K (2012): Toward reconsolidation blockade as a novel treatment for PTSD. Eur J Psychotraumatol.

Gamache K, Pitman R K, Nader K (2012): Preclinical evaluation of reconsolidation blockade by clonidine as a potential novel treatment for posttraumatic stress disorder. Neuropsychopharmacology.

Brunet A, Orr S P, Tremblay J, Robertson K, Nader K, Pitman R K (2008): Effect of post-retrieval propranolol on psychophysiologic responding during subsequent script-driven traumatic imagery in post-traumatic stress disorder. J Psychiatr Res.

Raskind M A, Peterson K, Williams T, Hoff D J, Hart K, Holmes H, et al. (2013): A trial of prazosin for combat trauma PTSD with nightmares in active-duty soldiers returned from Iraq and Afghanistan. Am J Psychiatry 170: 1003-1010.

Khachatryan D, Groll D, Booij L, Sepehry A A, Schatz C G (2016): Prazosin for treating sleep disturbances in adults with posttraumatic stress disorder: A systematic review and meta-analysis of randomized controlled trials. Gen Hosp Psychiatry 39: 46-52.

Sasaki M, Shibata E, Tohyama K, Takahashi J, Otsuka K, Tsuchiya K, et al. (2006): Neuromelanin magnetic resonance imaging of locus ceruleus and substantia nigra in Parkinson's disease. Neuroreport 17: 1215-1218.

Sulzer D, Cassidy C, Horga G, Kang UJ, Fahn S, Casella L, et al. (2018): Neuromelanin detection by magnetic resonance imaging (MRI) and its promise as a biomarker for Parkinson's disease. npj Park Dis 4.

Mather M, Joo Yoo H, Clewett D V., Lee TH, Greening SG, Ponzio A, et al. (2017): Higher locus coeruleus MRI contrast is associated with lower parasympathetic influence over heart rate variability. Neuroimage 150: 329-335.

Jacobs H I L, Priovoulos N, Poser B A, Pagen L H G, Ivanov D, Verhey F R J, Uludag K (2020): Dynamic behavior of the locus coeruleus during arousal-related memory processing in a multi-modal 7T fMRI paradigm. Elife.

Morris L S, Tan A, Smith D A, Grehl M, Han-Huang K, Naidich T P, et al. (2020): Sub-millimeter variation in human locus coeruleus is associated with dimensional measures of psychopathology: An in vivo ultra-high field 7-Tesla MRI study. Neurolmage Clin.

Weathers F W, Bovin M J, Lee D J, Sloan D M, Schnurr P P, Kaloupek D G, et al. (2018): The clinician-administered ptsd scale for DSM-5 (CAPS-5): Development and initial psychometric evaluation in military veterans. Psychol Assess 30: 383-395.

Beck A T, Ward C H, Mendelson M, Mock J, Erbaugh J (1961): An Inventory for Measuring Depression. Arch Gen Psychiatry.

Bernstein D P, Fink L (1997): Childhood Trauma Questionnaire: A Retrospective Self-Report (CTQ). Pearson.

Weathers F., Blake D D, Schnurr P P, Kaloupek D G, Marx B P, Keane T M (2013): The Life Events Checklist for DSM-5 (LEC-5). Natl Cent PTSD.

Weathers F W, Litz B T, Keane T M, Palmieri PA, Marx B P, Schnurr P P (2013): The PTSD Checklist for DSM-5 (PCL-5). Natl Cent PTSD.

Wolf E J, Mitchell K S, Sadeh N, Hein C, Fuhrman I, Pietrzak R H, Miller M W (2017): The Dissociative Subtype of PTSD Scale: Initial Evaluation in a National Sample of Trauma-Exposed Veterans. Assessment.

Watson D, Clark L A, Tellegen A (1988): Development and Validation of Brief Measures of Positive and Negative Affect: The PANAS Scales. J Pers Soc Psychol.

Beck A T, Steer R A (1990): Manual for the Beck Anxiety Inventory. Behaviour Research and Therapy.

Buysse D J, Reynolds C F, Monk T H, Berman S R, Kupfer D J (1989): The Pittsburgh sleep quality index: A new instrument for psychiatric practice and research. Psychiatry Res.

Posner K, Brown G K, Stanley B, Brent DA, Yershova K V., Oquendo M A, et al. (2011): The Columbia-suicide severity rating scale: Initial validity and internal consistency findings from three multisite studies with adolescents and adults. Am J Psychiatry.

Cassidy C M, Carpenter K M, Konova A B, Cheung V, Grassetti A, Zecca L, et al. (2020): Evidence for Dopamine Abnormalities in the Substantia Nigra in Cocaine Addiction Revealed by Neuromelanin-Sensitive MRI. Am J Psychiatry.

Keren N I, Lozar C T, Harris K C, Morgan P S, Eckert M A (2009): In vivo mapping of the human locus coeruleus. Neuroimage.

Brett M, Christoff K, Cusack R, Lancaster J (2001): Using the talairach atlas with the MNI template. Neuroimage.

Raskind M A, Peskind E R, Hoff D J, Hart KL, Holmes H A, Warren D, et al. (2007): A Parallel Group Placebo Controlled Study of Prazosin for Trauma Nightmares and Sleep Disturbance in Combat Veterans with Post-Traumatic Stress Disorder. Biol Psychiatry.

Raskind M A, Peskind E R, Kanter E D, Petrie E C, Radant A, Thompson C E, et al. (2003): Reduction of nightmares and other PTSD symptoms in combat veterans by prazosin: A placebo-controlled study. Am J Psychiatry.

Detweiler M, Pagadala B, Candelario J, Boyle J, Detweiler J, Lutgens B (2016): Treatment of Post-Traumatic Stress Disorder Nightmares at a Veterans Affairs Medical Center. J Clin Med 5: 117.

Montgomery S A (1997): Reboxetine: Additional benefits to the depressed patient. Journal of Psychopharmacology.

Eyding D, Lelgemann M, Grouven U, Harter M, Kromp M, Kaiser T, et al. (2010): Reboxetine for acute treatment of major depression: Systematic review and meta-analysis of published and unpublished placebo and selective serotonin reuptake inhibitor controlled trials. BMJ (Online).

Heck E, MacQueen G (2012): Noradrenergic and specific serotonergic antidepressants. Antidepressants and Major Depressive Disorder.

Papakostas G I, Thase M E, Fava M, Nelson J C, Shelton R C (2007): Are Antidepressant Drugs That Combine Serotonergic and Noradrenergic Mechanisms of Action More Effective Than the Selective Serotonin Reuptake Inhibitors in Treating Major Depressive Disorder? A Meta-analysis of Studies of Newer Agents. Biol Psychiatry.

Klimek V, Stockmeier C, Overholser J, Meltzer HY, Kalka S, Dilley G, Ordway GA (1997): Reduced levels of norepinephrine transporters in the locus coeruleus in major depression. J Neurosci.

Stockmeier C A, Rajkowska G (2004): Cellular abnormalities in depression: Evidence from postmortem brain tissue. Dialogues Clin Neurosci 6: 185-197.

Cassidy C M, Zucca F A, Girgis R R, Baker S C, Weinstein J J, Sharp M E, et al. (2019): Neuromelanin-sensitive MRI as a noninvasive proxy measure of dopamine function in the human brain. Proc Natl Acad Sci U S A 116: 5108-5117.

### EQUIVALENTS

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

1. An in vivo method of determining the progression of Alzheimer's disease over time in a subject, said method comprising:
(i) obtaining a first Neuromelanin-Magnetic Resonance Imaging (NM-Mill) scan at a first time point;
(ii) after step (i), obtaining a second NM-MRI scan at a second time point;
(iii) comparing the first neuromelanin magnetic resonance image to said second neuromelanin magnetic resonance image thereby determining whether a change in the level, signal and/or concentration of neuromelanin occurred between said first time point and said second time point.

2. The method according to claim 1, wherein if the change in the level, signal and/or concentration of neuromelanin at the second time point is more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 20%, or more than about 25% less than the level, signal and/or concentration of neuromelanin at the first time point, Alzheimer's disease is progressing.

3-4. (canceled)

5. A method of diagnosing a patient with Alzheimer's disease, said method comprising:
(i) measuring a level of neuromelanin
(ii) comparing the level of neuromelanin to a standard control,
(iii) optionally providing a diagnosis of Alzheimer's disease if the measured level of neuromelanin is lower relative to the standard control.

6. The method according to claim 5, further comprising determining a first signal intensity from said first neuromelanin magnetic resonance image and determining a second signal intensity from said second neuromelanin magnetic resonance image, wherein said comparing the first magnetic resonance image to said second magnetic resonance image comprises comparing the first signal intensity to the second signal intensity.

7. The method of claim 5, wherein a standard control is a level of neuromelanin present at approximately the same levels in a population of subjects, or said standard control is approximately the average level of neuromelanin present in a population of subjects.

8. The method according to claim 5, wherein a neuromelanin gradient phantom is used to measure the level, signal and/or concentration of neuromelanin.

9. The method according to claim 5, wherein a neuromelanin phantom concentration gradient is scanned about once per patient, about once an hour, about once a day, about once a week, or about once a month.

10. The method of claim 5, wherein a neuromelanin phantom gradient is scanned daily.

11. The method according to claim 5, wherein a neuromelanin phantom gradient is scanned with each patient.

12. The method according to claim 1, wherein if the change in the level, signal and/or concentration of neuromelanin at the second time point is more than about 5% less or more than about 10% less than the level, signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Alzheimer's disease is provided.

13. The method according to claim 1, wherein if the change in the level, signal and/or concentration of neuromelanin at the second time point is more than about 35% less, more than about 40% less, more than about 45% less, or more than about 50% less signal and/or concentration of neuromelanin at the first time point, wherein the first time point and the second time point are about 1 year, about 2 years, about 3 years, about 4 years, about 5 years, about 6 years, about 7 years, about 8 years, about 9 years, or about 10 years apart, a diagnosis of Alzheimer's disease is provided.

14. The method according to claim 1, wherein the second time point is about 3 months, about 6 months, about 9 months, about 12 months, about 2 years, about 3 years, about 4 years, about 5 years, about 6 years, about 7 years, about 8 years, about 9 years, about 10 years, about 15 years, about 20 years, about 25 years, or about 30 years after the first time point.

15. (canceled)

16. A method of determining if a subject has or is at risk of developing Alzheimer's disease, the method comprising analyzing one or more Neuromelanin-Magnetic Resonance Imaging (NM-MRI) scans of the subject's brain region of interest, wherein the analyzing comprises:
receiving imaging information of the brain region of interest; and
determining a NM concentration in the brain region of interest using segmented analysis based on the imaging information;
wherein the determining if a subject has or is at risk of developing Alzheimer's disease comprises:
(1) if the one or more NM-MRI scans has a decreased NM signal compared to a one or more control scans without Alzheimer's disease then the subject has or is at risk of developing Alzheimer's disease; or
(2) if the one or more NM-MRI scans has a NM signal comparable to the signal of a one or more control scans without Alzheimer's disease then the subject does not have or is not at risk of developing Alzheimer's disease.

17-23. (canceled)

24. The method according to claim 16, wherein a patient is diagnosed with Alzheimer's disease without displaying symptoms.

25. (canceled)

26. The method according to claim 1, wherein the comparing includes a segmented analysis.

27. The method according to claim 26, wherein the segmented analysis comprises determining at least one topographical pattern within the brain region of interest.

28. The method according to claim 26, wherein the method further comprises a calculation using a first value that represents a volume of a neuromelanin segment associated with the first neuromelanin magnetic resonance image and a second value that represents a volume of a neuromelanin segment associated with the second neuromelanin magnetic resonance image.

29. The method according to claim 26, wherein the segmented analysis region of interest is the substantia nigra.

30. The method according to claim 26, wherein the segmented analysis region of interest the locus coeruleus.

31-33. (canceled)

34. The method according to claim 16, wherein the method is used with a second imaging method, wherein the second imaging method is selected from the group consisting of positron emission tomography (PET), structural MRI, comprises functional MM (fMRI), blood oxygen level dependent (BOLD) fRMI, iron sensitive MRI, quantitative susceptibility mapping (QSM), diffusion tensor imaging DTI, and single photon emission computed tomography (SPECT), DaTscan and DaTquant.

35-63. (canceled)
